(19) United States
(12) Patent Application Publication
(10) Pub. No.: US 2025/0234703 A1
(43) Pub. Date: Jul. 17, 2025

(54) Organic Compound And Organic Light-Emitting Element Comprising Same

(71) Applicant: P&H TECH Co., Ltd, Gyeonggi-do (KR)

(72) Inventors: Seo-Yong HYUN, Gyeonggi-do (KR); Gwan-hee PARK, Gyeonggi-do (KR); Do Yeol YOON, Seoul (KR)

(73) Assignee: P&H TECH Co., Ltd, Gyeonggi-do (KR)

(21) Appl. No.: 18/850,420

(22) PCT Filed: Mar. 21, 2023
(86) PCT No.: PCT/KR2023/003691

§ 371 Date: Sep. 24, 2024

(30) Foreign Application Priority Data
Mar. 24, 2022 (KR) 10-2022-0036398

(51) Int. Cl. H10K 50/858; C07C 15/14; C07C 22/08; C07C 25/18; C07D 203/04; C07D 205/08; C07D 207/267; C07D 207/27; C07D 211/76; C07D 213/16; C07D 213/26

(52) U.S. Cl. CPC H10K 50/858; C07C 15/14; C07C 22/08; C07C 25/18; C07D 203/04; C07D 205/08; C07D 207/267; C07D 207/27; C07D 211/76; C07D 213/16; C07D 213/26; C07C 2603/74

(57) ABSTRACT

The present invention relates to an organic compound that can enhance the light efficiency of light extracted to the outside of an organic light-emitting element due to having a low refractive index, and thus can be effectively utilized as a material for a light efficiency improving layer provided in the organic light-emitting element. The compound according to the present invention can be employed in the light efficiency improving layer to achieve a high-efficiency, long-lifespan organic light-emitting element having improved light-emitting efficiency, color purity, and lifespan characteristics, as well as low-voltage driving characteristics, and thus can be effectively used in various lighting and display elements.

### TECHNICAL FIELD

The present invention relates to a novel organic compound that is employed as a material for a light efficiency improving layer (capping layer) provided in an organic light-emitting device, and to an organic light-emitting device in which device light-emitting properties such as low voltage driving, light-emitting efficiency, color purity, and service life are remarkably improved by employing the same in the light efficiency improving layer.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

However, in order for such an organic light emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light emitting device has not yet been sufficiently made.

Thus, further improvements in terms of efficiency and life characteristics are required for good stability, high efficiency, long lifetime, and large size of organic light emitting devices. Particularly, there is a strong need to develop materials constituting each organic layer of organic light emitting devices.

In addition, recently, research aimed at improving the characteristics of organic light emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

The efficiency of the organic light-emitting device may be divided into internal light-emitting efficiency and external light-emitting efficiency, the internal light-emitting efficiency is related to the efficiency of exciton production and light conversion in various organic layers interposed between a first electrode and a second electrode, such as a hole transport layer, a light-emitting layer, and an electron transport layer, the external light-emitting efficiency is the efficiency at which light generated in the organic layer is extracted to the outside of the organic light-emitting device, and in order to increase such a light extraction efficiency, a light efficiency improving layer (capping layer) with an optimally adjusted refractive index is applied.

However, there is an urgent need for designing and developing an optimized light efficiency improving layer such that it is possible to implement a highly efficient device without impairing process efficiency such as deposition and other light-emitting characteristics including the service life characteristics and the like of a device.

### DISCLOSURE

### Technical Problem

Thus, the present invention has been made in an effort to provide a novel organic compound which may be employed in a light efficiency improving layer (capping layer) provided in an organic light-emitting device to implement excellent luminescent properties such as low-voltage driving of the device and improved luminous efficiency, and an organic light-emitting device including the same.

### Technical Solution

In order to solve the above problems, the present invention provides a novel organic compound for a light efficiency improving layer (capping layer) represented by the following [Formula I].

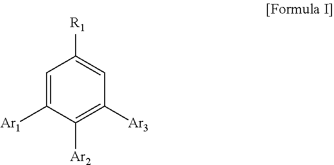

The characteristic structure of [Formula I] above and the definitions of the specific compound implemented by the same, R₁, and Ar₁ to Ar₃ will be described later. Further, the present invention provides an organic light-emitting device including a first electrode, a second electrode, and an organic layer having one or more layers disposed between the first electrode and the second electrode, in which the organic light-emitting device further includes a light efficiency improving layer (capping layer) formed on at least one side opposite to the organic layer at the top or bottom part of the first electrode and the second electrode and the light efficiency improving layer includes the organic compound represented by [Formula I].

### Advantageous Effects

The organic compound according to the present invention has a low refractive index and can improve the efficiency of light extracted to the outside of an organic light emitting device, and thus can be usefully used as a material for a light efficiency improving layer provided in the organic light emitting device. Accordingly, by employing the compound according to the present invention in the light efficiency improving layer, it is possible to implement a highly efficient and long service life organic light-emitting device having improved light-emitting efficiency, color purity, service life characteristics, as well as low voltage driving characteristics, and the like, so the organic light-emitting device can be usefully used in various lighting and display devices, and the like.

### BRIEF DESCRIPTION OF DRAWINGS

FIG. 1 is a schematic cross-sectional view illustrating the concept and configuration of an organic light-emitting device according to the present invention.

FIG. 2 is a schematic cross-sectional view illustrating the configuration of an organic light-emitting device according to an embodiment of the present invention.

FIG. 3 is a schematic cross-sectional view illustrating the configuration of an organic light-emitting device according to an embodiment of the present invention.

### BEST MODE FOR CARRYING OUT INVENTION

Hereinafter, the present invention will be described in more detail.

The present invention relates to an organic light emitting compound represented by Formula I below, which is employed as a material for a light efficiency improving layer provided in an organic light-emitting device to achieve low-voltage driving of the device and luminescent properties such as excellent luminous efficiency, color purity, etc.

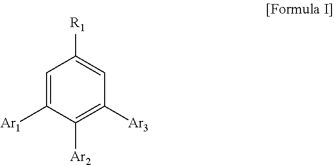

In Formula I above,
R₁ is selected from a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atom, and the following [Structural Formula 1].

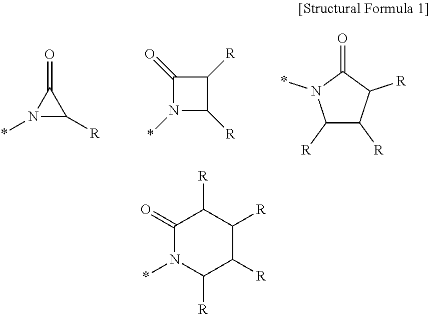

In Structural Formula 1 above,
R is each independently selected from hydrogen, deuterium, a cyano group, a halogen group, a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms.
Ar₁ to Ar₃ are the same as or different from each other, and each independently selected from a substituted or unsubstituted aryl group having 6 to 30 carbon atoms, a substituted or unsubstituted heteroaryl group having 2 to 30 carbon atoms, and [Structural Formula 1] above.

According to an embodiment of the present invention, the compound represented by [Formula I] above may be represented by the following [Formula I-1] to [Formula I-3].

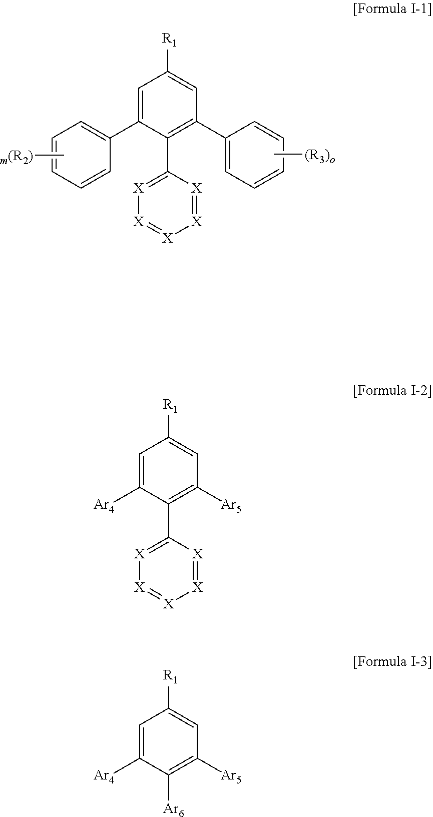

In [Formula I-1] to [Formula I-3] above,
R₁ is the same as the definition in [Formula I] above.
R₂ and R₃ are the same as or different from each other, and each independently selected from hydrogen, deuterium, a cyano group, a halogen group, a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atoms, and [Structural Formula 1] above.
m and o are each an integer from 0 to 5, and when m and o are 2 or higher, a plurality of R₂ and R³ are each the same as or different from each other.
X is each independently N or CR₄, R₄ is each independently selected from hydrogen, deuterium, a cyano group, a halogen group, a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atoms, and [Structural Formula 1] above, and the plurality of X and R⁴ are each the same as or different from each other.
Ar₄ to Ar₆ are the same as or different from each other, and each independently selected from the following [Structural Formula 1].

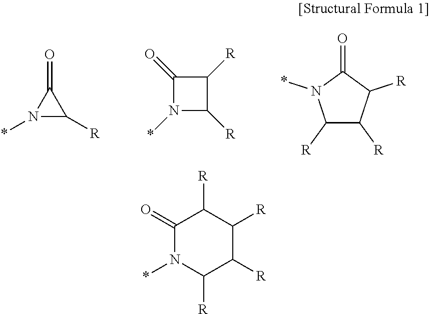

In Structural Formula 1 above,
R is each independently selected from hydrogen, deuterium, a cyano group, a halogen group, a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms.

Meanwhile, in the definitions of R, R₁ to R₃, and Ar₁ to Ar₆, ‘substituted or unsubstituted’ means that each of R, R₁, and Ar₁ to Ar₃ is substituted with one or two or more substituents selected from the group consisting of deuterium, a halogen group, a cyano group, a nitro group, a hydroxyl group, a silyl group, an amine group, a halogenated alkyl group, a deuterated alkyl group, a cycloalkyl group, a heterocycloalkyl group, an aryl group, a heteroaryl group, an alkylsilyl group, an arylsilyl group, and the following [Structural Formula 1], is substituted with a substituent to which two or more substituent among the above substituents are linked, or has no substituent.

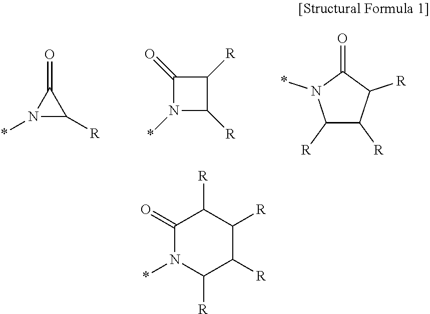

In Structural Formula 1 above,
R is each independently any one selected from hydrogen, deuterium, a cyano group, a halogen group, an alkyl group having 1 to 20 carbon atoms, a halogenated alkyl group having 1 to 20 carbon atoms, a cycloalkyl group having 3 to 20 carbon atoms, an aryl group having 6 to 30 carbon atoms, and a heteroaryl group having 2 to 30 carbon atoms.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkyl group may be substituted with deuterium, a halogen group, or the like to be a deuterated alkyl group or a halogenated alkyl group.

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the amine group may be —NH₂, an alkylamine group, an arylamine group, a heteroaryl amino group, an arylheteroarylamine group, etc., the aryl (heteroaryl)amine group means an amine substituted with an aryl group and/or heteroaryl group, and the alkylamine group means an amine substituted with an alkyl group. Examples of the aryl (heteroaryl)amine group include a substituted or unsubstituted mono aryl (heteroaryl)amine group, a substituted or unsubstituted diaryl (heteroaryl)amine group, or a substituted or unsubstituted triaryl (heteroaryl)amine group, wherein the aryl group and the heteroaryl group in the aryl (heteroaryl)amine group are the same as the definition of the aryl group and the heteroaryl group, and the alkyl group in the alkylamine group is also the same as the definition of the alkyl group.

For example, the arylamine group includes a phenylamine group, a naphthylamine group, a biphenylamine group, an anthracenylamine group, a 3-methyl-phenylamine group, a 4-methyl-naphthylamine group, and a 2-methyl-biphenyl amine group, a 9-methyl-anthracenylamine group, a diphenyl amine group, a phenylnaphthylamine group, a ditolylamine group, a phenyltolylamine group, a triphenylamine group, etc., but is not limited thereto.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (Cl), and bromine (Br).

The organic compound according to the present invention represented by Formula I may be used as a material for the light efficiency improving layer (capping layer) provided in the organic light emitting device due to its structural specificity.

Preferred specific examples of the organic compound represented by Formula I according to the present invention include the following compounds but are not limited thereto.

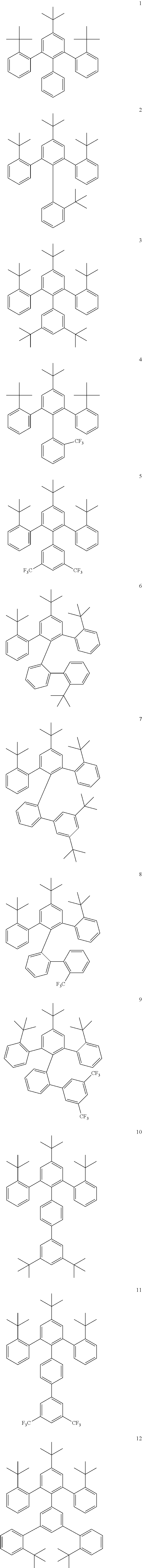

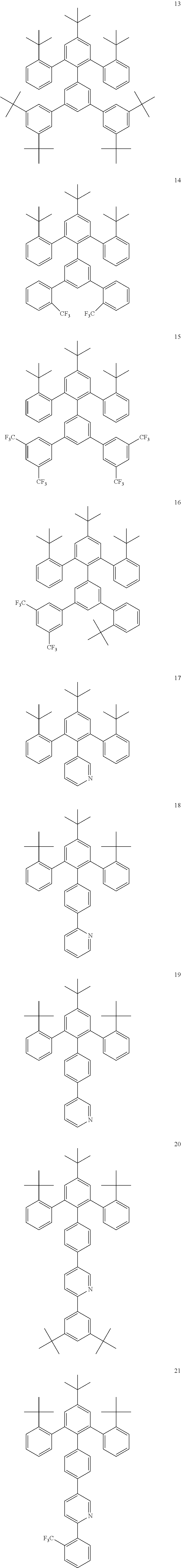

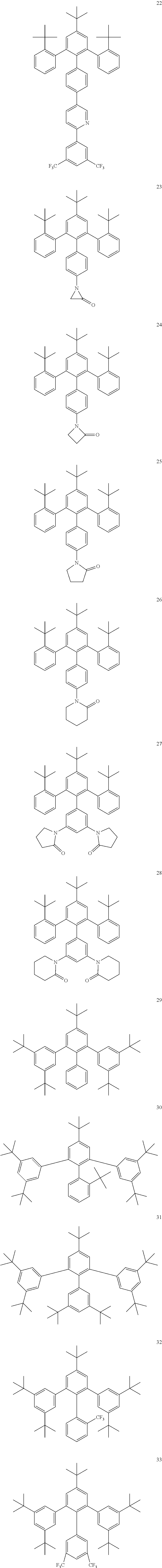

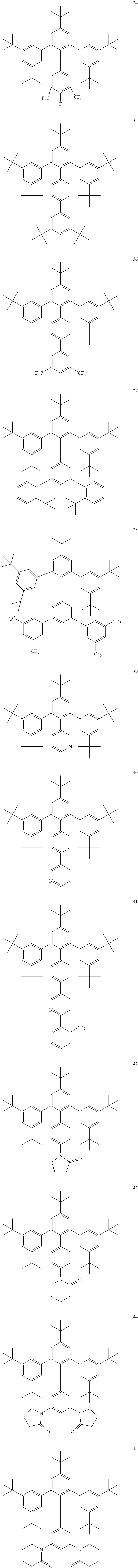

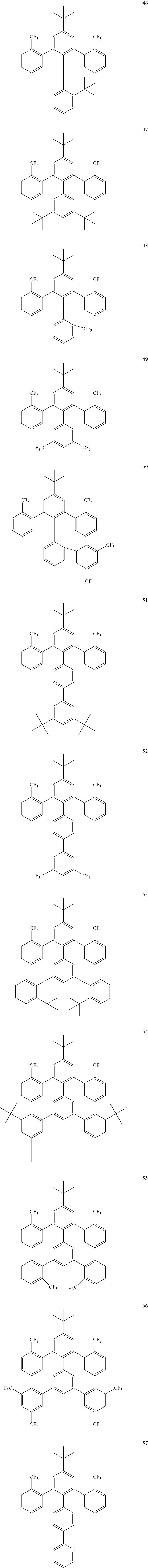

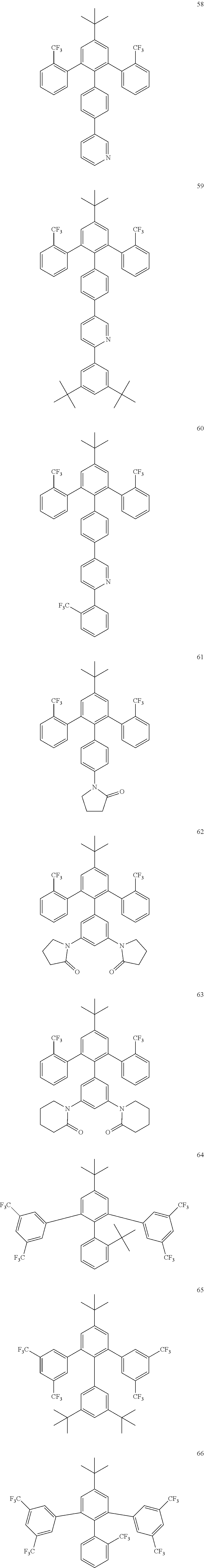

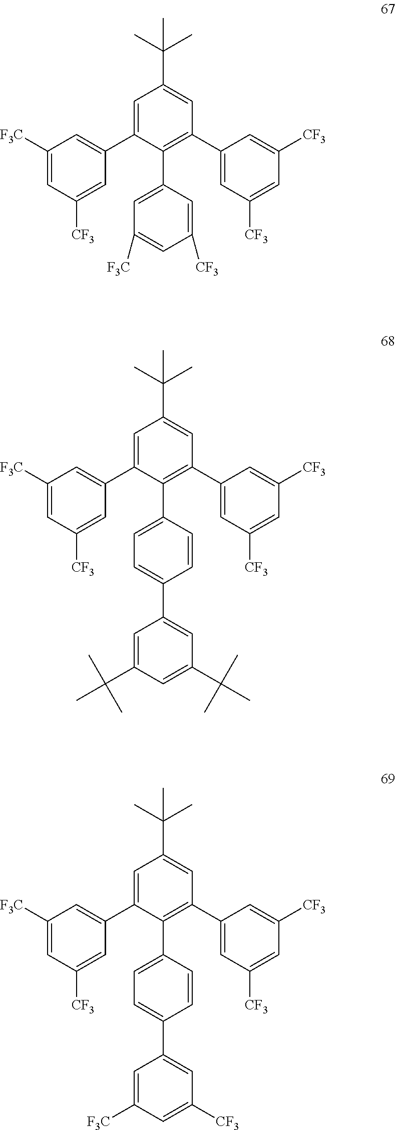 [SMILES: CC(C)(C)c1cc(-c2cc(C(F)(F)F)cc(C(F)(F)F)c2)c(-c2cc(C(F)(F)F)cc(C(F)(F)F)c2)c(-c2cc(C(F)(F)F)cc(C(F)(F)F)c2)c1 ; CC(C)(C)c1cc(-c2ccc(-c3c(-c4cc(C(F)(F)F)cc(C(F)(F)F)c4)cc(C(C)(C)C)cc3-c3cc(C(F)(F)F)cc(C(F)(F)F)c3)cc2)cc(C(C)(C)C)c1 ; CC(C)(C)c1cc(-c2cc(C(F)(F)F)cc(C(F)(F)F)c2)c(-c2ccc(-c3cc(C(F)(F)F)cc(C(F)(F)F)c3)cc2)c(-c2cc(C(F)(F)F)cc(C(F)(F)F)c2)c1]

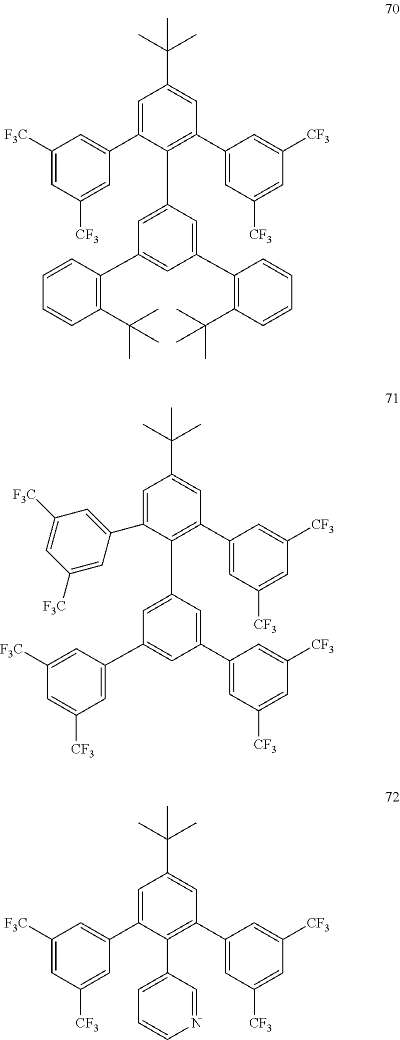 [SMILES: CC(C)(C)c1cc(-c2cc(C(F)(F)F)cc(C(F)(F)F)c2)c(-c2cc(-c3ccccc3C(C)(C)C)cc(-c3ccccc3C(C)(C)C)c2)c(-c2cc(C(F)(F)F)cc(C(F)(F)F)c2)c1 ; CC(C)(C)c1cc(-c2cc(C(F)(F)F)cc(C(F)(F)F)c2)c(-c2cc(-c3cc(C(F)(F)F)cc(C(F)(F)F)c3)cc(-c3cc(C(F)(F)F)cc(C(F)(F)F)c3)c2)c(-c2cc(C(F)(F)F)cc(C(F)(F)F)c2)c1 ; CC(C)(C)c1cc(-c2cc(C(F)(F)F)cc(C(F)(F)F)c2)c(-c2cccnc2)c(-c2cc(C(F)(F)F)cc(C(F)(F)F)c2)c1]

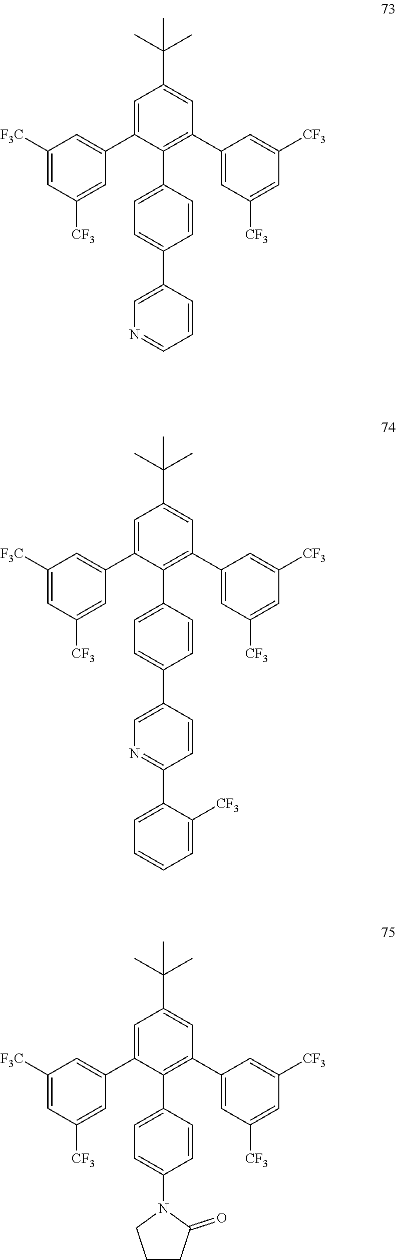 [SMILES: CC(C)(C)c1cc(-c2cc(C(F)(F)F)cc(C(F)(F)F)c2)c(-c2ccc(-c3cccnc3)cc2)c(-c2cc(C(F)(F)F)cc(C(F)(F)F)c2)c1 ; CC(C)(C)c1cc(-c2cc(C(F)(F)F)cc(C(F)(F)F)c2)c(-c2ccc(-c3ccc(-c4ccccc4C(F)(F)F)nc3)cc2)c(-c2cc(C(F)(F)F)cc(C(F)(F)F)c2)c1 ; CC(C)(C)c1cc(-c2cc(C(F)(F)F)cc(C(F)(F)F)c2)c(-c2ccc(N3CCCC3=O)cc2)c(-c2cc(C(F)(F)F)cc(C(F)(F)F)c2)c1]

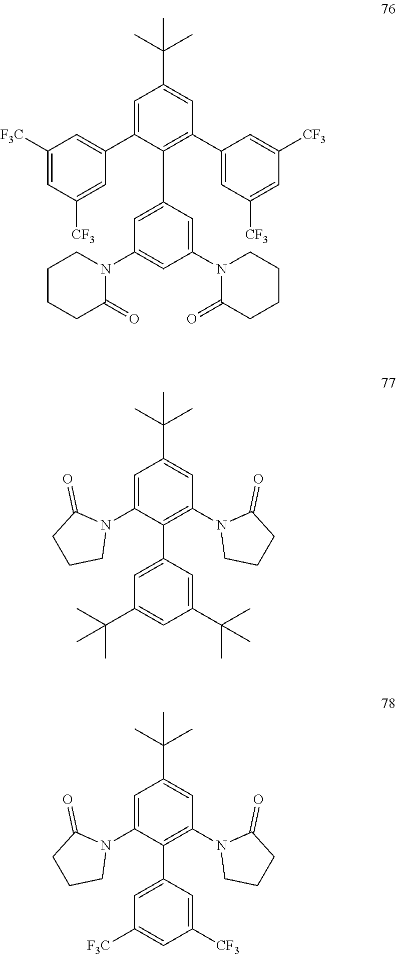 [SMILES: CC(C)(C)c1cc(-c2cc(C(F)(F)F)cc(C(F)(F)F)c2)c(-c2cc(N3CCCCC3=O)cc(N3CCCCC3=O)c2)c(-c2cc(C(F)(F)F)cc(C(F)(F)F)c2)c1 ; CC(C)(C)c1cc(-c2c(N3CCCC3=O)cc(C(C)(C)C)cc2N2CCCC2=O)cc(C(C)(C)C)c1 ; CC(C)(C)c1cc(N2CCCC2=O)c(-c2cc(C(F)(F)F)cc(C(F)(F)F)c2)c(N2CCCC2=O)c1]

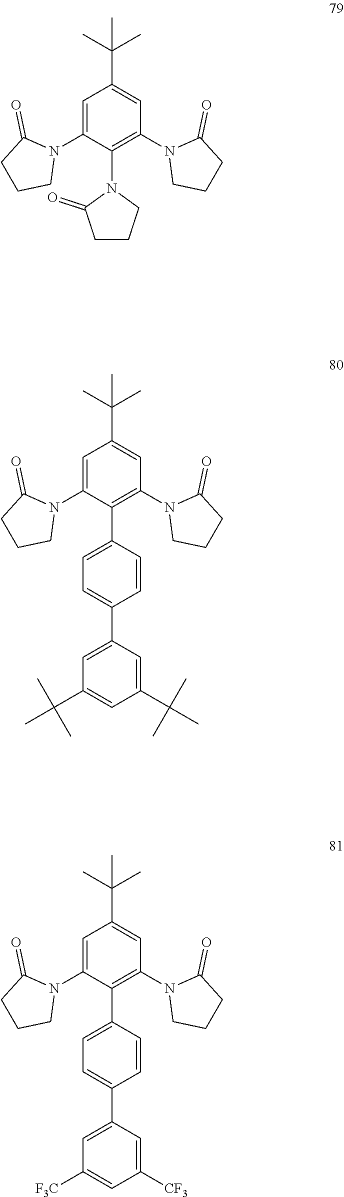 [SMILES: CC(C)(C)c1cc(N2CCCC2=O)c(N2CCCC2=O)c(N2CCCC2=O)c1 ; CC(C)(C)c1cc(-c2ccc(-c3c(N4CCCC4=O)cc(C(C)(C)C)cc3N3CCCC3=O)cc2)cc(C(C)(C)C)c1 ; CC(C)(C)c1cc(N2CCCC2=O)c(-c2ccc(-c3cc(C(F)(F)F)cc(C(F)(F)F)c3)cc2)c(N2CCCC2=O)c1]

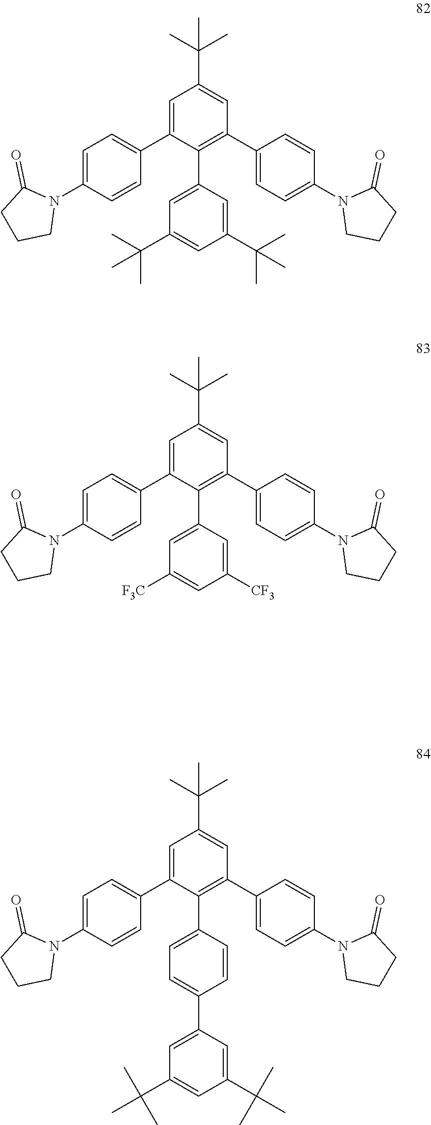 [SMILES: CC(C)(C)c1cc(-c2c(-c3ccc(N4CCCC4=O)cc3)cc(C(C)(C)C)cc2-c2ccc(N3CCCC3=O)cc2)cc(C(C)(C)C)c1 ; CC(C)(C)c1cc(-c2ccc(N3CCCC3=O)cc2)c(-c2cc(C(F)(F)F)cc(C(F)(F)F)c2)c(-c2ccc(N3CCCC3=O)cc2)c1 ; CC(C)(C)c1cc(-c2ccc(-c3c(-c4ccc(N5CCCC5=O)cc4)cc(C(C)(C)C)cc3-c3ccc(N4CCCC4=O)cc3)cc2)cc(C(C)(C)C)c1]

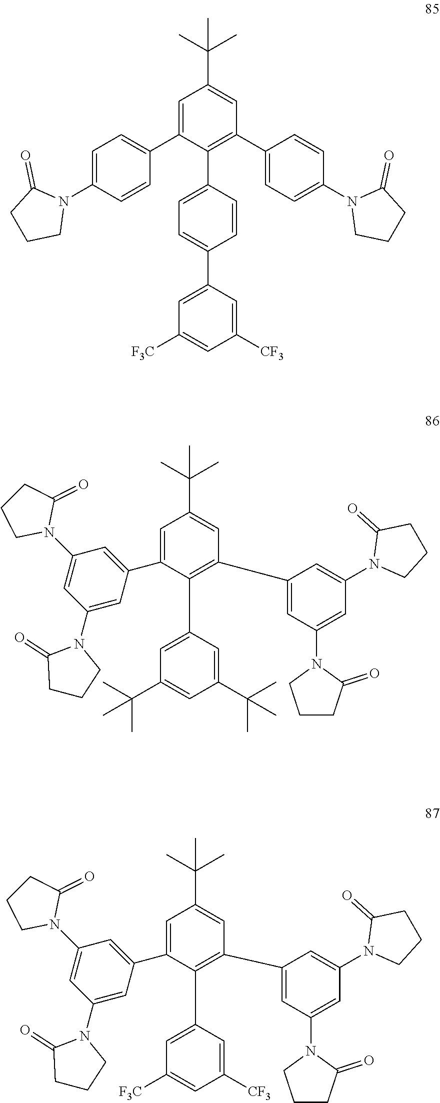 [SMILES: CC(C)(C)c1cc(-c2ccc(N3CCCC3=O)cc2)c(-c2ccc(-c3cc(C(F)(F)F)cc(C(F)(F)F)c3)cc2)c(-c2ccc(N3CCCC3=O)cc2)c1 ; CC(C)(C)c1cc(-c2c(-c3cc(N4CCCC4=O)cc(N4CCCC4=O)c3)cc(C(C)(C)C)cc2-c2cc(N3CCCC3=O)cc(N3CCCC3=O)c2)cc(C(C)(C)C)c1 ; CC(C)(C)c1cc(-c2cc(N3CCCC3=O)cc(N3CCCC3=O)c2)c(-c2cc(C(F)(F)F)cc(C(F)(F)F)c2)c(-c2cc(N3CCCC3=O)cc(N3CCCC3=O)c2)c1]

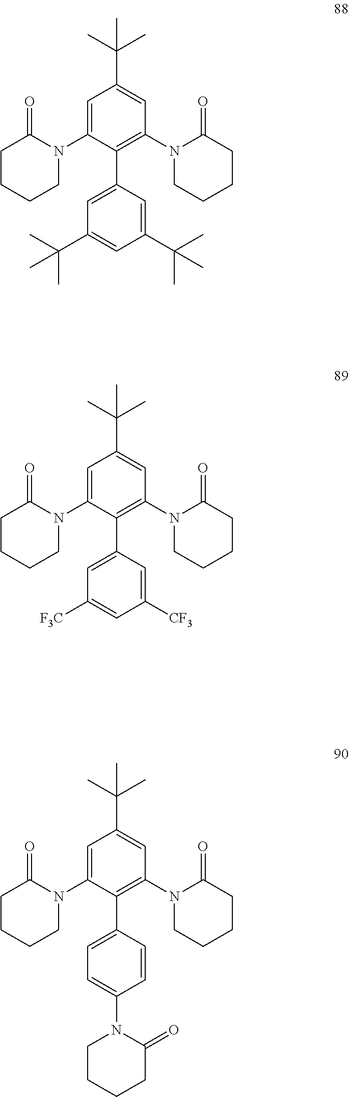 [SMILES: CC(C)(C)c1cc(-c2c(N3CCCCC3=O)cc(C(C)(C)C)cc2N2CCCCC2=O)cc(C(C)(C)C)c1 ; CC(C)(C)c1cc(N2CCCCC2=O)c(-c2cc(C(F)(F)F)cc(C(F)(F)F)c2)c(N2CCCCC2=O)c1 ; CC(C)(C)c1cc(N2CCCCC2=O)c(-c2ccc(N3CCCCC3=O)cc2)c(N2CCCCC2=O)c1]

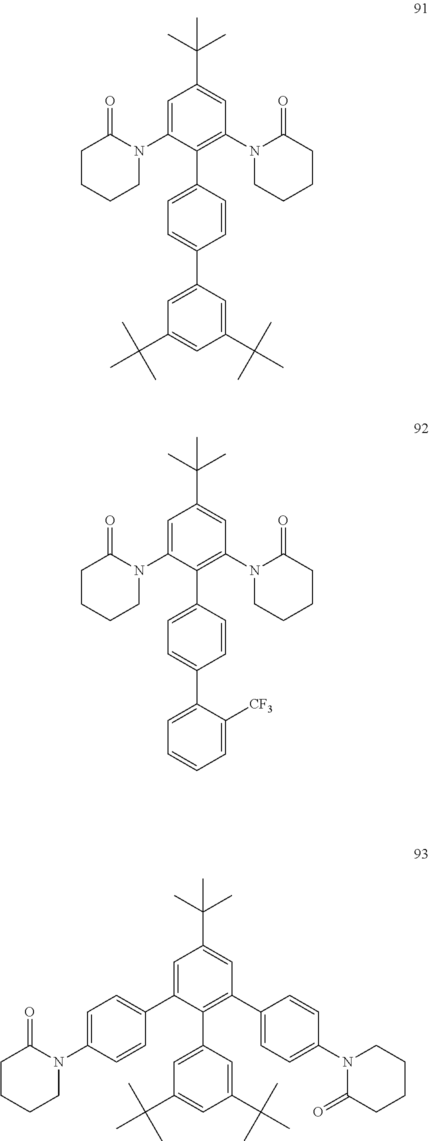 [SMILES: CC(C)(C)c1cc(-c2ccc(-c3c(N4CCCCC4=O)cc(C(C)(C)C)cc3N3CCCCC3=O)cc2)cc(C(C)(C)C)c1 ; CC(C)(C)c1cc(N2CCCCC2=O)c(-c2ccc(-c3ccccc3C(F)(F)F)cc2)c(N2CCCCC2=O)c1 ; CC(C)(C)c1cc(-c2c(-c3ccc(N4CCCCC4=O)cc3)cc(C(C)(C)C)cc2-c2ccc(N3CCCCC3=O)cc2)cc(C(C)(C)C)c1]

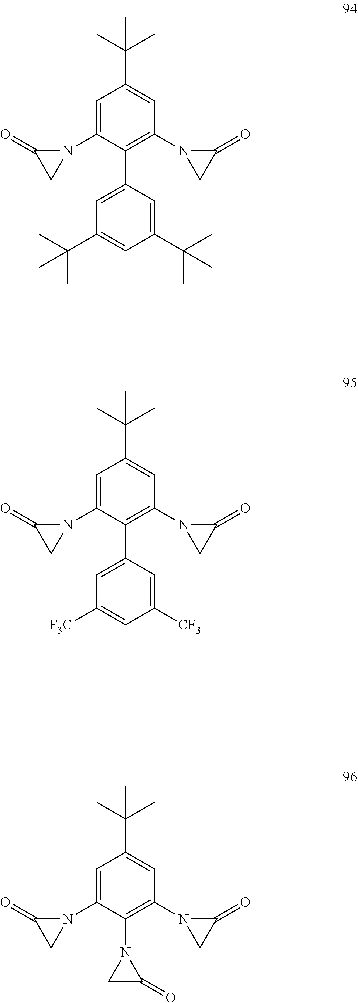 [SMILES: CC(C)(C)c1cc(-c2c(N3CC3=O)cc(C(C)(C)C)cc2N2CC2=O)cc(C(C)(C)C)c1 ; CC(C)(C)c1cc(N2CC2=O)c(-c2cc(C(F)(F)F)cc(C(F)(F)F)c2)c(N2CC2=O)c1 ; CC(C)(C)c1cc(N2CC2=O)c(N2CC2=O)c(N2CC2=O)c1]

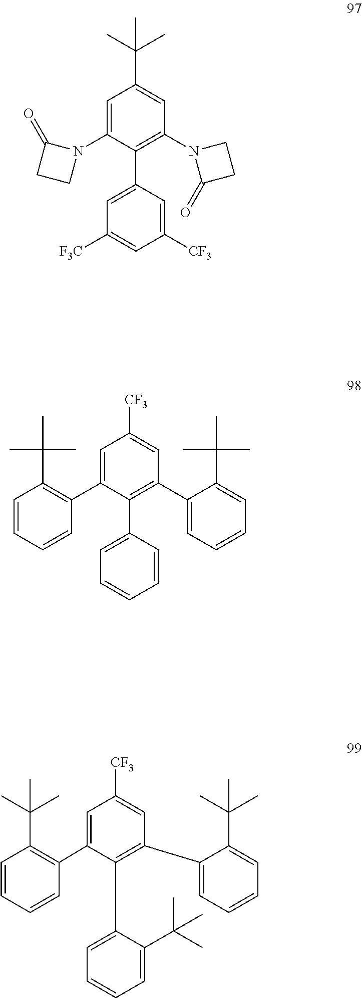 [SMILES: CC(C)(C)c1cc(N2CCC2=O)c(-c2cc(C(F)(F)F)cc(C(F)(F)F)c2)c(N2CCC2=O)c1 ; CC(C)(C)c1ccccc1-c1cc(C(F)(F)F)cc(-c2ccccc2C(C)(C)C)c1-c1ccccc1 ; CC(C)(C)c1ccccc1-c1cc(C(F)(F)F)cc(-c2ccccc2C(C)(C)C)c1-c1ccccc1C(C)(C)C]

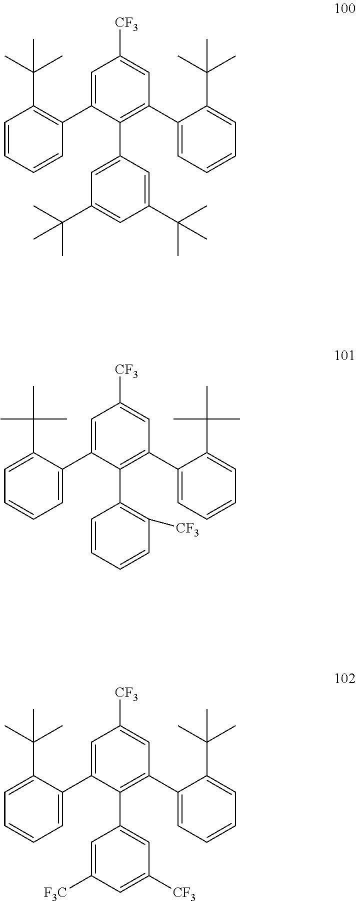

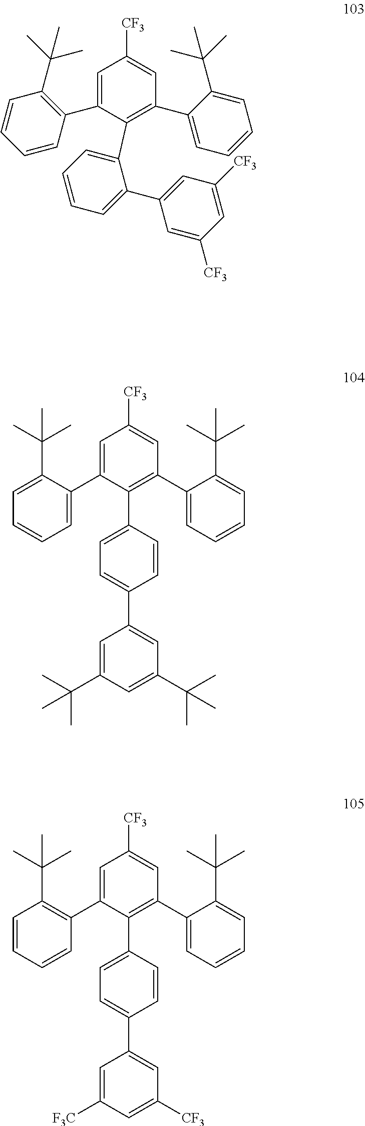 [SMILES: CC(C)(C)c1ccccc1-c1cc(C(F)(F)F)cc(-c2ccccc2C(C)(C)C)c1-c1ccccc1-c1cc(C(F)(F)F)cc(C(F)(F)F)c1 ; CC(C)(C)c1cc(-c2ccc(-c3c(-c4ccccc4C(C)(C)C)cc(C(F)(F)F)cc3-c3ccccc3C(C)(C)C)cc2)cc(C(C)(C)C)c1 ; CC(C)(C)c1ccccc1-c1cc(C(F)(F)F)cc(-c2ccccc2C(C)(C)C)c1-c1ccc(-c2cc(C(F)(F)F)cc(C(F)(F)F)c2)cc1]

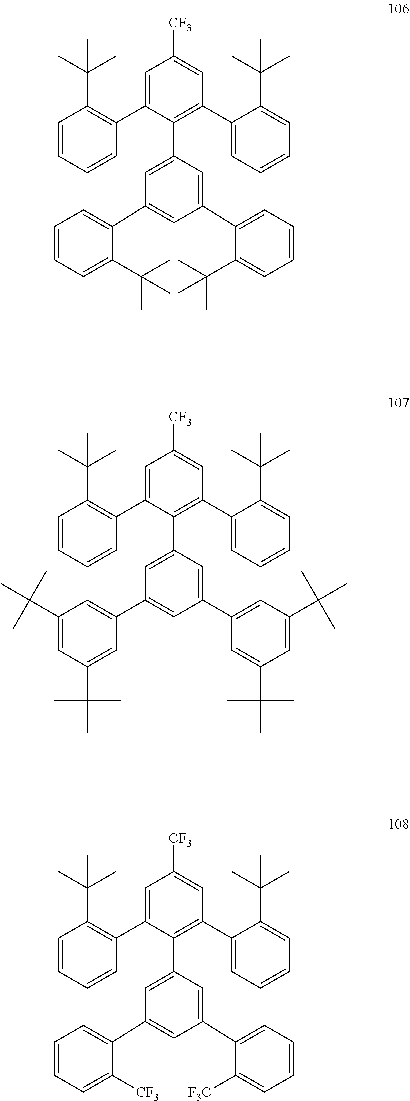 [SMILES: CC(C)(C)c1ccccc1-c1cc(-c2ccccc2C(C)(C)C)cc(-c2c(-c3ccccc3C(C)(C)C)cc(C(F)(F)F)cc2-c2ccccc2C(C)(C)C)c1 ; CC(C)(C)c1cc(-c2cc(-c3cc(C(C)(C)C)cc(C(C)(C)C)c3)cc(-c3c(-c4ccccc4C(C)(C)C)cc(C(F)(F)F)cc3-c3ccccc3C(C)(C)C)c2)cc(C(C)(C)C)c1 ; CC(C)(C)c1ccccc1-c1cc(C(F)(F)F)cc(-c2ccccc2C(C)(C)C)c1-c1cc(-c2ccccc2C(F)(F)F)cc(-c2ccccc2C(F)(F)F)c1]

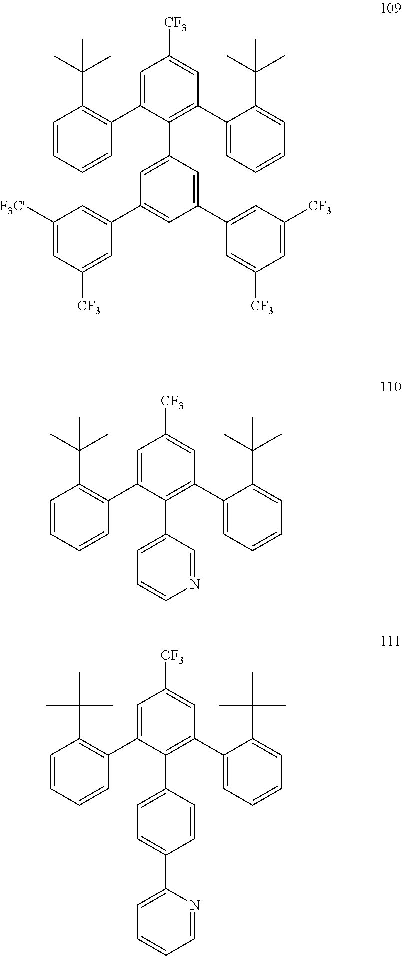

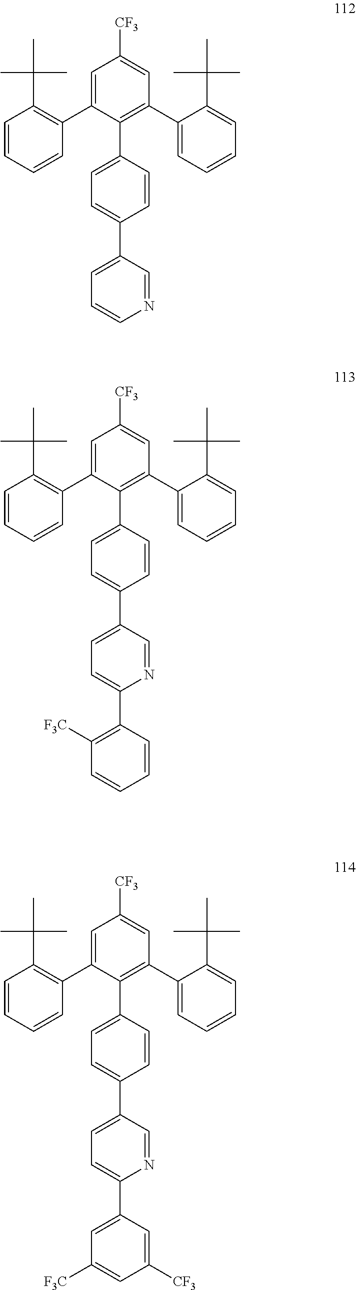

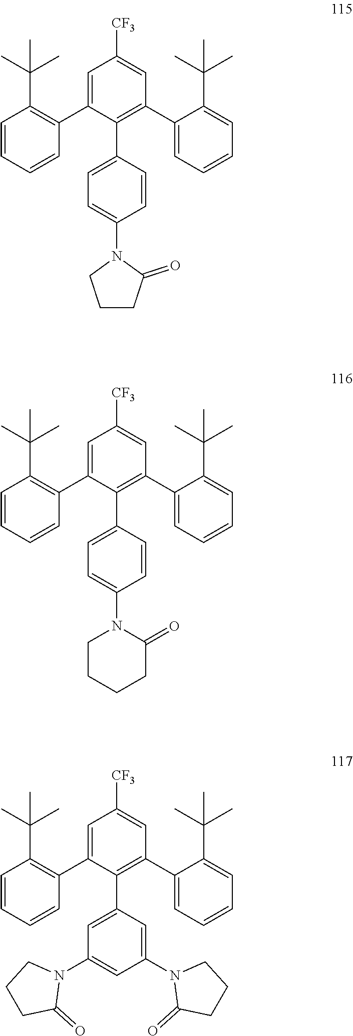 [SMILES: CC(C)(C)c1ccccc1-c1cc(C(F)(F)F)cc(-c2ccccc2C(C)(C)C)c1-c1ccc(N2CCCC2=O)cc1 ; CC(C)(C)c1ccccc1-c1cc(C(F)(F)F)cc(-c2ccccc2C(C)(C)C)c1-c1ccc(N2CCCCC2=O)cc1 ; CC(C)(C)c1ccccc1-c1cc(C(F)(F)F)cc(-c2ccccc2C(C)(C)C)c1-c1cc(N2CCCC2=O)cc(N2CCCC2=O)c1]

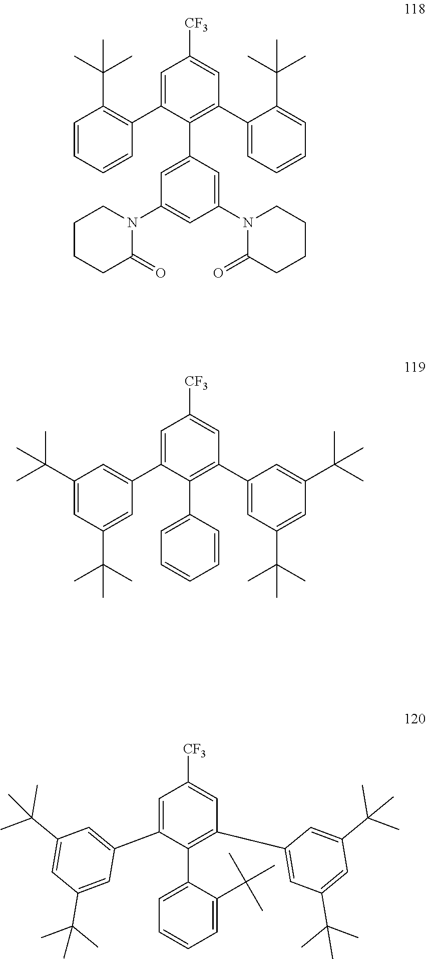

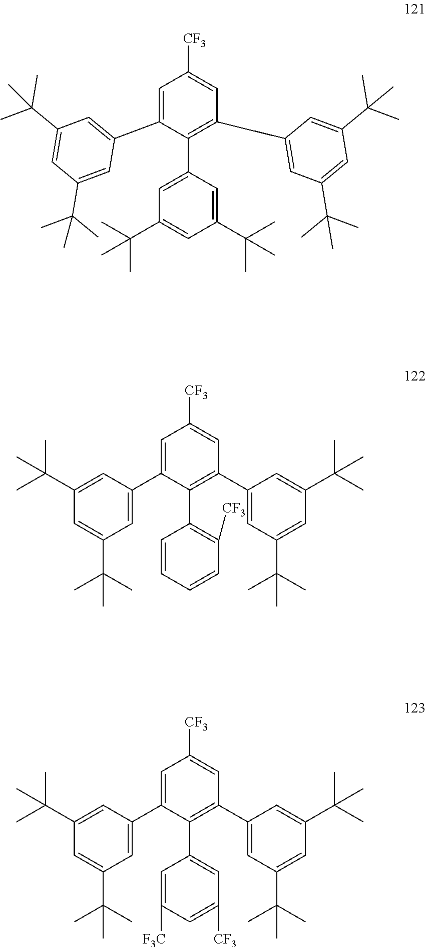

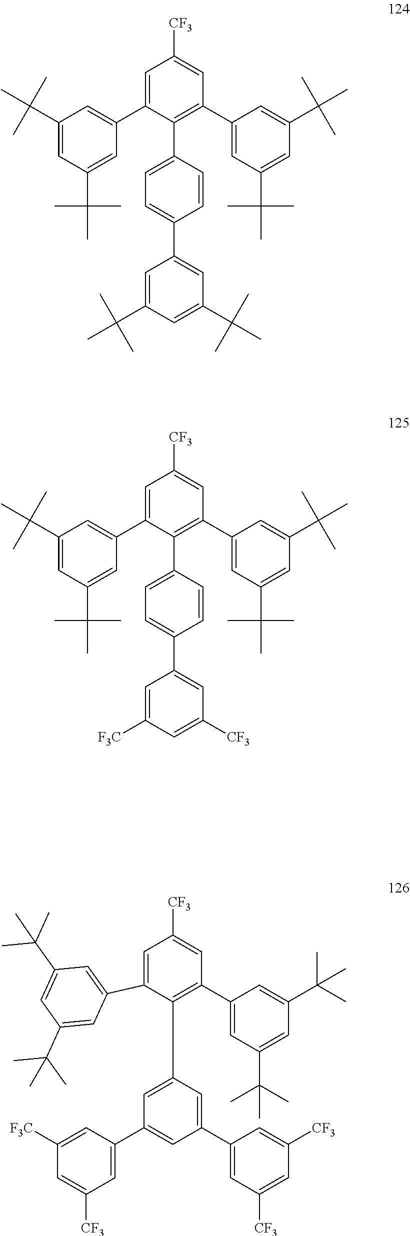

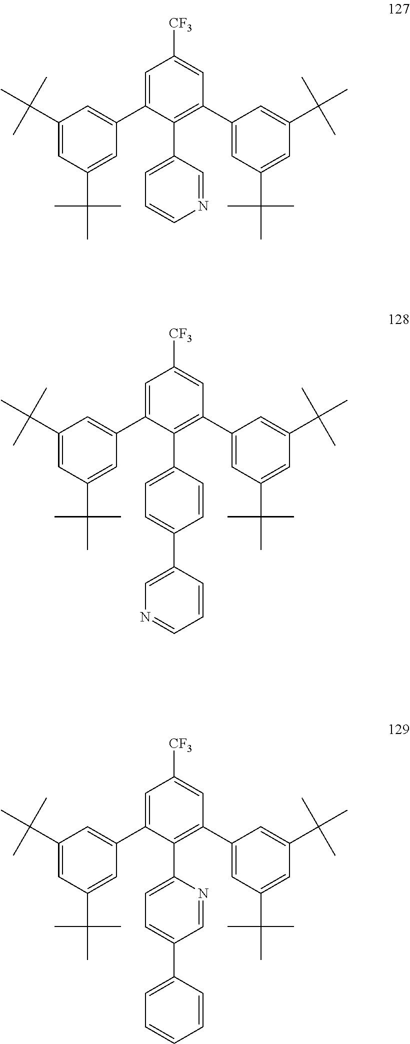

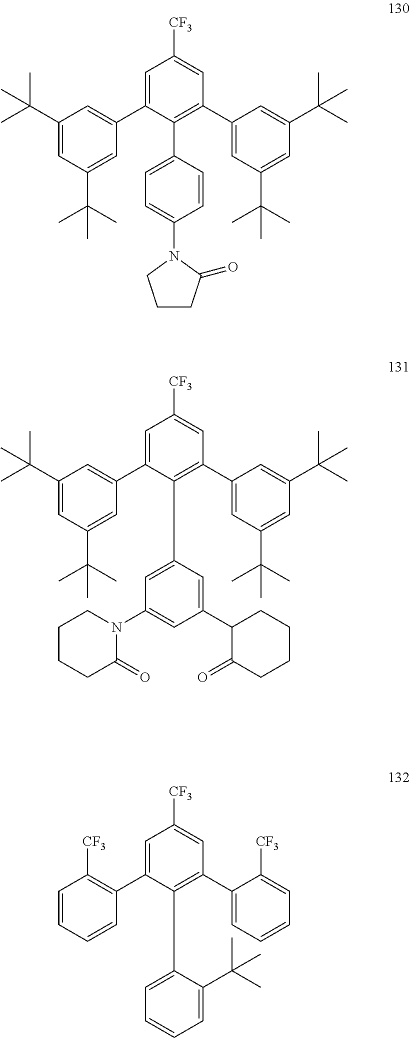

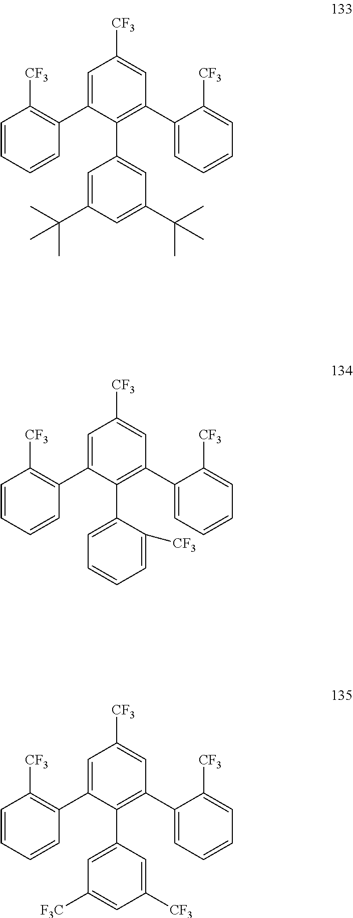 [SMILES: CC(C)(C)c1cc(-c2c(-c3ccccc3C(F)(F)F)cc(C(F)(F)F)cc2-c2ccccc2C(F)(F)F)cc(C(C)(C)C)c1 ; FC(F)(F)c1cc(-c2ccccc2C(F)(F)F)c(-c2ccccc2C(F)(F)F)c(-c2ccccc2C(F)(F)F)c1 ; FC(F)(F)c1cc(-c2c(-c3ccccc3C(F)(F)F)cc(C(F)(F)F)cc2-c2ccccc2C(F)(F)F)cc(C(F)(F)F)c1]

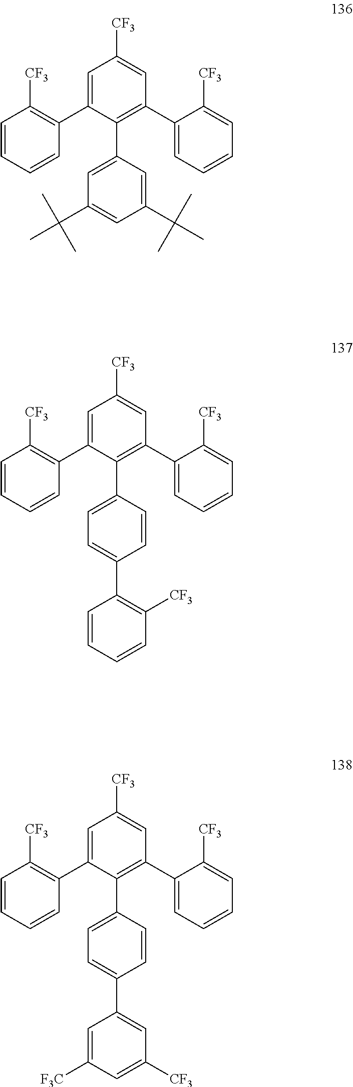 [SMILES: CC(C)(C)c1cc(-c2c(-c3ccccc3C(F)(F)F)cc(C(F)(F)F)cc2-c2ccccc2C(F)(F)F)cc(C(C)(C)C)c1 ; FC(F)(F)c1cc(-c2ccccc2C(F)(F)F)c(-c2ccc(-c3ccccc3C(F)(F)F)cc2)c(-c2ccccc2C(F)(F)F)c1 ; FC(F)(F)c1cc(-c2ccc(-c3c(-c4ccccc4C(F)(F)F)cc(C(F)(F)F)cc3-c3ccccc3C(F)(F)F)cc2)cc(C(F)(F)F)c1]

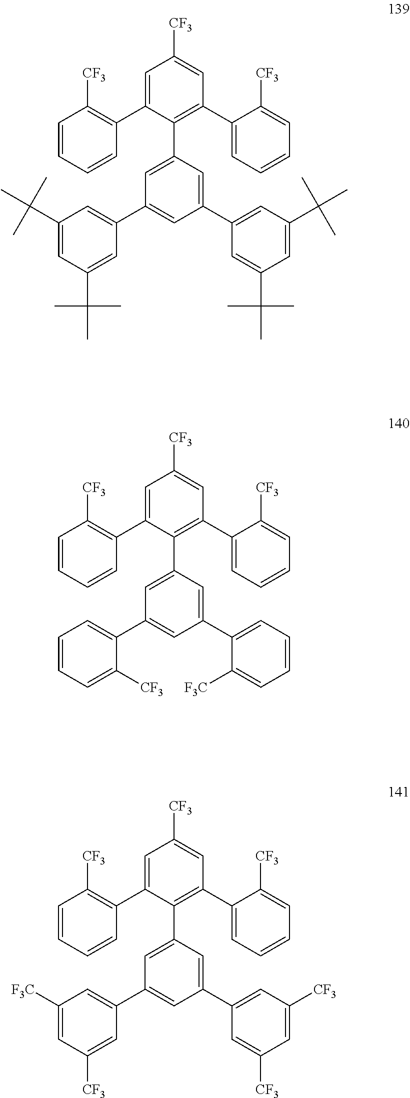 [SMILES: CC(C)(C)c1cc(-c2cc(-c3cc(C(C)(C)C)cc(C(C)(C)C)c3)cc(-c3c(-c4ccccc4C(F)(F)F)cc(C(F)(F)F)cc3-c3ccccc3C(F)(F)F)c2)cc(C(C)(C)C)c1 ; FC(F)(F)c1cc(-c2ccccc2C(F)(F)F)c(-c2cc(-c3ccccc3C(F)(F)F)cc(-c3ccccc3C(F)(F)F)c2)c(-c2ccccc2C(F)(F)F)c1 ; FC(F)(F)c1cc(-c2cc(-c3cc(C(F)(F)F)cc(C(F)(F)F)c3)cc(-c3c(-c4ccccc4C(F)(F)F)cc(C(F)(F)F)cc3-c3ccccc3C(F)(F)F)c2)cc(C(F)(F)F)c1]

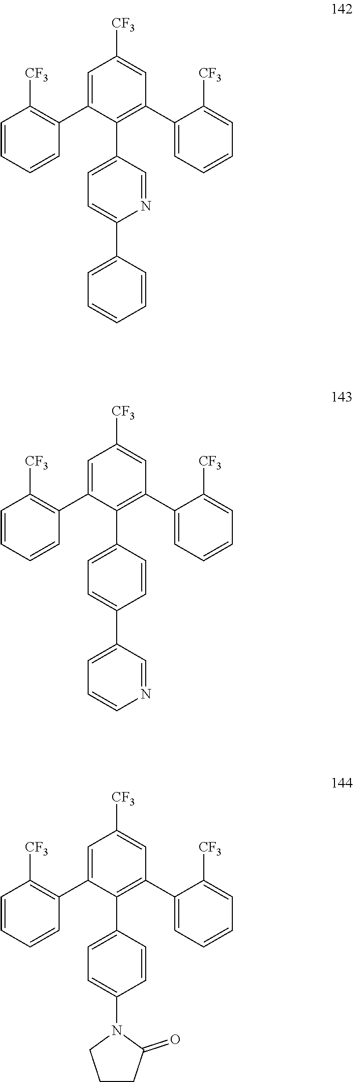 [SMILES: FC(F)(F)c1cc(-c2ccccc2C(F)(F)F)c(-c2ccc(-c3ccccc3)nc2)c(-c2ccccc2C(F)(F)F)c1 ; FC(F)(F)c1cc(-c2ccccc2C(F)(F)F)c(-c2ccc(-c3cccnc3)cc2)c(-c2ccccc2C(F)(F)F)c1 ; O=C1CCCN1c1ccc(-c2c(-c3ccccc3C(F)(F)F)cc(C(F)(F)F)cc2-c2ccccc2C(F)(F)F)cc1]

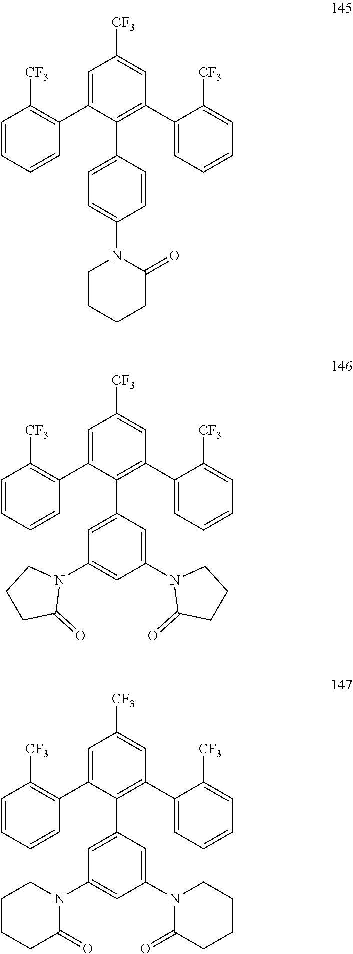 [SMILES: O=C1CCCCN1c1ccc(-c2c(-c3ccccc3C(F)(F)F)cc(C(F)(F)F)cc2-c2ccccc2C(F)(F)F)cc1 ; O=C1CCCN1c1cc(-c2c(-c3ccccc3C(F)(F)F)cc(C(F)(F)F)cc2-c2ccccc2C(F)(F)F)cc(N2CCCC2=O)c1 ; O=C1CCCCN1c1cc(-c2c(-c3ccccc3C(F)(F)F)cc(C(F)(F)F)cc2-c2ccccc2C(F)(F)F)cc(N2CCCCC2=O)c1]

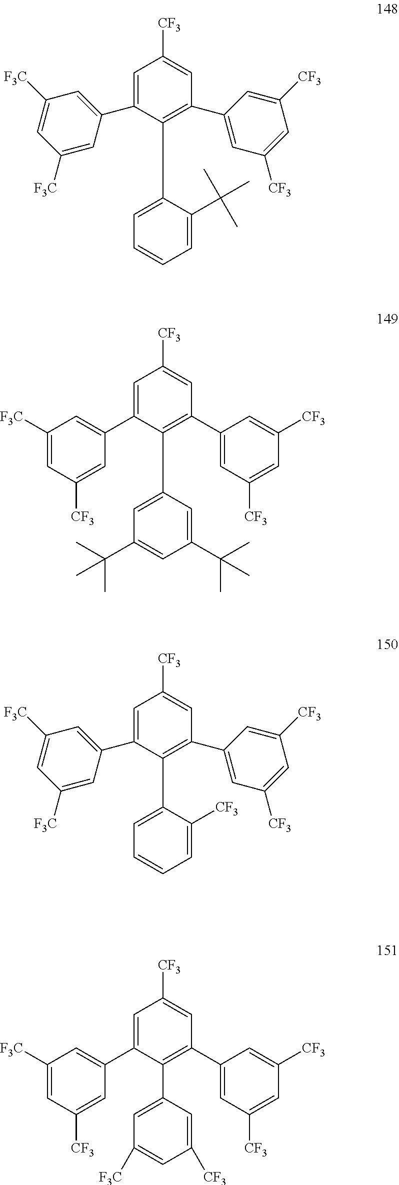 [SMILES: CC(C)(C)c1ccccc1-c1c(-c2cc(C(F)(F)F)cc(C(F)(F)F)c2)cc(C(F)(F)F)cc1-c1cc(C(F)(F)F)cc(C(F)(F)F)c1 ; CC(C)(C)c1cc(-c2c(-c3cc(C(F)(F)F)cc(C(F)(F)F)c3)cc(C(F)(F)F)cc2-c2cc(C(F)(F)F)cc(C(F)(F)F)c2)cc(C(C)(C)C)c1 ; FC(F)(F)c1cc(-c2cc(C(F)(F)F)cc(-c3cc(C(F)(F)F)cc(C(F)(F)F)c3)c2-c2ccccc2C(F)(F)F)cc(C(F)(F)F)c1 ; FC(F)(F)c1cc(-c2cc(C(F)(F)F)cc(-c3cc(C(F)(F)F)cc(C(F)(F)F)c3)c2-c2cc(C(F)(F)F)cc(C(F)(F)F)c2)cc(C(F)(F)F)c1]

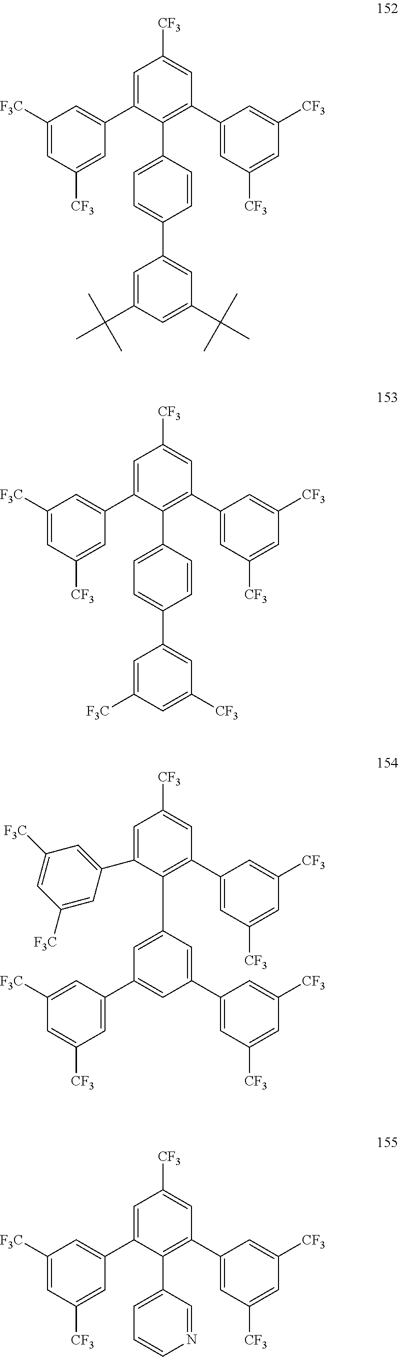 [SMILES: CC(C)(C)c1cc(-c2ccc(-c3c(-c4cc(C(F)(F)F)cc(C(F)(F)F)c4)cc(C(F)(F)F)cc3-c3cc(C(F)(F)F)cc(C(F)(F)F)c3)cc2)cc(C(C)(C)C)c1 ; FC(F)(F)c1cc(-c2ccc(-c3c(-c4cc(C(F)(F)F)cc(C(F)(F)F)c4)cc(C(F)(F)F)cc3-c3cc(C(F)(F)F)cc(C(F)(F)F)c3)cc2)cc(C(F)(F)F)c1 ; FC(F)(F)c1cc(-c2cc(-c3cc(C(F)(F)F)cc(C(F)(F)F)c3)cc(-c3c(-c4cc(C(F)(F)F)cc(C(F)(F)F)c4)cc(C(F)(F)F)cc3-c3cc(C(F)(F)F)cc(C(F)(F)F)c3)c2)cc(C(F)(F)F)c1 ; FC(F)(F)c1cc(-c2cc(C(F)(F)F)cc(-c3cc(C(F)(F)F)cc(C(F)(F)F)c3)c2-c2cccnc2)cc(C(F)(F)F)c1]

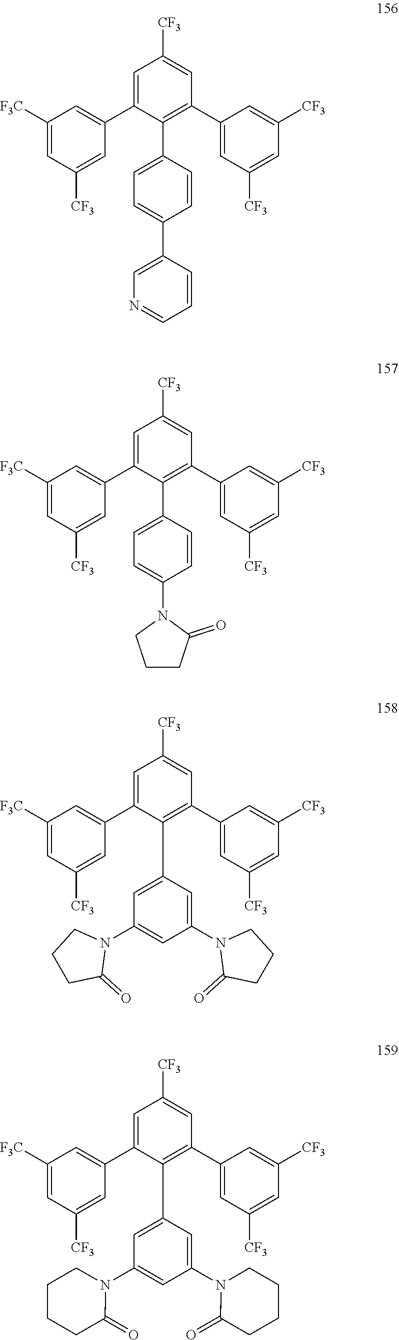 [SMILES: FC(F)(F)c1cc(-c2cc(C(F)(F)F)cc(-c3cc(C(F)(F)F)cc(C(F)(F)F)c3)c2-c2ccc(-c3cccnc3)cc2)cc(C(F)(F)F)c1 ; O=C1CCCN1c1ccc(-c2c(-c3cc(C(F)(F)F)cc(C(F)(F)F)c3)cc(C(F)(F)F)cc2-c2cc(C(F)(F)F)cc(C(F)(F)F)c2)cc1 ; O=C1CCCN1c1cc(-c2c(-c3cc(C(F)(F)F)cc(C(F)(F)F)c3)cc(C(F)(F)F)cc2-c2cc(C(F)(F)F)cc(C(F)(F)F)c2)cc(N2CCCC2=O)c1 ; O=C1CCCCN1c1cc(-c2c(-c3cc(C(F)(F)F)cc(C(F)(F)F)c3)cc(C(F)(F)F)cc2-c2cc(C(F)(F)F)cc(C(F)(F)F)c2)cc(N2CCCCC2=O)c1]

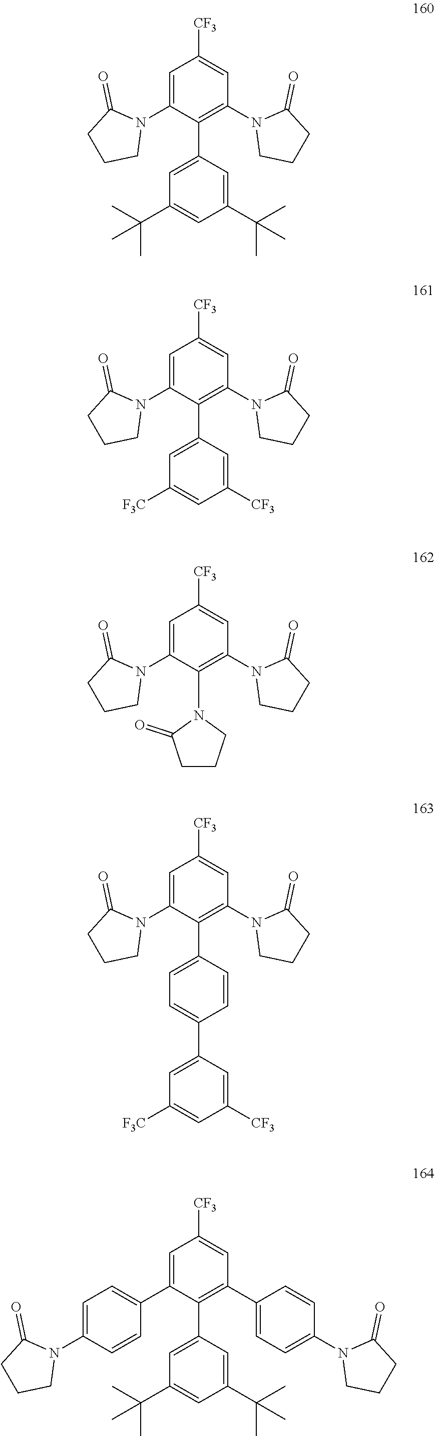 [SMILES: CC(C)(C)c1cc(-c2c(N3CCCC3=O)cc(C(F)(F)F)cc2N2CCCC2=O)cc(C(C)(C)C)c1 ; O=C1CCCN1c1cc(C(F)(F)F)cc(N2CCCC2=O)c1-c1cc(C(F)(F)F)cc(C(F)(F)F)c1 ; O=C1CCCN1c1cc(C(F)(F)F)cc(N2CCCC2=O)c1N1CCCC1=O ; O=C1CCCN1c1cc(C(F)(F)F)cc(N2CCCC2=O)c1-c1ccc(-c2cc(C(F)(F)F)cc(C(F)(F)F)c2)cc1 ; CC(C)(C)c1cc(-c2c(-c3ccc(N4CCCC4=O)cc3)cc(C(F)(F)F)cc2-c2ccc(N3CCCC3=O)cc2)cc(C(C)(C)C)c1]

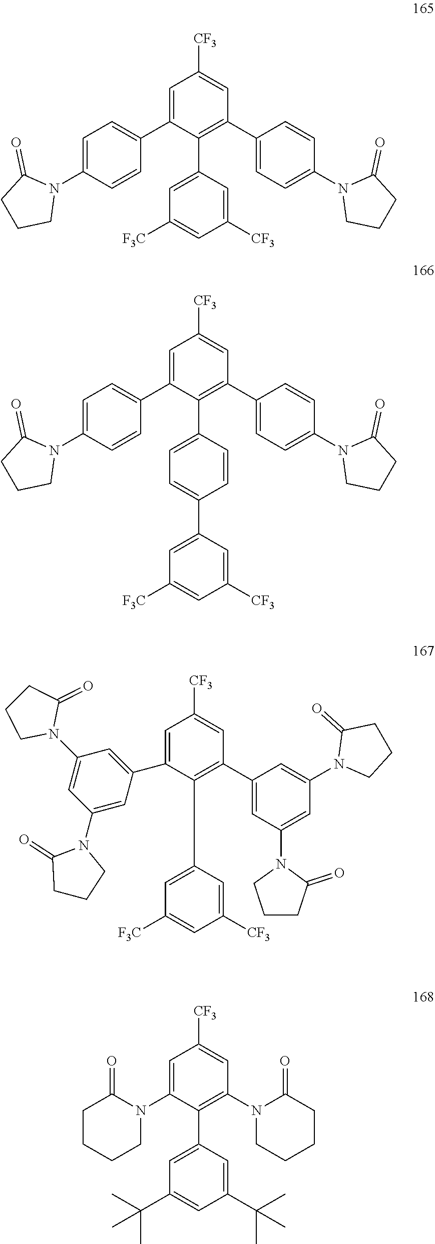 [SMILES: O=C1CCCN1c1ccc(-c2cc(C(F)(F)F)cc(-c3ccc(N4CCCC4=O)cc3)c2-c2cc(C(F)(F)F)cc(C(F)(F)F)c2)cc1 ; O=C1CCCN1c1ccc(-c2cc(C(F)(F)F)cc(-c3ccc(N4CCCC4=O)cc3)c2-c2ccc(-c3cc(C(F)(F)F)cc(C(F)(F)F)c3)cc2)cc1 ; O=C1CCCN1c1cc(-c2cc(C(F)(F)F)cc(-c3cc(N4CCCC4=O)cc(N4CCCC4=O)c3)c2-c2cc(C(F)(F)F)cc(C(F)(F)F)c2)cc(N2CCCC2=O)c1 ; CC(C)(C)c1cc(-c2c(N3CCCCC3=O)cc(C(F)(F)F)cc2N2CCCCC2=O)cc(C(C)(C)C)c1]

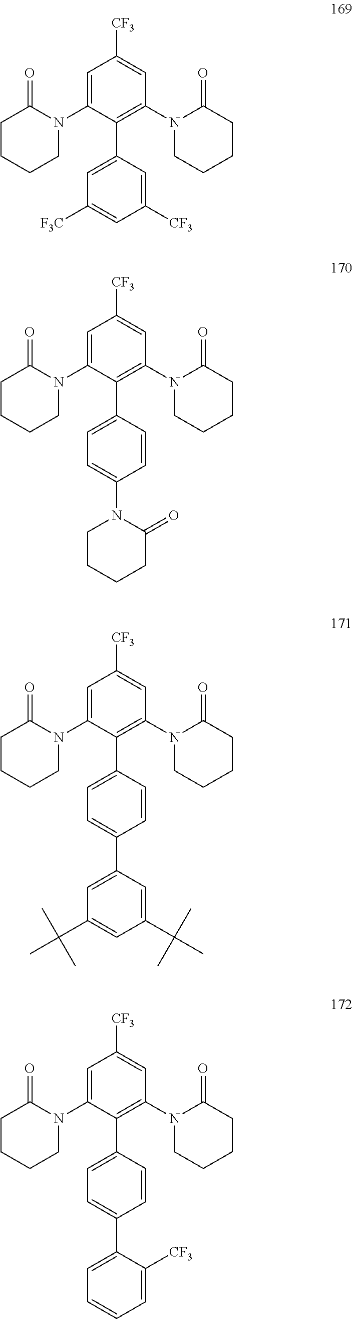 [SMILES: O=C1CCCCN1c1cc(C(F)(F)F)cc(N2CCCCC2=O)c1-c1cc(C(F)(F)F)cc(C(F)(F)F)c1 ; O=C1CCCCN1c1ccc(-c2c(N3CCCCC3=O)cc(C(F)(F)F)cc2N2CCCCC2=O)cc1 ; CC(C)(C)c1cc(-c2ccc(-c3c(N4CCCCC4=O)cc(C(F)(F)F)cc3N3CCCCC3=O)cc2)cc(C(C)(C)C)c1 ; O=C1CCCCN1c1cc(C(F)(F)F)cc(N2CCCCC2=O)c1-c1ccc(-c2ccccc2C(F)(F)F)cc1]

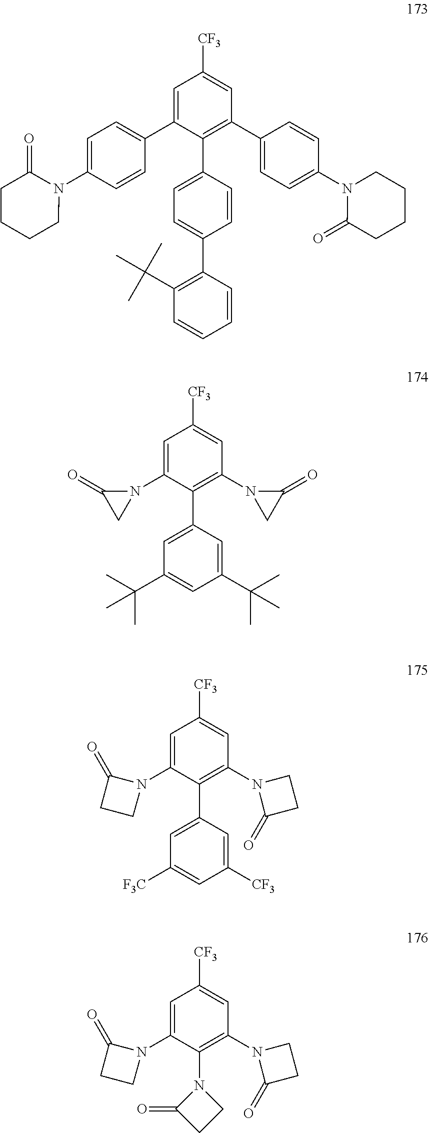 [SMILES: CC(C)(C)c1ccccc1-c1ccc(-c2c(-c3ccc(N4CCCCC4=O)cc3)cc(C(F)(F)F)cc2-c2ccc(N3CCCCC3=O)cc2)cc1 ; CC(C)(C)c1cc(-c2c(N3CC3=O)cc(C(F)(F)F)cc2N2CC2=O)cc(C(C)(C)C)c1 ; O=C1CCN1c1cc(C(F)(F)F)cc(N2CCC2=O)c1-c1cc(C(F)(F)F)cc(C(F)(F)F)c1 ; O=C1CCN1c1cc(C(F)(F)F)cc(N2CCC2=O)c1N1CCC1=O]

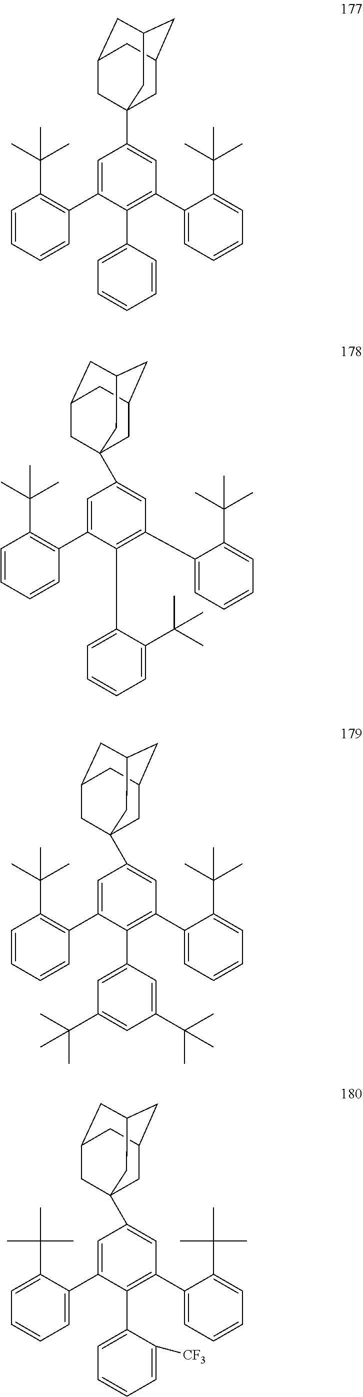

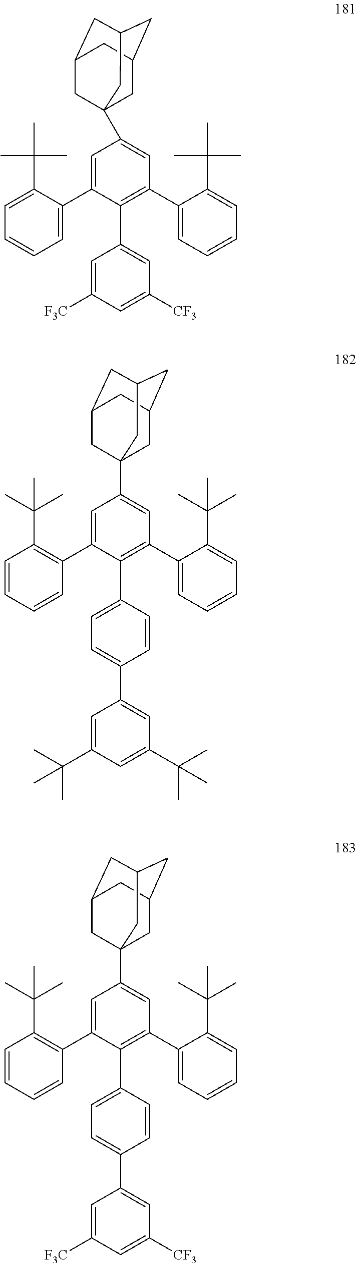

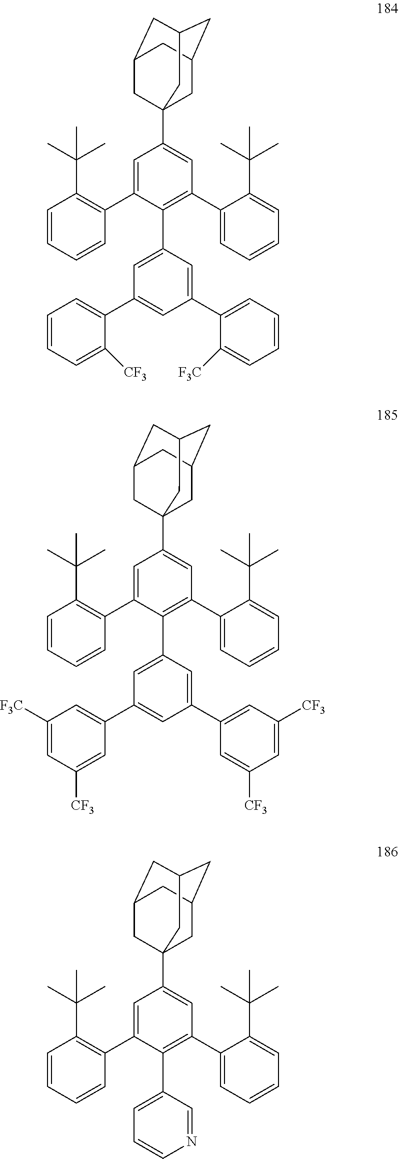 [SMILES: CC(C)(C)c1ccccc1-c1cc(C23CC4CC(CC(C4)C2)C3)cc(-c2ccccc2C(C)(C)C)c1-c1cc(-c2ccccc2C(F)(F)F)cc(-c2ccccc2C(F)(F)F)c1 ; CC(C)(C)c1ccccc1-c1cc(C23CC4CC(CC(C4)C2)C3)cc(-c2ccccc2C(C)(C)C)c1-c1cc(-c2cc(C(F)(F)F)cc(C(F)(F)F)c2)cc(-c2cc(C(F)(F)F)cc(C(F)(F)F)c2)c1 ; CC(C)(C)c1ccccc1-c1cc(C23CC4CC(CC(C4)C2)C3)cc(-c2ccccc2C(C)(C)C)c1-c1cccnc1]

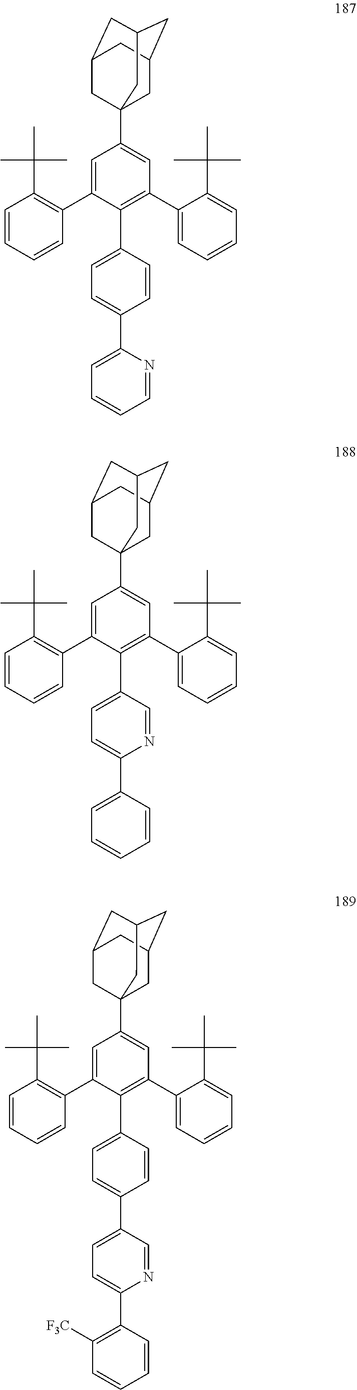

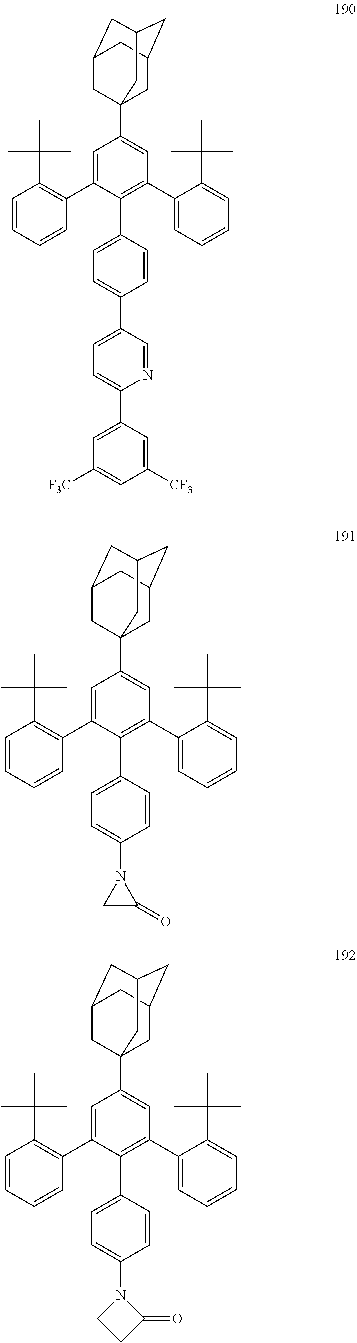

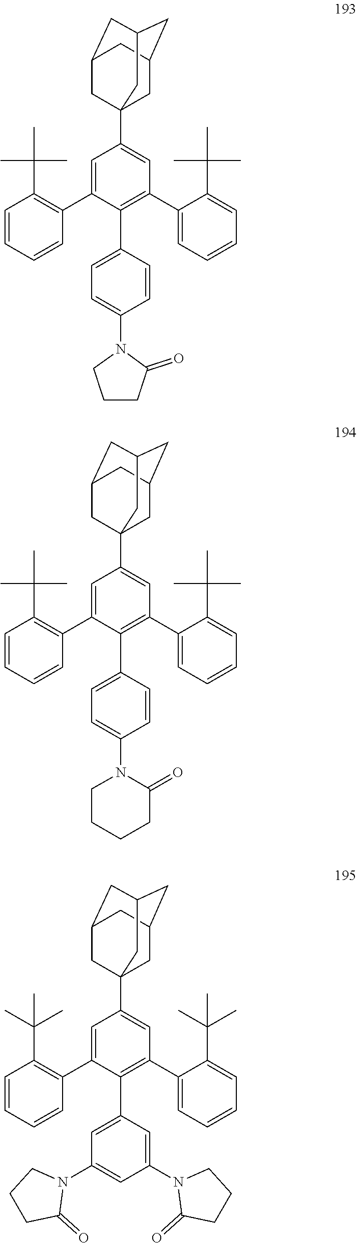

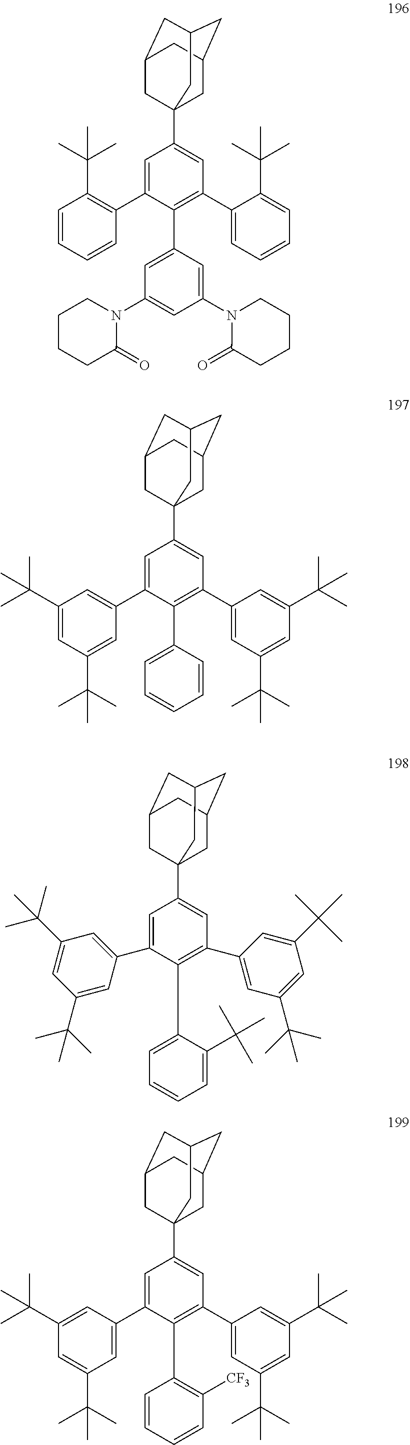

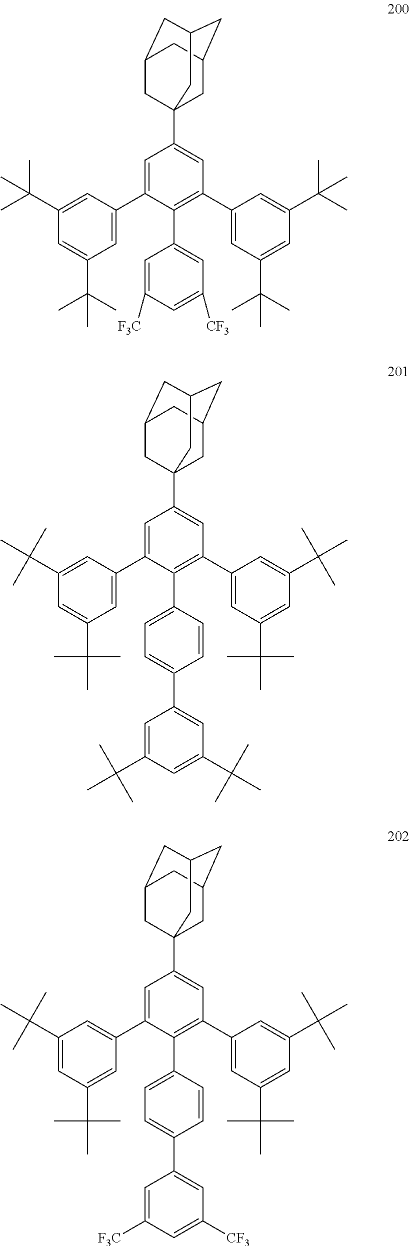

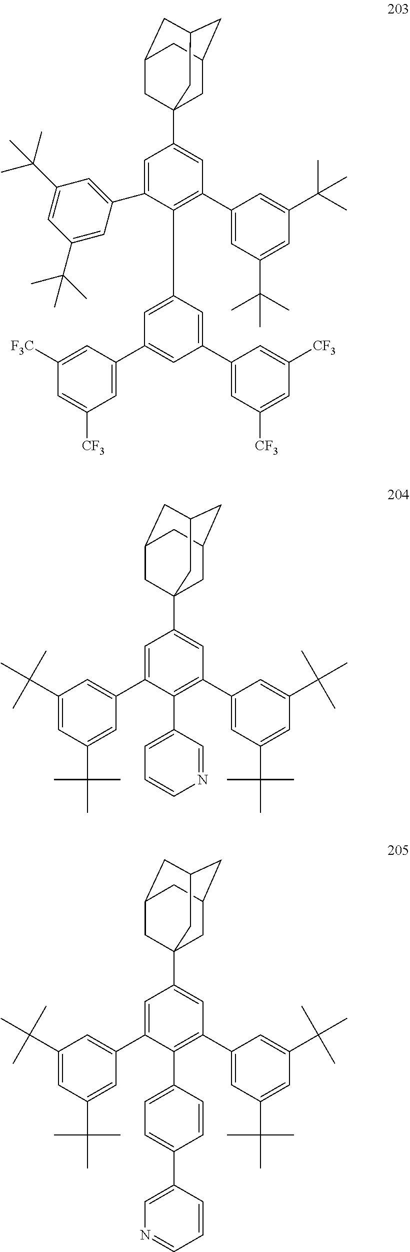

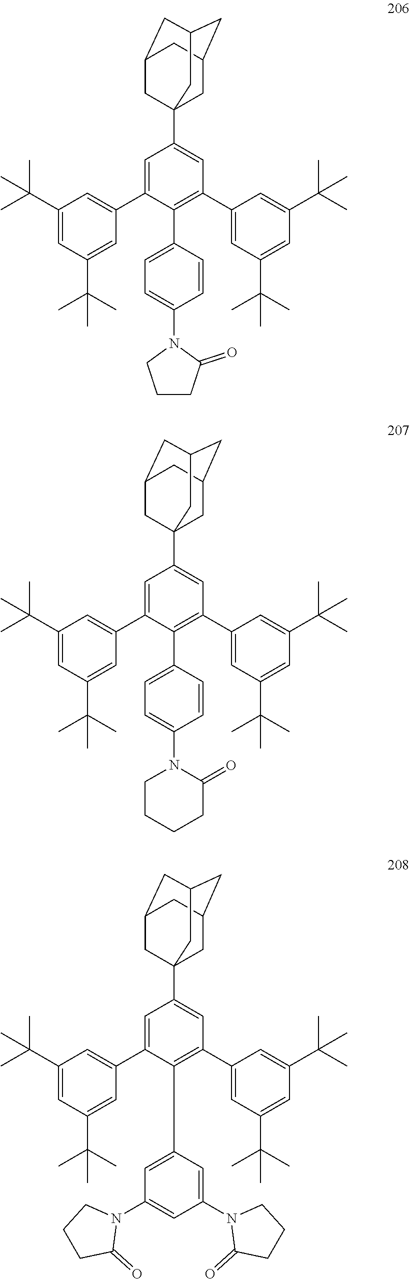

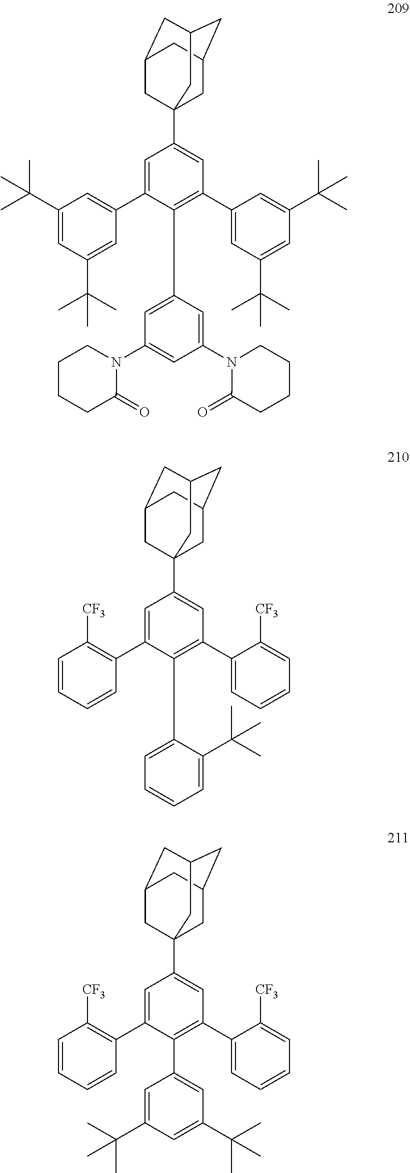

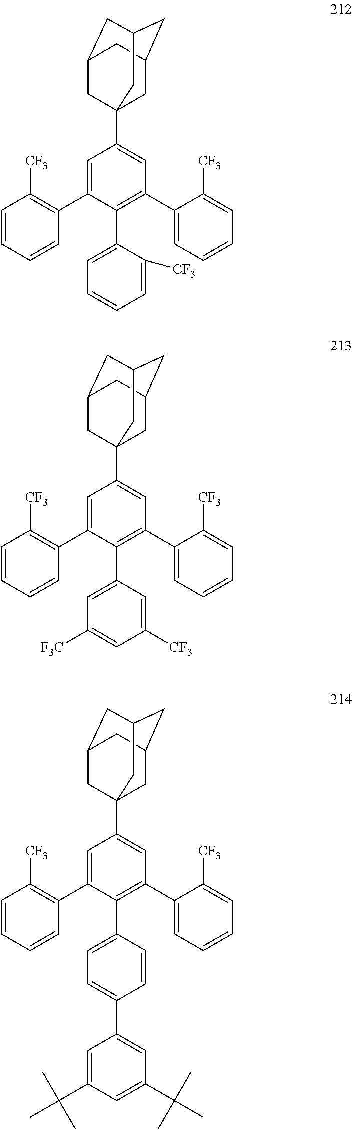

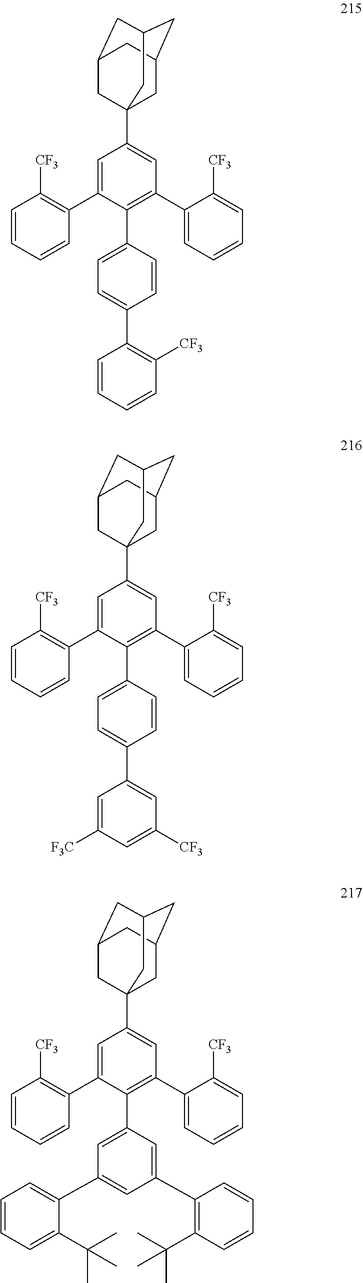

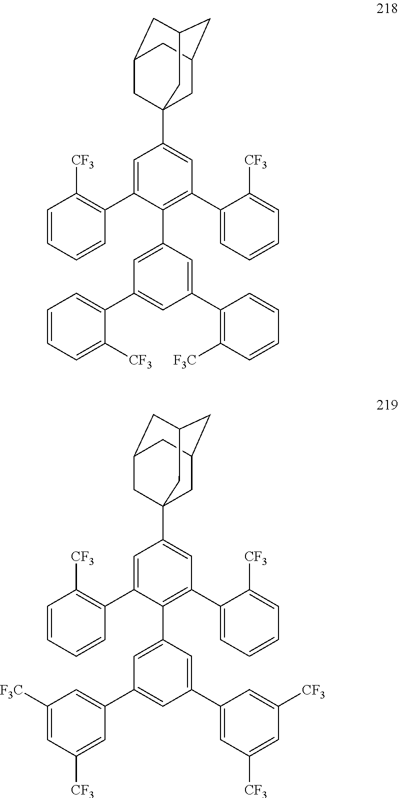 [SMILES: FC(F)(F)c1ccccc1-c1cc(-c2ccccc2C(F)(F)F)cc(-c2c(-c3ccccc3C(F)(F)F)cc(C34CC5CC(CC(C5)C3)C4)cc2-c2ccccc2C(F)(F)F)c1 ; FC(F)(F)c1cc(-c2cc(-c3cc(C(F)(F)F)cc(C(F)(F)F)c3)cc(-c3c(-c4ccccc4C(F)(F)F)cc(C45CC6CC(CC(C6)C4)C5)cc3-c3ccccc3C(F)(F)F)c2)cc(C(F)(F)F)c1]

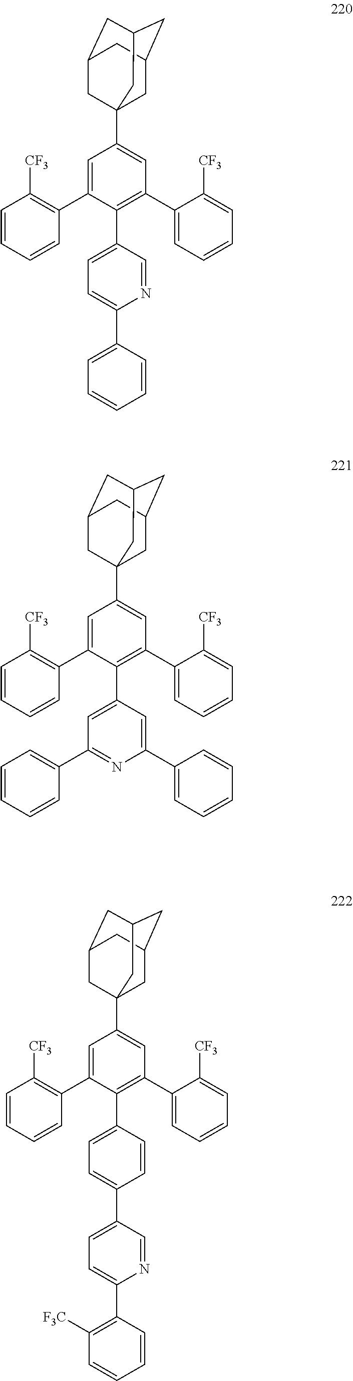 [SMILES: FC(F)(F)c1ccccc1-c1cc(C23CC4CC(CC(C4)C2)C3)cc(-c2ccccc2C(F)(F)F)c1-c1ccc(-c2ccccc2)nc1 ; FC(F)(F)c1ccccc1-c1cc(C23CC4CC(CC(C4)C2)C3)cc(-c2ccccc2C(F)(F)F)c1-c1cc(-c2ccccc2)nc(-c2ccccc2)c1 ; FC(F)(F)c1ccccc1-c1ccc(-c2ccc(-c3c(-c4ccccc4C(F)(F)F)cc(C45CC6CC(CC(C6)C4)C5)cc3-c3ccccc3C(F)(F)F)cc2)cn1]

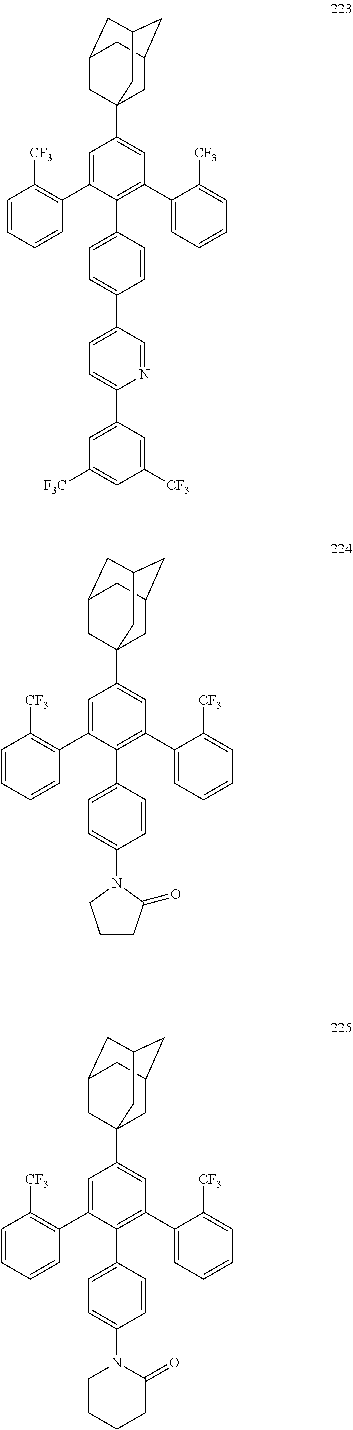 [SMILES: FC(F)(F)c1cc(-c2ccc(-c3ccc(-c4c(-c5ccccc5C(F)(F)F)cc(C56CC7CC(CC(C7)C5)C6)cc4-c4ccccc4C(F)(F)F)cc3)cn2)cc(C(F)(F)F)c1 ; O=C1CCCN1c1ccc(-c2c(-c3ccccc3C(F)(F)F)cc(C34CC5CC(CC(C5)C3)C4)cc2-c2ccccc2C(F)(F)F)cc1 ; O=C1CCCCN1c1ccc(-c2c(-c3ccccc3C(F)(F)F)cc(C34CC5CC(CC(C5)C3)C4)cc2-c2ccccc2C(F)(F)F)cc1]

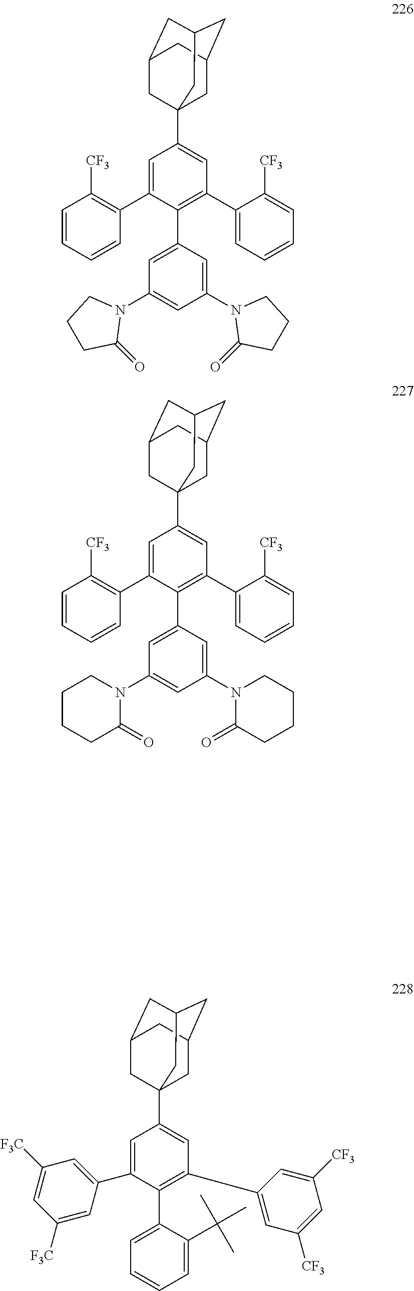

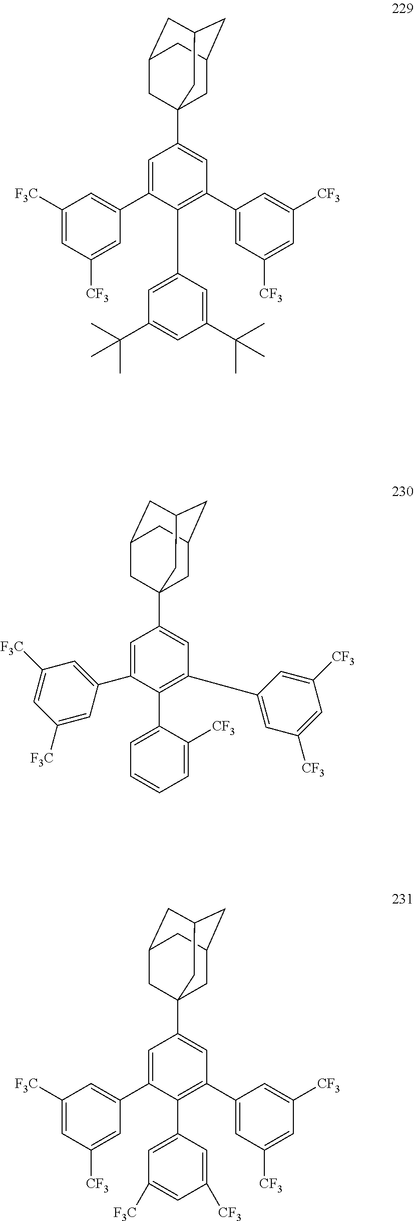 [SMILES: CC(C)(C)c1cc(-c2c(-c3cc(C(F)(F)F)cc(C(F)(F)F)c3)cc(C34CC5CC(CC(C5)C3)C4)cc2-c2cc(C(F)(F)F)cc(C(F)(F)F)c2)cc(C(C)(C)C)c1 ; FC(F)(F)c1cc(-c2cc(C34CC5CC(CC(C5)C3)C4)cc(-c3cc(C(F)(F)F)cc(C(F)(F)F)c3)c2-c2ccccc2C(F)(F)F)cc(C(F)(F)F)c1 ; FC(F)(F)c1cc(-c2cc(C34CC5CC(CC(C5)C3)C4)cc(-c3cc(C(F)(F)F)cc(C(F)(F)F)c3)c2-c2cc(C(F)(F)F)cc(C(F)(F)F)c2)cc(C(F)(F)F)c1]

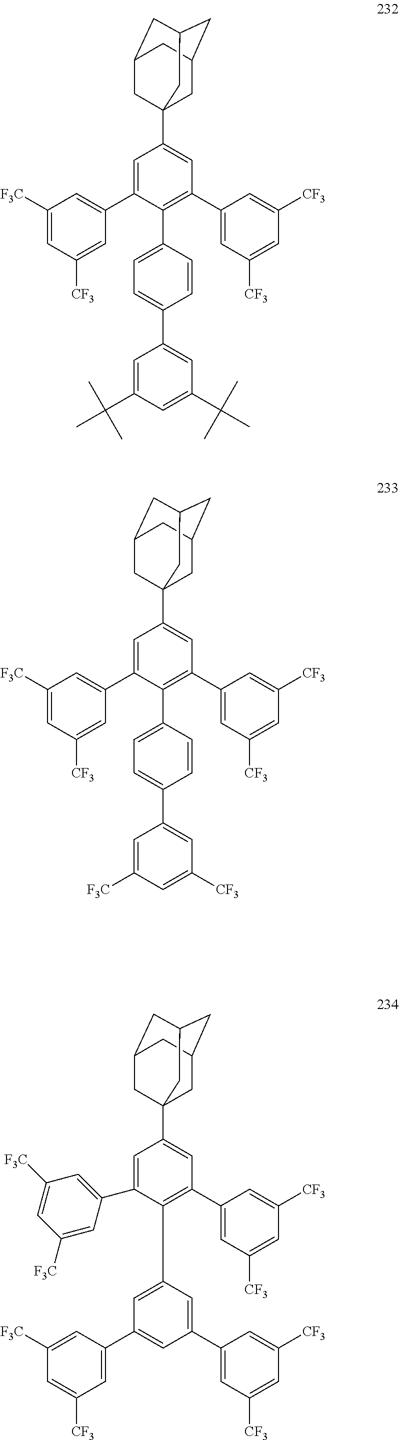 [SMILES: CC(C)(C)c1cc(-c2ccc(-c3c(-c4cc(C(F)(F)F)cc(C(F)(F)F)c4)cc(C45CC6CC(CC(C6)C4)C5)cc3-c3cc(C(F)(F)F)cc(C(F)(F)F)c3)cc2)cc(C(C)(C)C)c1 ; FC(F)(F)c1cc(-c2ccc(-c3c(-c4cc(C(F)(F)F)cc(C(F)(F)F)c4)cc(C45CC6CC(CC(C6)C4)C5)cc3-c3cc(C(F)(F)F)cc(C(F)(F)F)c3)cc2)cc(C(F)(F)F)c1 ; FC(F)(F)c1cc(-c2cc(-c3cc(C(F)(F)F)cc(C(F)(F)F)c3)cc(-c3c(-c4cc(C(F)(F)F)cc(C(F)(F)F)c4)cc(C45CC6CC(CC(C6)C4)C5)cc3-c3cc(C(F)(F)F)cc(C(F)(F)F)c3)c2)cc(C(F)(F)F)c1]

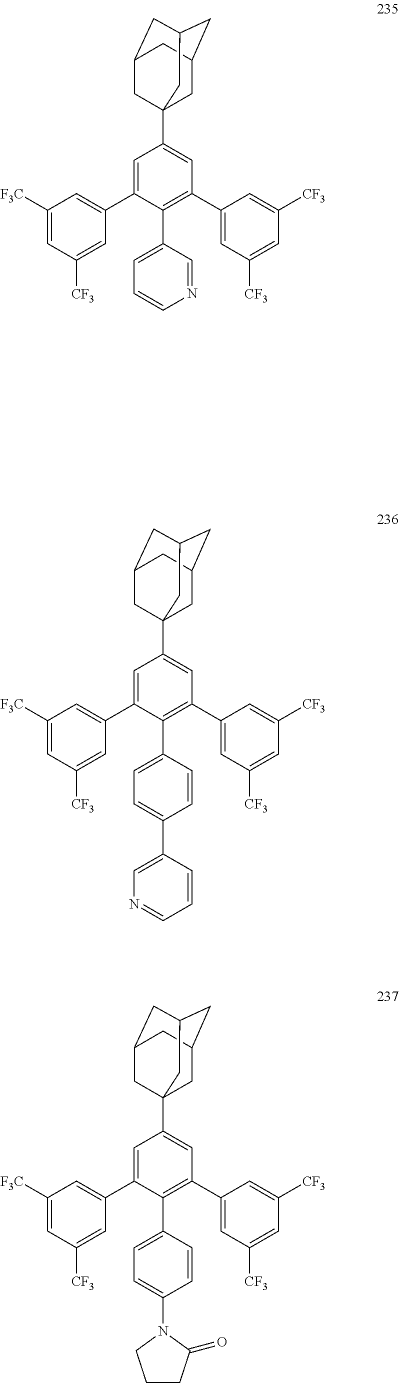 [SMILES: FC(F)(F)c1cc(-c2cc(C34CC5CC(CC(C5)C3)C4)cc(-c3cc(C(F)(F)F)cc(C(F)(F)F)c3)c2-c2cccnc2)cc(C(F)(F)F)c1 ; FC(F)(F)c1cc(-c2cc(C34CC5CC(CC(C5)C3)C4)cc(-c3cc(C(F)(F)F)cc(C(F)(F)F)c3)c2-c2ccc(-c3cccnc3)cc2)cc(C(F)(F)F)c1 ; O=C1CCCN1c1ccc(-c2c(-c3cc(C(F)(F)F)cc(C(F)(F)F)c3)cc(C34CC5CC(CC(C5)C3)C4)cc2-c2cc(C(F)(F)F)cc(C(F)(F)F)c2)cc1]

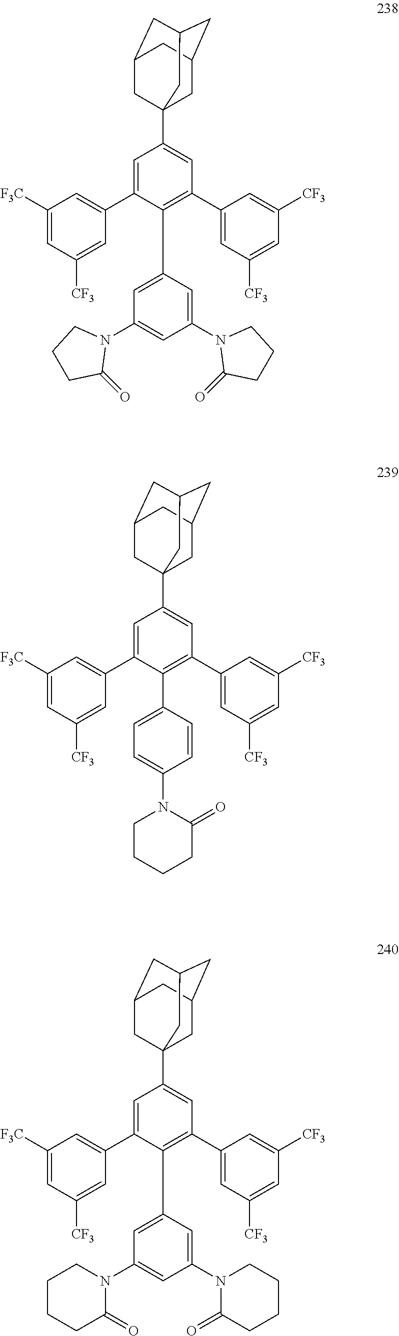 [SMILES: O=C1CCCN1c1cc(-c2c(-c3cc(C(F)(F)F)cc(C(F)(F)F)c3)cc(C34CC5CC(CC(C5)C3)C4)cc2-c2cc(C(F)(F)F)cc(C(F)(F)F)c2)cc(N2CCCC2=O)c1 ; O=C1CCCCN1c1ccc(-c2c(-c3cc(C(F)(F)F)cc(C(F)(F)F)c3)cc(C34CC5CC(CC(C5)C3)C4)cc2-c2cc(C(F)(F)F)cc(C(F)(F)F)c2)cc1 ; O=C1CCCCN1c1cc(-c2c(-c3cc(C(F)(F)F)cc(C(F)(F)F)c3)cc(C34CC5CC(CC(C5)C3)C4)cc2-c2cc(C(F)(F)F)cc(C(F)(F)F)c2)cc(N2CCCCC2=O)c1]

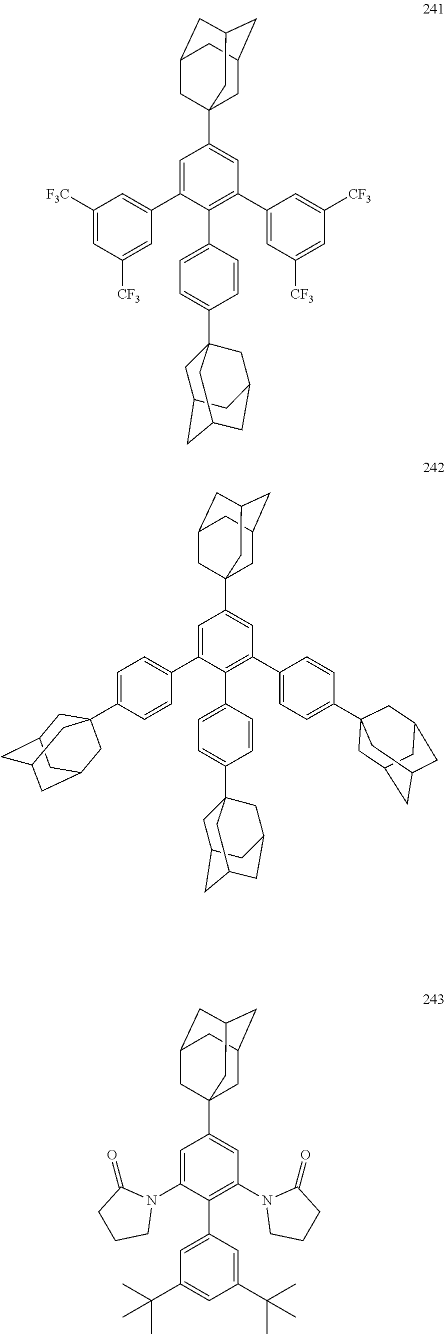

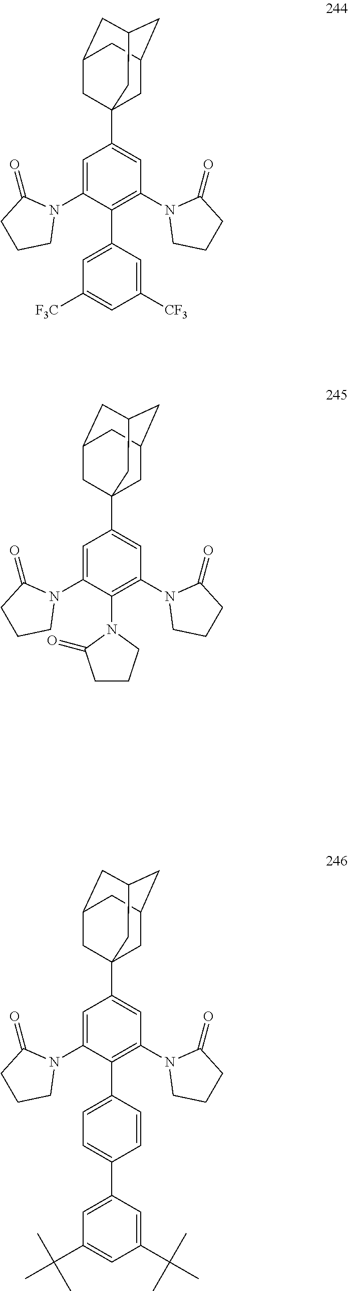 [SMILES: O=C1CCCN1c1cc(C23CC4CC(CC(C4)C2)C3)cc(N2CCCC2=O)c1-c1cc(C(F)(F)F)cc(C(F)(F)F)c1 ; O=C1CCCN1c1cc(C23CC4CC(CC(C4)C2)C3)cc(N2CCCC2=O)c1N1CCCC1=O ; CC(C)(C)c1cc(-c2ccc(-c3c(N4CCCC4=O)cc(C45CC6CC(CC(C6)C4)C5)cc3N3CCCC3=O)cc2)cc(C(C)(C)C)c1]

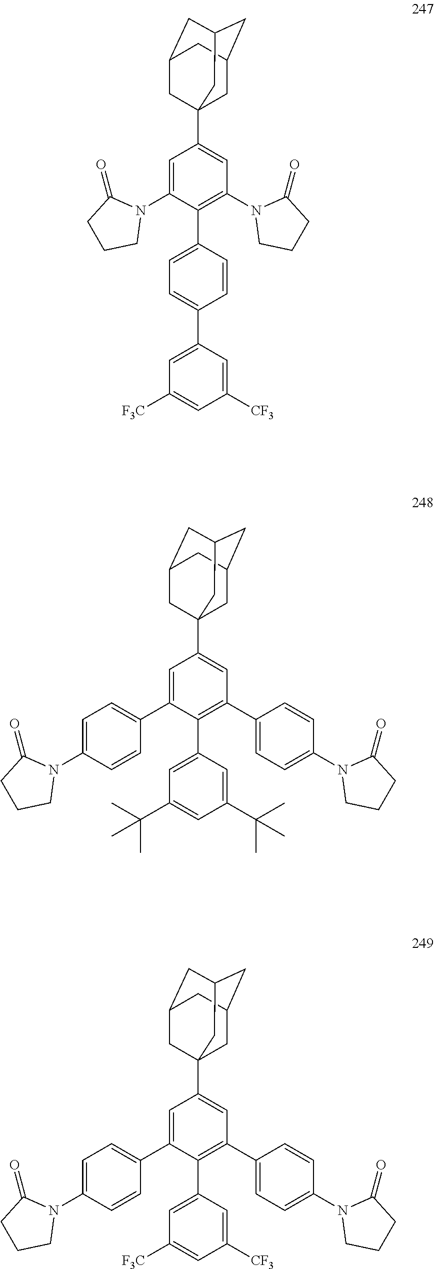 [SMILES: O=C1CCCN1c1cc(C23CC4CC(CC(C4)C2)C3)cc(N2CCCC2=O)c1-c1ccc(-c2cc(C(F)(F)F)cc(C(F)(F)F)c2)cc1 ; CC(C)(C)c1cc(-c2c(-c3ccc(N4CCCC4=O)cc3)cc(C34CC5CC(CC(C5)C3)C4)cc2-c2ccc(N3CCCC3=O)cc2)cc(C(C)(C)C)c1 ; O=C1CCCN1c1ccc(-c2cc(C34CC5CC(CC(C5)C3)C4)cc(-c3ccc(N4CCCC4=O)cc3)c2-c2cc(C(F)(F)F)cc(C(F)(F)F)c2)cc1]

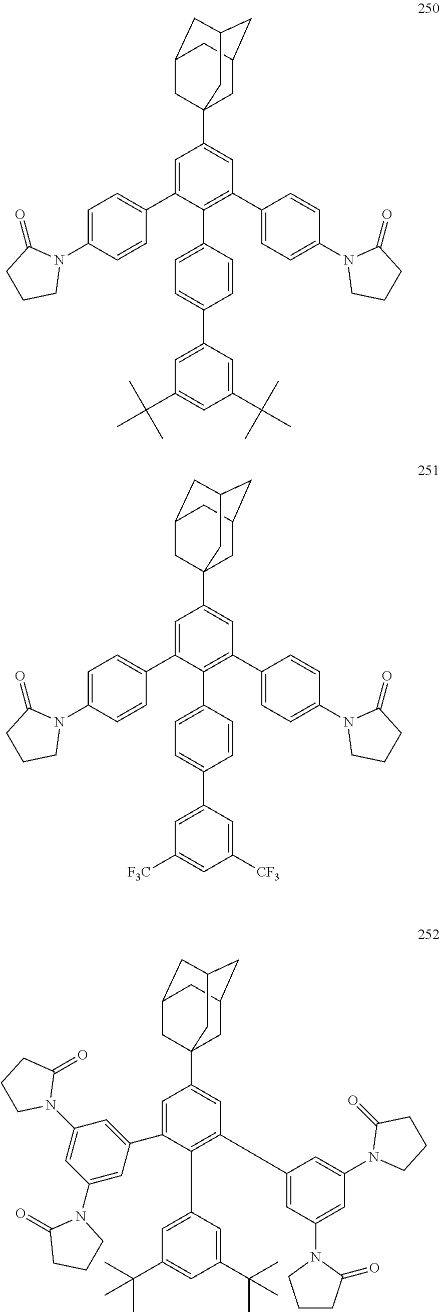

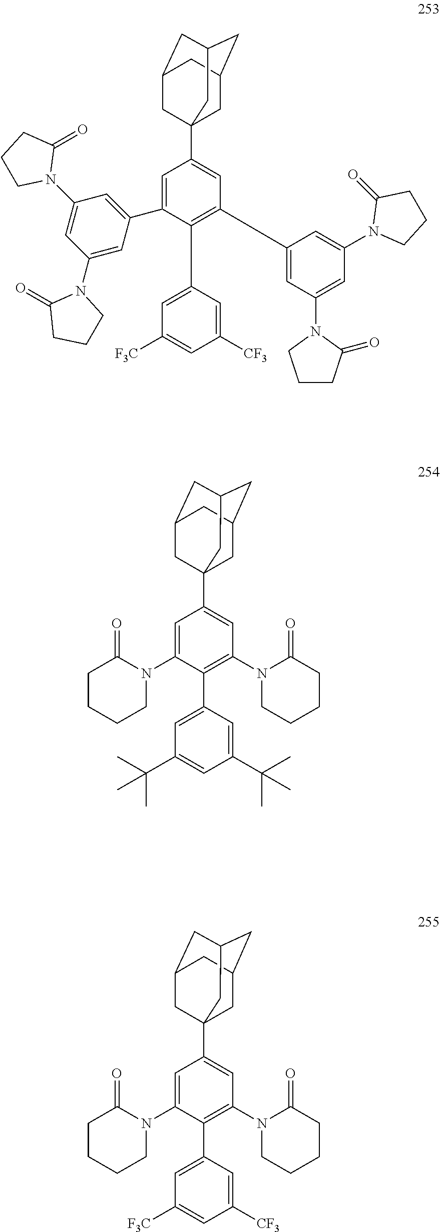 [SMILES: O=C1CCCN1c1cc(-c2cc(C34CC5CC(CC(C5)C3)C4)cc(-c3cc(N4CCCC4=O)cc(N4CCCC4=O)c3)c2-c2cc(C(F)(F)F)cc(C(F)(F)F)c2)cc(N2CCCC2=O)c1 ; CC(C)(C)c1cc(-c2c(N3CCCCC3=O)cc(C34CC5CC(CC(C5)C3)C4)cc2N2CCCCC2=O)cc(C(C)(C)C)c1 ; O=C1CCCCN1c1cc(C23CC4CC(CC(C4)C2)C3)cc(N2CCCCC2=O)c1-c1cc(C(F)(F)F)cc(C(F)(F)F)c1]

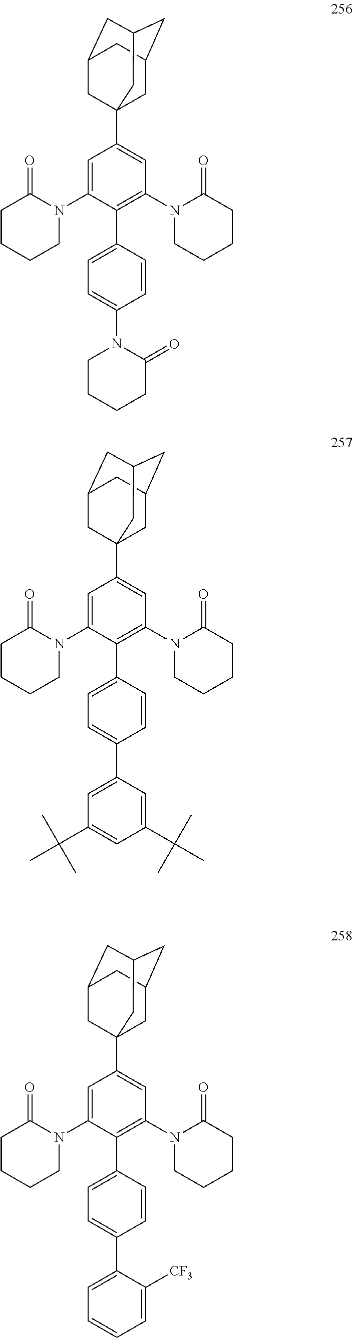

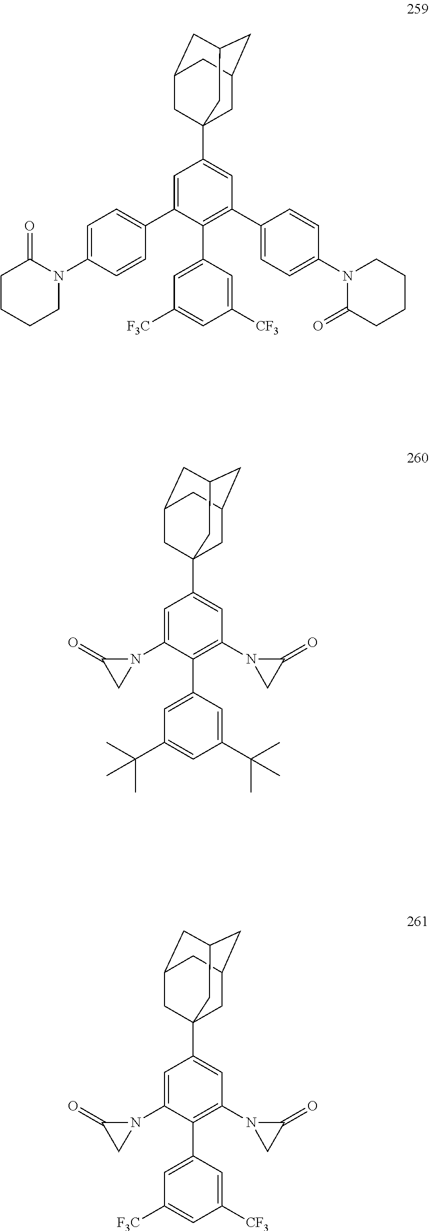 [SMILES: O=C1CCCCN1c1ccc(-c2cc(C34CC5CC(CC(C5)C3)C4)cc(-c3ccc(N4CCCCC4=O)cc3)c2-c2cc(C(F)(F)F)cc(C(F)(F)F)c2)cc1 ; CC(C)(C)c1cc(-c2c(N3CC3=O)cc(C34CC5CC(CC(C5)C3)C4)cc2N2CC2=O)cc(C(C)(C)C)c1 ; O=C1CN1c1cc(C23CC4CC(CC(C4)C2)C3)cc(N2CC2=O)c1-c1cc(C(F)(F)F)cc(C(F)(F)F)c1]

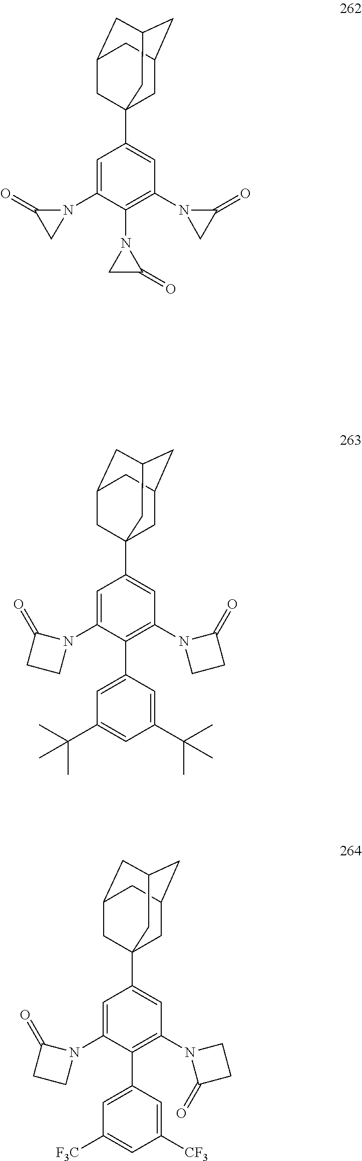 [SMILES: O=C1CN1c1cc(C23CC4CC(CC(C4)C2)C3)cc(N2CC2=O)c1N1CC1=O ; CC(C)(C)c1cc(-c2c(N3CCC3=O)cc(C34CC5CC(CC(C5)C3)C4)cc2N2CCC2=O)cc(C(C)(C)C)c1 ; O=C1CCN1c1cc(C23CC4CC(CC(C4)C2)C3)cc(N2CCC2=O)c1-c1cc(C(F)(F)F)cc(C(F)(F)F)c1]

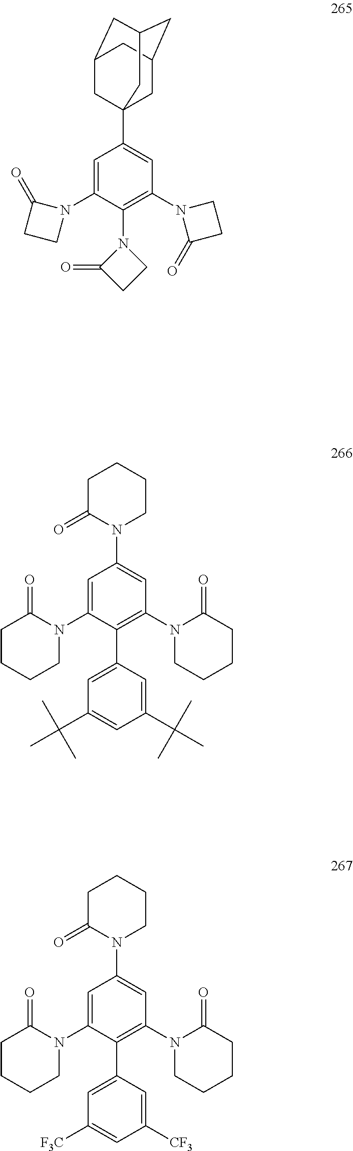 [SMILES: O=C1CCN1c1cc(C23CC4CC(CC(C4)C2)C3)cc(N2CCC2=O)c1N1CCC1=O ; CC(C)(C)c1cc(-c2c(N3CCCCC3=O)cc(N3CCCCC3=O)cc2N2CCCCC2=O)cc(C(C)(C)C)c1 ; O=C1CCCCN1c1cc(N2CCCCC2=O)c(-c2cc(C(F)(F)F)cc(C(F)(F)F)c2)c(N2CCCCC2=O)c1]

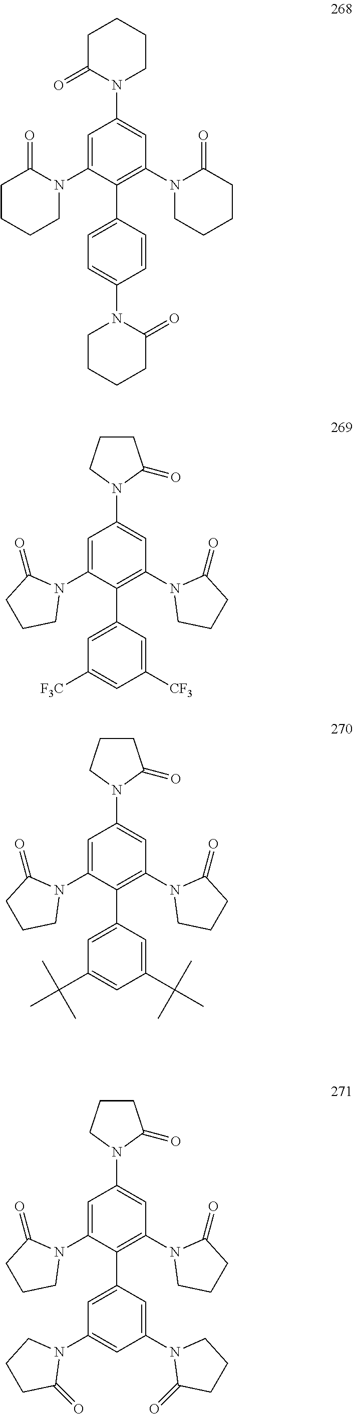 [SMILES: O=C1CCCCN1c1ccc(-c2c(N3CCCCC3=O)cc(N3CCCCC3=O)cc2N2CCCCC2=O)cc1 ; O=C1CCCN1c1cc(N2CCCC2=O)c(-c2cc(C(F)(F)F)cc(C(F)(F)F)c2)c(N2CCCC2=O)c1 ; CC(C)(C)c1cc(-c2c(N3CCCC3=O)cc(N3CCCC3=O)cc2N2CCCC2=O)cc(C(C)(C)C)c1 ; O=C1CCCN1c1cc(-c2c(N3CCCC3=O)cc(N3CCCC3=O)cc2N2CCCC2=O)cc(N2CCCC2=O)c1]

As described above, the organic compound according to the present invention may be implemented by synthesizing organic compounds having various properties using moieties having characteristic skeleton structures and inherent properties, and as a result, when the organic compound according to the present invention is applied to a light efficiency improving layer provided in an organic light-emitting device, it is possible to further improve not only the low voltage driving characteristics of the device, but also the light-emitting efficiency, color purity, service life characteristics, and the like.

In addition, the compound of the present invention may be applied to the device according to a general method for manufacturing an organic light emitting device. In addition, the compound of an embodiment of the present invention may be applied to a device according to a general method for manufacturing an organic light emitting device.

The organic light emitting device according to an exemplary embodiment of the present invention may be composed of a structure including a first electrode, a second electrode and an organic layer disposed therebetween, and may be manufactured using typical device manufacturing methods and materials, except that the organic light emitting compound according to the present invention is used in an organic layer of the device.

An organic light emitting device according to an embodiment of the present invention may include a first electrode, a second electrode, and an organic layer arranged therebetween. The organic light emitting device may be manufactured using a general device manufacturing method and material, except that the organic compound of an embodiment of the present invention is used to form the organic layer of the device.

The organic layer of the organic light emitting device according to the present invention may be composed of a single-layered structure, but may also be composed of a multi-layered structure in which two or more organic layers are stacked. For example, the organic layer may have a structure including a hole injection layer, a hole transport layer, a light-emitting layer, an electron transport layer, an electron injection layer, an electron blocking layer, a hole blocking layer, a light efficiency improving layer (capping layer), etc. However, the structure of the organic layer is not limited thereto, and may include a fewer or greater number of organic layers.

The organic layer of the organic light emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

In addition, the organic electroluminescent device may include a substrate, a first electrode (anode), an organic layer, a second electrode (cathode), and a light efficiency improving layer (capping layer), of which may be formed under the first electrode (bottom emission type) or on the second electrode (top emission type).

When the organic electroluminescent device is of a top emission type, light from the light emitting layer is emitted to the cathode and passes through the light efficiency improving layer (CPL) formed using the compound according to an embodiment of the present invention having a relatively high refractive index. The wavelength of the light is amplified, resulting in an increase in luminous efficiency.

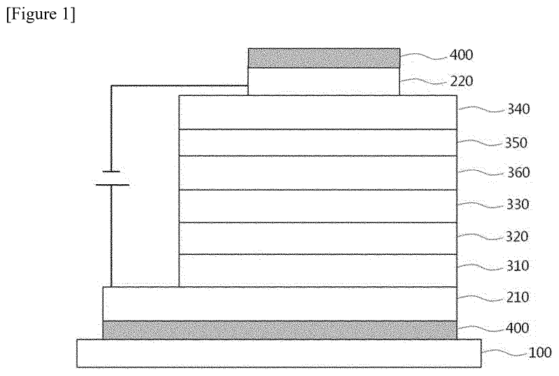

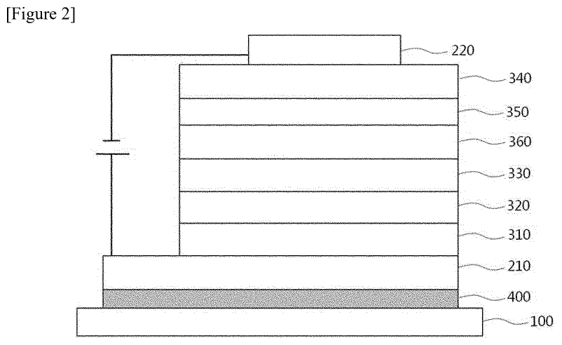

A specific embodiment of the organic light-emitting device according to the present invention will be described with reference to the following FIGS. 1 to 3.

The organic light-emitting device may include a substrate 100, a first electrode (210), a second electrode (220), one or more organic layers (310 to 360) interposed on the inner side of the first electrode and the second electrode, and a light efficiency improving layer (400), and the light efficiency improving layer may be disposed on the outer side of at least one of the first electrode and the second electrode.

Of both sides of the first electrode or the second electrode, the side adjacent to the organic layer interposed between the first electrode and the second electrode is referred to as the inner side, and the side not adjacent to the organic layer is referred to as the outer side. That is, accordingly, in the organic light-emitting device according to the present invention, when a light efficiency improving layer (400) is disposed on the outer side of the first electrode (210), the first electrode (210) is interposed between the light efficiency improving layer (400) and the organic layers (310 to 360), and when the light efficiency improving layer 400 is disposed on the outer side of the second electrode (220), the second electrode (220) is interposed between the light efficiency improving layer (400) and the organic layers (310 to 360).

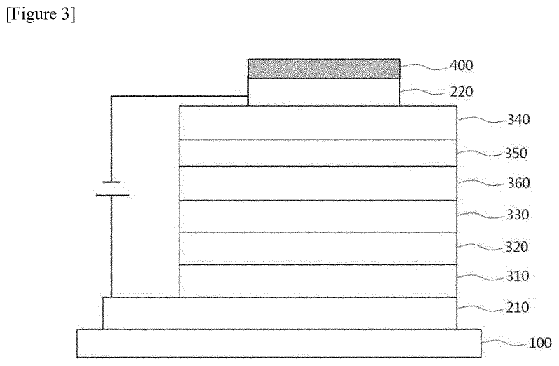

In this case, the method of forming a light efficiency improving layer on the second electrode as shown in the following FIG. 3 (top emission) may further include a reflection layer (not shown) to reflect light emitted between the first electrode (210) and the substrate (100), thereby further emitting the light toward the top of the second electrode.

As described above, for the organic light-emitting device according to the present invention, various organic layers having one or more layers may be interposed on the inner sides of the first electrode and the second electrode, and a light efficiency improving layer may be formed on the outer side of at least one of the first electrode and the second electrode. That is, the light efficiency improving layer may be formed on both the outer sides of the first electrode and the second electrode, or may be formed only on the outer side of the first electrode or only on the outer side of the second electrode.

In this case, the light efficiency improving layer may include a compound for a light efficiency improving layer according to the present invention, and may include a compound for a light efficiency improving layer according to the present invention alone or two or more of the compound or in combination with a known compound, and the light efficiency improving layer may have a thickness of 100 Å to 4,000 Å.

In addition, in the organic light-emitting device according to the present invention, the light efficiency improving layer may be configured as a composite light efficiency improving layer structure in which a first light efficiency improving layer having a relatively low refractive index and a second light efficiency improving layer having a higher refractive index than the first light efficiency improving layer are stacked, and the stacking order of the first light efficiency improving layer and the second light efficiency improving layer due to the difference in refractive index is not limited. The first light efficiency improving layer may be disposed on the outer side of the second light efficiency improving layer, and conversely, the second light efficiency improving layer may be disposed on the outer side of the first light efficiency improving layer.

Therefore, according to an embodiment of the present invention, the low refractive index compound represented by [Formula I] above according to the present invention may be applied to the first light efficiency improving layer.

Furthermore, each of the first light efficiency improving layer and the second light efficiency improving layer has a multi-layered structure in which a plurality of first light efficiency improving layers and a plurality of second light efficiency improving layers are stacked, and in this case, the first light efficiency improving layers and the second light efficiency improving layers may be stacked alternately, and the stacking order is also not limited.

Meanwhile, in the organic light-emitting device according to the present invention, the light efficiency improving layer may have a structure having a refractive index gradient, and for the refractive index gradient, the refractive index may gradually decrease toward the outside, or the refractive index may gradually increase toward the outside. For this purpose, the light efficiency improving layer may be deposited by gradually changing the concentration of the compound for the light efficiency improving layer according to the present invention, thereby implementing a gradient in the refractive index in the light efficiency improving layer.

Preferred structures of the organic layers of the organic light emitting according to an embodiment of the present invention will be explained in more detail in the examples to be described later.

Further, the organic light emitting device according to the present invention may be manufactured by depositing a metal or a metal oxide having conductivity, or an alloy thereof on a substrate to form a positive electrode, forming an organic layer including a hole injection layer, a hole transport layer, a light emitting layer, and an electron transport layer thereon, and then depositing a material, which may be used as a negative electrode, thereon, by using a physical vapor deposition (PVD) method such as sputtering or e-beam evaporation.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light emitting device may be fabricated by depositing a anode (210) material, organic layer materials (310˜360), and an cathode (220) material in this order on a substrate (100). The organic layers may have a multilayer structure including a hole injecting layer (310), a hole transport layer (320), an electron blocking layer (330), a light emitting layer (360), an electron transport layer (350) and an electron injecting layer (340), but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the substrate (100), a substrate commonly used in organic light-emitting devices may be used, and the substrate (100) may be particularly a transparent glass substrate or a flexible plastic substrate having excellent mechanical strength, thermal stability, transparency, surface smoothness, ease of handling, and water resistance.

As the anode (210) material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the cathode (220) material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting layer (310) material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport layer (320) material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting layer (360) material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly (p-phenylenevinylene) (PPV)-based polymers, spiro compounds, polyfluorene, and rubrene.

The electron transport layer (350) material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The electron injection layer (340) material may be formed by depositing an electron injection layer material on the electron transport layer, and a known material such as LiF, NaCl, CsF, Li₂O, and BaO may be used as the electron injection layer material.

Furthermore, although not shown in FIGS. 1 to 3, according to an embodiment of the present invention, organic layers having various functions may be additionally formed between the light efficiency improving layer (400) and the first electrode (210) or between the light efficiency improving layer (400) and the second electrode (220), and organic layers having various functions may also be additionally formed on the upper and lower parts (outer surfaces) of the capping layer (400).

The organic light emitting device according to the present disclosure may be a top-emission type, a bottom-emission type or a dual-emission type depending on the materials used.

The organic light emitting device according to an embodiment of the present invention may be of a top emission, bottom emission or dual emission type according to the materials used.

Furthermore, the organic light emitting compound according to the present invention may be operated by a principle which is similar to the principle applied to an organic light emitting device, even in an organic electroluminescent device including an organic solar cell, an organic photoconductor, an organic transistor, and the like.

In addition, the organic compound according to an embodiment of the present invention may perform its function even in organic electronic devices, including organic solar cells, organic photoconductors, and organic transistors, based on a similar principle to that applied to the organic light emitting device.

### Mode for Carrying Out Invention

Hereinafter, the present invention will be exemplified in more detail through preferred examples. However, these examples are for more specifically describing the present invention, the scope of the present invention is not limited thereto, and it will be obvious to a person with ordinary skill in the art that various changes and modifications can be made within the scope of the present invention and the scope of the technical spirit.

### Synthesis Example 1: Synthesis of Compound 3

### (1) Preparation Example 1: Synthesis of Intermediate 3-1

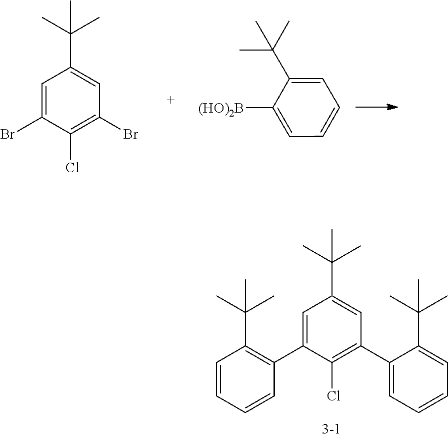 [SMILES: CC(C)(C)c1cc(Br)c(Cl)c(Br)c1 ; CC(C)(C)c1ccccc1B(O)O ; CC(C)(C)c1cc(-c2ccccc2C(C)(C)C)c(Cl)c(-c2ccccc2C(C)(C)C)c1]

200 mL of toluene, 50 mL of ethanol, and 50 mL of H₂O were added to 1,3-dibromo-2-chloro-5-(1,1-dimethylethyl)benzene (10.0 g, 0.031 mol), (2-tert-butylphenyl) boronic acid (13.1 g, 0.074 mol), K₂CO₃ (25.4 g, 0.184 mol), and Pd(PPh₃)₄ (0.7 g, 0.0006 mol), and the resulting mixture was stirred at 80° C. for 6 hours and reacted. After completion of the reaction, the resulting product was extracted and concentrated, and then subjected to column chromatography and recrystallization to obtain 8.5 g (yield 64.1%) of <Intermediate 3-1>.

### (2) Preparation Example 2: Synthesis of Compound 3

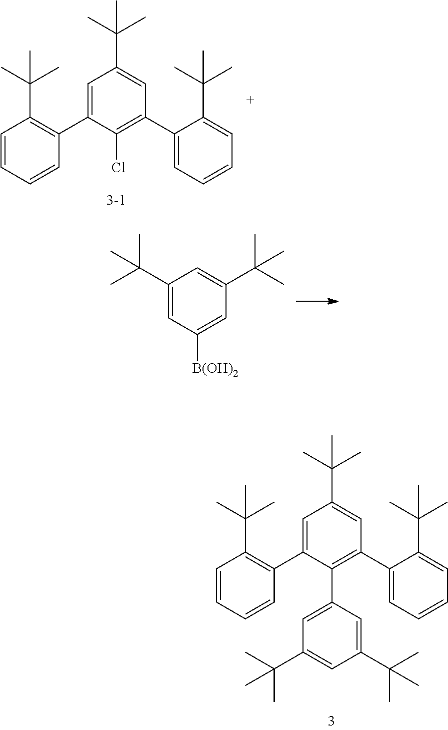

Intermediate 3-1 (10.0 g, 0.023 mol), (3,5-di-tert-butylphenyl) boronic acid (6.5 g, 0.028 mol), K₂CO₃ (9.6 g, 0.069 mol), Pd(OAc)₂ (1.3 g, 1.2 mmol), X-Phos (1.1 g, 2.3 mmol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 9.3 g (yield 68.6%) of <Compound 3>.

LC/MS: m/z=586 [(M)⁺]

### Synthesis Example 2: Synthesis of Compound 13

### (1) Preparation Example 1: Synthesis of Intermediate 13-1

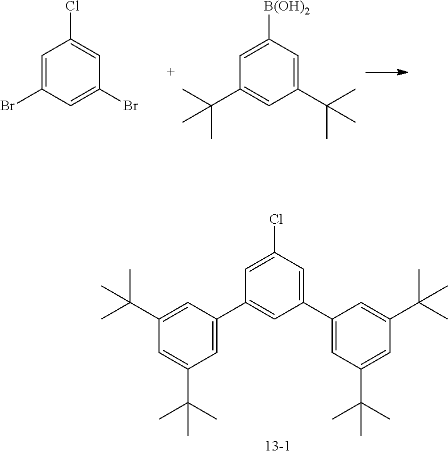 [SMILES: Clc1cc(Br)cc(Br)c1 ; CC(C)(C)c1cc(B(O)O)cc(C(C)(C)C)c1 ; CC(C)(C)c1cc(-c2cc(Cl)cc(-c3cc(C(C)(C)C)cc(C(C)(C)C)c3)c2)cc(C(C)(C)C)c1]

200 mL of toluene, 50 mL of ethanol, and 50 mL of H₂O were added to 1,3-dibromo-5-chlorobenzene (10.0 g, 0.037 mol), (3,5-di-tert-butylphenyl) boronic acid (20.8 g, 0.089 mol), K₂CO₃ (30.7 g, 0.222 mol), and Pd(PPh₃)₄ (0.9 g, 0.7 mmol), and the resulting mixture was stirred at 80° C. for 6 hours and reacted. After completion of the reaction, the resulting product was extracted and concentrated, and then subjected to column chromatography to obtain 13.8 g (yield 76.3%) of <Intermediate 13-1>.

### (2) Preparation Example 2: Synthesis of Intermediate 13-2

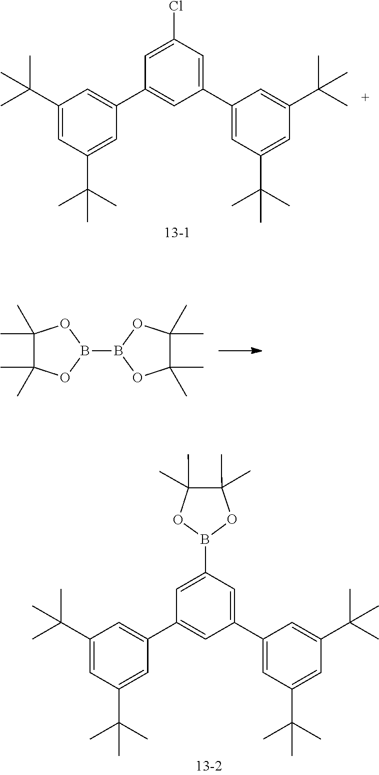

Dioxane was added to Intermediate 13-1 (10.0 g, 0.020 mol), bis(pinacolato)diboron (6.2 g, 0.025 mol), CH₃COOK (6.0 g, 0.061 mol), Pd(dppf)Cl₂ (0.8 g, 1.0 mmol), and XPhos (0.9 g, 1.8 mmol), and the resulting mixture was stirred at 100° C. for 12 hours and reacted. After completion of the reaction, the resulting product was extracted and concentrated, and then subjected to column chromatography and recrystallization to obtain 7.9 g (yield 66.6%) of <Intermediate 13-2>.

### (3) Preparation Example 3: Synthesis of Compound 13

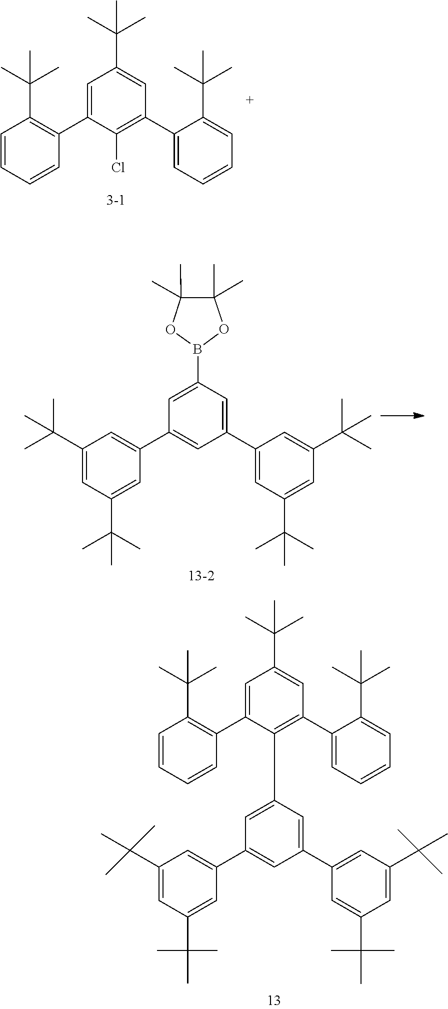

Intermediate 3-1 (10.0 g, 0.023 mol), Intermediate 13-2 (16.1 g, 0.028 mol), K₂CO₃ (9.6 g, 0.069 mol), Pd(OAc)₂ (1.3 g, 0.001 mol), X-Phos (2.2 g, 0.005 mol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 14.1 g (yield 71.7%) of <Compound 13>.

LC/MS: m/z=851 [(M)⁺]

### Synthesis Example 3: Synthesis of Compound 31

### (1) Preparation Example 1: Synthesis of Compound 31

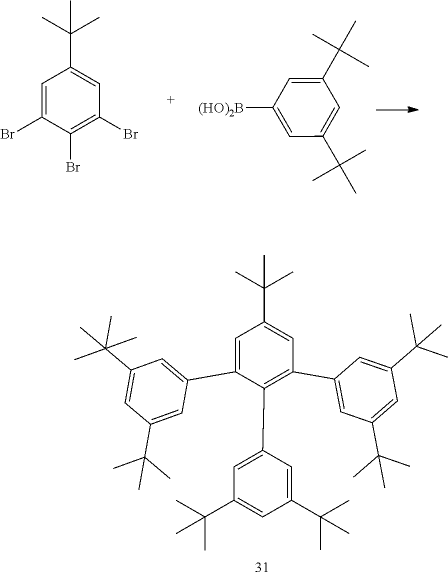

200 mL of toluene, 50 mL of ethanol, and 50 mL of H₂O were added to 1,2,3-tribromo-5-(1,1-dimethylethyl)benzene (10.0 g, 0.027 mol), (3,5-di-tert-butylphenyl) boronic acid (22.7 g, 0.097 mol), K₂CO₃ (33.5 g, 0.243 mol), and Pd(PPh₃)₄ (0.6 g, 0.0005 mol), and the resulting mixture was stirred at 80° C. for 6 hours and reacted. After completion of the reaction, the resulting product was extracted and concentrated, and then subjected to column chromatography and recrystallization to obtain 11.8 g (yield 62.6%) of <Compound 31>.

LC/MS: m/z=699 [(M)⁺]

### Synthesis Example 4: Synthesis of Compound 33

### (1) Preparation Example 1: Synthesis of Intermediate 33-1

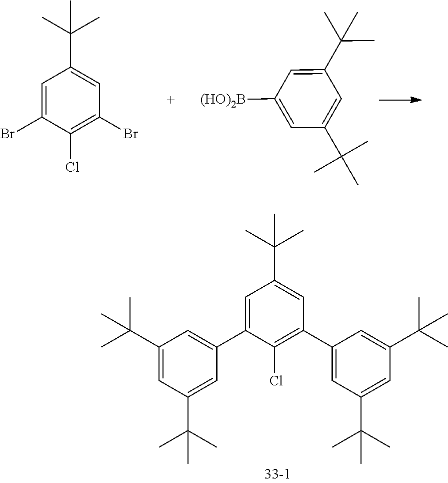 [SMILES: CC(C)(C)c1cc(Br)c(Cl)c(Br)c1 ; CC(C)(C)c1cc(B(O)O)cc(C(C)(C)C)c1 ; CC(C)(C)c1cc(-c2cc(C(C)(C)C)cc(-c3cc(C(C)(C)C)cc(C(C)(C)C)c3)c2Cl)cc(C(C)(C)C)c1]

200 mL of toluene, 50 mL of ethanol, and 50 mL of H₂O were added to 1,3-dibromo-2-chloro-5-(1,1-dimethylethyl)benzene (10.0 g, 0.033 mol), (3,5-di-tert-butylphenyl) boronic acid (18.4 g, 0.079 mol), K₂CO₃ (27.2 g, 0.197 mol), and Pd(PPh₃)₄ (0.8 g, 0.0007 mol), and the resulting mixture was stirred at 80° C. for 6 hours and reacted. After completion of the reaction, the resulting product was extracted and concentrated, and then subjected to column chromatography and recrystallization to obtain 12.2 g (yield 71.0%) of <Intermediate 33-1>.

### (2) Preparation Example 2: Synthesis of Compound 33

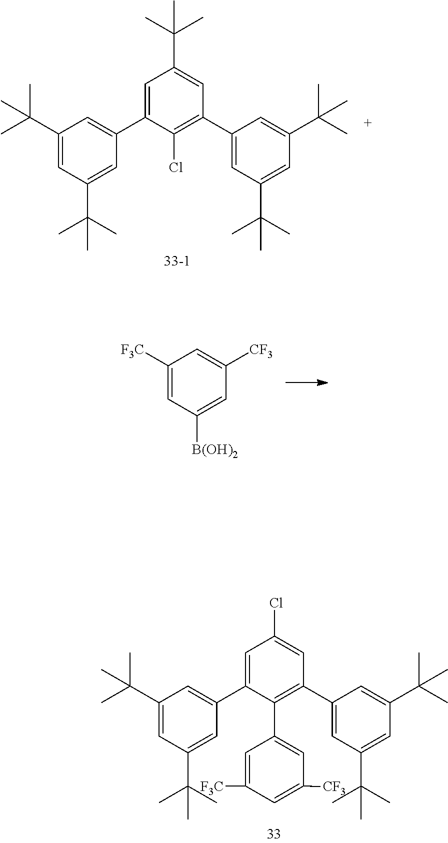

Intermediate 33-1 (10.0 g, 0.018 mol), 3,5-bis(trifluoromethyl)phenylboronic acid (5.7 g, 0.022 mol), K₂CO₃ (7.6 g, 0.055 mol), Pd(OAc)₂ (1.1 g, 0.001 mol), X-Phos (1.7 g, 0.004 mol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 7.5 g (yield 56.6%) of <Compound 33>.

LC/MS: m/z=722 [(M)⁺]

### Synthesis Example 5: Synthesis of Compound 67

### (1) Preparation Example 1: Synthesis of Compound 67

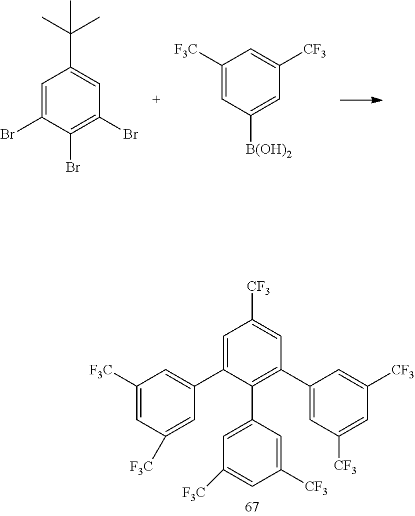 [SMILES: CC(C)(C)c1cc(Br)c(Br)c(Br)c1 ; OB(O)c1cc(C(F)(F)F)cc(C(F)(F)F)c1 ; FC(F)(F)c1cc(-c2cc(C(F)(F)F)cc(-c3cc(C(F)(F)F)cc(C(F)(F)F)c3)c2-c2cc(C(F)(F)F)cc(C(F)(F)F)c2)cc(C(F)(F)F)c1]

200 mL of toluene, 50 mL of ethanol, and 50 mL of H₂O were added to 1,2,3-tribromo-5-(1,1-dimethylethyl)benzene (10.0 g, 0.027 mol), 3,5-bis(trifluoromethyl)phenylboronic acid (25.0 g, 0.097 mol), K₂CO₃ (33.5 g, 0.243 mol), and Pd(PPh₃)₄ (0.6 g, 0.0005 mol), and the resulting mixture was stirred at 80° C. for 6 hours and reacted. After completion of the reaction, the resulting product was extracted and concentrated, and then subjected to column chromatography and recrystallization to obtain 11.2 g (yield 53.9%) of <Compound 67>.

LC/MS: m/z=770 [(M)⁺]

### Synthesis Example 6: Synthesis of Compound 77

### (1) Preparation Example 1: Synthesis of Intermediate 77-1

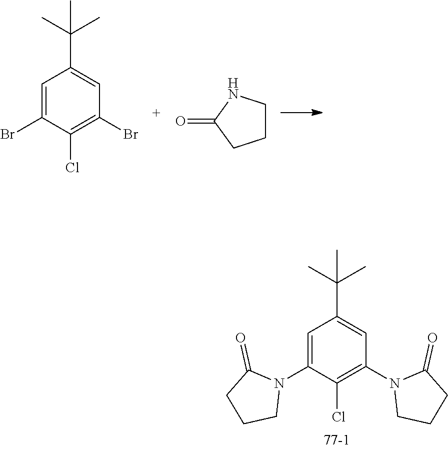 [SMILES: CC(C)(C)c1cc(Br)c(Cl)c(Br)c1 ; O=C1CCCN1 ; CC(C)(C)c1cc(N2CCCC2=O)c(Cl)c(N2CCCC2=O)c1]

1,3-dibromo-2-chloro-5-(1,1-dimethylethyl)benzene (10.0 g, 0.031 mol), 2-pyrrolidinone (6.3 g, 0.074 mol), K₃PO₄ (39.1 g, 0.184 mol), Pd(dba)₂ (1.8 g, 0.003 mol), Xant-Phos (12.8 g, 0.022 mol), and dioxane were put into a container, and the resulting mixture was stirred under reflux for 16 hours and reacted. After completion of the reaction, the resulting product was extracted and concentrated, and then subjected to column chromatography and recrystallization to obtain 8.5 g (yield 82.9%) of <Intermediate 77-1>.

### (2) Preparation Example 2: Synthesis of Compound 77

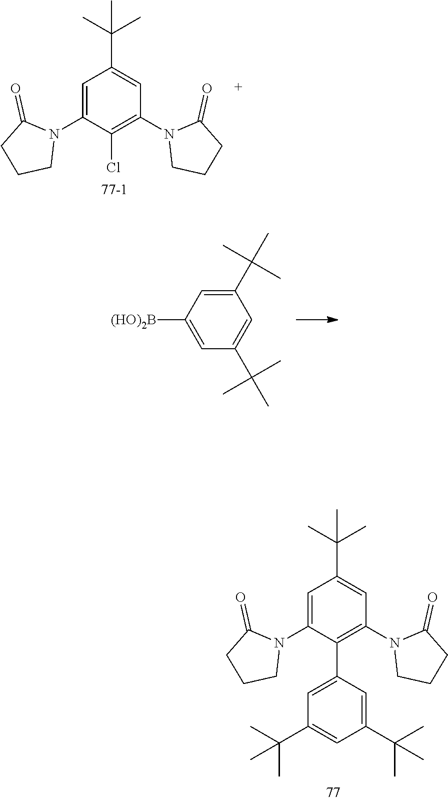 [SMILES: CC(C)(C)c1cc(N2CCCC2=O)c(Cl)c(N2CCCC2=O)c1 ; CC(C)(C)c1cc(B(O)O)cc(C(C)(C)C)c1 ; CC(C)(C)c1cc(-c2c(N3CCCC3=O)cc(C(C)(C)C)cc2N2CCCC2=O)cc(C(C)(C)C)c1]

Intermediate 77-1 (10.0 g, 0.030 mol), (3,5-di-tert-butylphenyl) boronic acid (8.4 g, 0.036 mol), K₂CO₃ (12.4 g, 0.090 mol), Pd(OAc)₂ (1.7 g, 0.002 mol), X-Phos (2.8 g, 0.006 mol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 9.1 g (yield 62.4%) of <Compound 77>.

LC/MS: m/z=488 [(M)⁺]

### Synthesis Example 7: Synthesis of Compound 78

### (1) Preparation Example 1: Synthesis of Compound 78

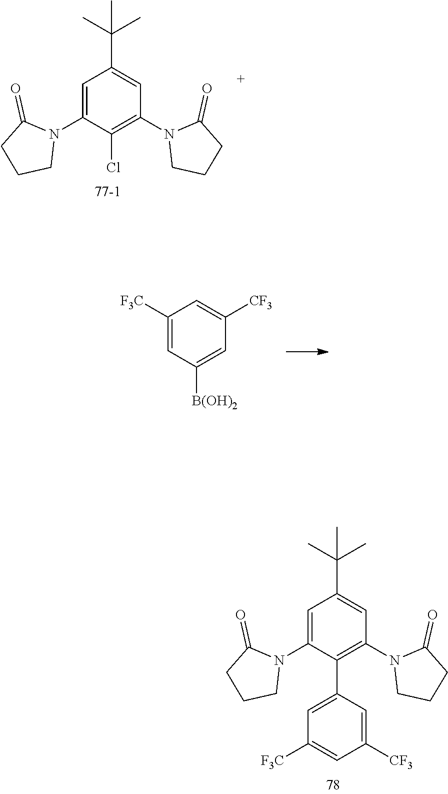 [SMILES: CC(C)(C)c1cc(N2CCCC2=O)c(Cl)c(N2CCCC2=O)c1 ; OB(O)c1cc(C(F)(F)F)cc(C(F)(F)F)c1 ; CC(C)(C)c1cc(N2CCCC2=O)c(-c2cc(C(F)(F)F)cc(C(F)(F)F)c2)c(N2CCCC2=O)c1]

Intermediate 77-1 (10.0 g, 0.030 mol), 3,5-bis(trifluoromethyl)phenylboronic acid (9.2 g, 0.036 mol), K₂CO₃ (12.4 g, 0.090 mol), Pd(OAc)₂ (1.7 g, 0.002 mol), X-Phos (2.8 g, 0.006 mol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 9.1 g (yield 59.5%) of <Compound 78>.

LC/MS: m/z=512 [(M)⁺]

### Synthesis Example 8: Synthesis of Compound 79

### (1) Preparation Example 1: Synthesis of Compound 79

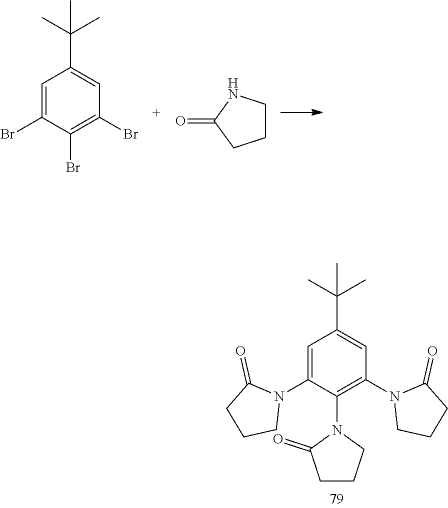 [SMILES: CC(C)(C)c1cc(Br)c(Br)c(Br)c1 ; O=C1CCCN1 ; CC(C)(C)c1cc(N2CCCC2=O)c(N2CCCC2=O)c(N2CCCC2=O)c1]

1,2,3-tribromo-5-(1,1-dimethylethyl)benzene (10.0 g, 0.027 mol), 2-pyrrolidinone (8.3 g, 0.097 mol), K₃PO₄ (51.5 g, 0.243 mol), Pd(dba)₂ (2.3 g, 0.004 mol), Xant-Phos (25.3 g, 0.044 mol), and dioxane were put into a container, and the resulting mixture was stirred under reflux for 16 hours and reacted. After completion of the reaction, the resulting product was extracted and concentrated, and then subjected to column chromatography and recrystallization to obtain 9.1 g (yield 87.1%) of <Compound 79>.

LC/MS: m/z=383 [(M)⁺]

### Synthesis Example 9: Synthesis of Compound 102

### (1) Preparation Example 1: Synthesis of Intermediate 102-1

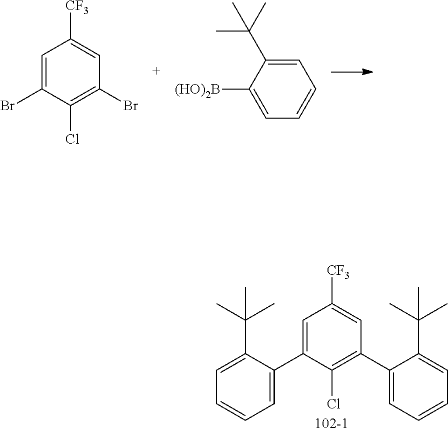 [SMILES: FC(F)(F)c1cc(Br)c(Cl)c(Br)c1 ; CC(C)(C)c1ccccc1B(O)O ; CC(C)(C)c1ccccc1-c1cc(C(F)(F)F)cc(-c2ccccc2C(C)(C)C)c1Cl]

200 mL of toluene, 50 mL of ethanol, and 50 mL of H₂O were added to 1,3-dibromo-2-chloro-5-(trifluoromethyl)benzene (10.0 g, 0.030 mol), (2-(tert-butyl)phenyl) boronic acid (12.6 g, 0.071 mol), K₂CO₃ (24.5 g, 0.178 mol), and Pd(PPh₃)₄ (0.7 g, 0.6 mmol), and the resulting mixture was stirred at 80° C. for 6 hours and reacted. After completion of the reaction, the resulting product was extracted and concentrated, and then subjected to column chromatography and recrystallization to obtain 8.6 g (yield 65.3%) of <Intermediate 102-1>.

### (2) Preparation Example 2: Synthesis of Compound 102

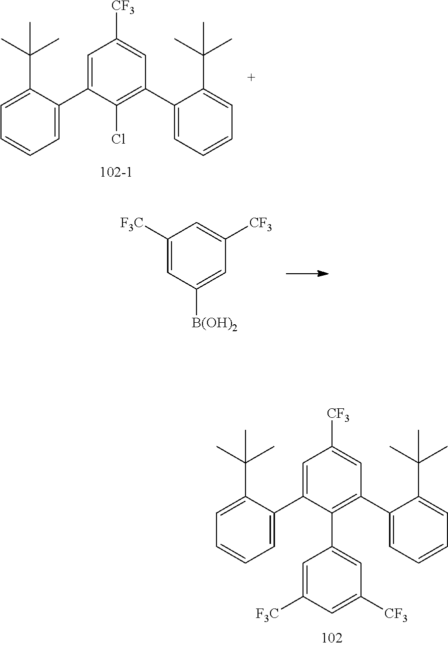 [SMILES: CC(C)(C)c1ccccc1-c1cc(C(F)(F)F)cc(-c2ccccc2C(C)(C)C)c1Cl ; OB(O)c1cc(C(F)(F)F)cc(C(F)(F)F)c1 ; CC(C)(C)c1ccccc1-c1cc(C(F)(F)F)cc(-c2ccccc2C(C)(C)C)c1-c1cc(C(F)(F)F)cc(C(F)(F)F)c1]

Intermediate 102-1 (10.0 g, 0.023 mol), (3,5-bis(trifluoromethyl)phenyl) boronic acid (7.0 g, 0.027 mol), K₂CO₃ (9.3 g, 0.067 mol), Pd(OAc)₂ (1.3 g, 1.1 mmol), X-Phos (1.1 g, 2.2 mmol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 8.5 g (yield 60.7%) of <Compound 102>.

LC/MS: m/z=622 [(M)⁺]

### Synthesis Example 10: Synthesis of Compound 118

### (1) Preparation Example 1: Synthesis of Intermediate 118-1

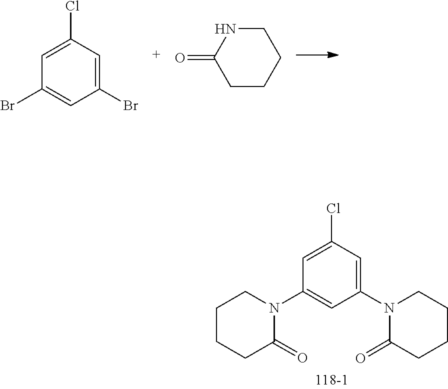 [SMILES: Clc1cc(Br)cc(Br)c1 ; O=C1CCCCN1 ; O=C1CCCCN1c1cc(Cl)cc(N2CCCCC2=O)c1]

1,3-dibromo-5-chlorobenzene (10.0 g, 0.037 mol), 2-piperidone (8.8 g, 0.089 mol), K₃PO₄ (47.2 g, 0.222 mol), Pd(dba)₂ (2.1 g, 3.7 mmol), Xant-Phos (15.4 g, 0.027 mol), and dioxane were put into a container, and the resulting mixture was stirred under reflux for 16 hours and reacted. After completion of the reaction, the resulting product was extracted and concentrated, and then subjected to column chromatography to obtain 9.3 g (yield 81.8%) of <Intermediate 118-1>.

### (2) Preparation Example 2: Synthesis of Intermediate 118-2

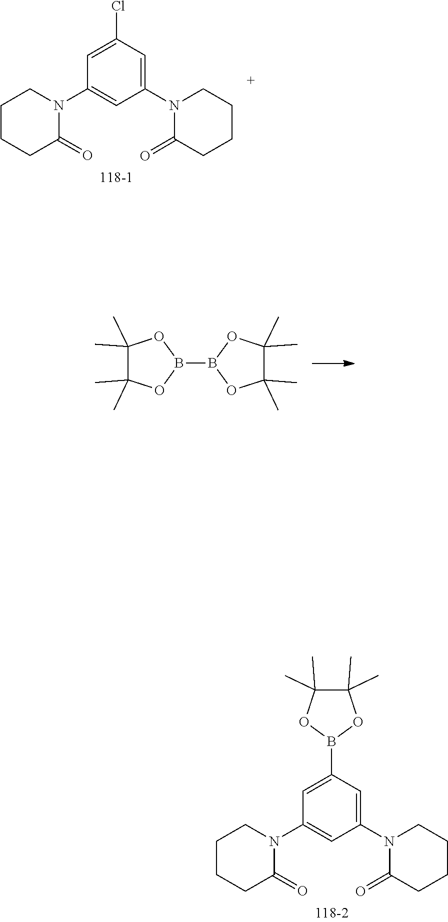

Dioxane was added to Intermediate 118-1 (10.0 g, 0.033 mol), bis(pinacolato)diboron (9.9 g, 0.039 mol), CH₃COOK (9.6 g, 0.098 mol), Pd(dppf)Cl₂ (1.2 g, 1.6 mmol), and XPhos (1.4 g, 2.9 mmol), and the resulting mixture was stirred at 100° C. for 12 hours and reacted. After completion of the reaction, the resulting product was extracted and concentrated, and then subjected to column chromatography and recrystallization to obtain 9.5 g (yield 73.3%) of <Intermediate 118-2>.

### (3) Preparation Example 3: Synthesis of Compound 118

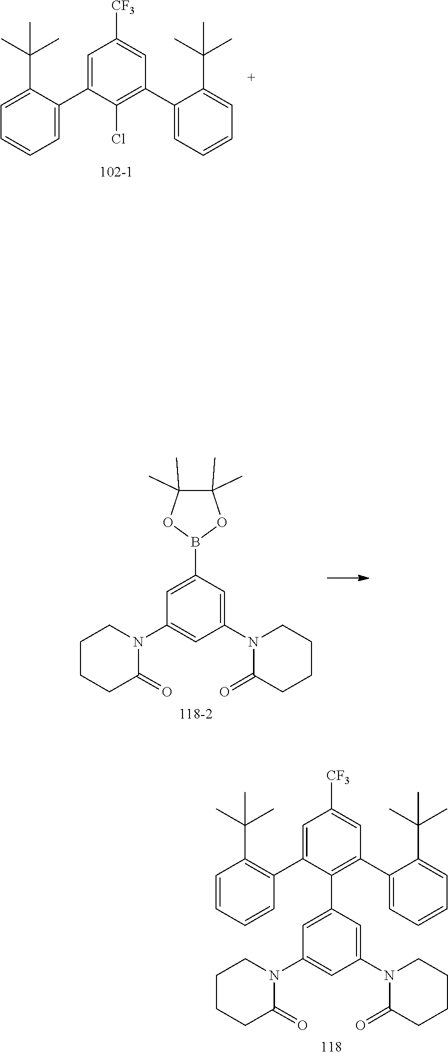 [SMILES: CC(C)(C)c1ccccc1-c1cc(C(F)(F)F)cc(-c2ccccc2C(C)(C)C)c1Cl ; CC1(C)OB(c2cc(N3CCCCC3=O)cc(N3CCCCC3=O)c2)OC1(C)C ; CC(C)(C)c1ccccc1-c1cc(C(F)(F)F)cc(-c2ccccc2C(C)(C)C)c1-c1cc(N2CCCCC2=O)cc(N2CCCCC2=O)c1]

Intermediate 102-1 (10.0 g, 0.023 mol), Intermediate 118-2 (10.7 g, 0.027 mol), K₂CO₃ (9.3 g, 0.067 mol), Pd(OAc)₂ (1.1 g, 1.1 mmol), X-Phos (1.1 g, 2.2 mmol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 9.2 g (yield 60.1%) of <Compound 118>.

### Synthesis Example 11: Synthesis of Compound 143

### (1) Preparation Example 1: Synthesis of Intermediate 143-1

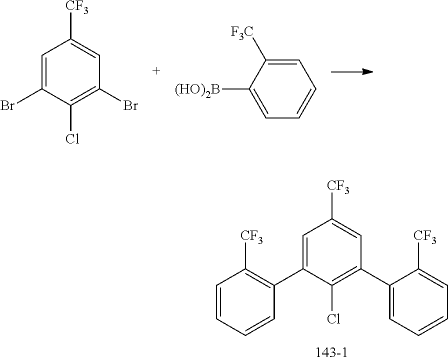 [SMILES: FC(F)(F)c1cc(Br)c(Cl)c(Br)c1 ; OB(O)c1ccccc1C(F)(F)F ; FC(F)(F)c1cc(-c2ccccc2C(F)(F)F)c(Cl)c(-c2ccccc2C(F)(F)F)c1]

200 mL of toluene, 50 mL of ethanol, and 50 mL of H₂O were added to 1,3-dibromo-2-chloro-5-(trifluoromethyl)benzene (10.0 g, 0.030 mol), 2-(trifluoromethyl)phenylboronic acid (13.5 g, 0.071 mol), K₂CO₃ (24.5 g, 0.177 mol), and Pd(PPh₃)₄ (0.7 g, 0.0006 mol), and the resulting mixture was stirred at 80° C. for 6 hours and reacted. After completion of the reaction, the resulting product was extracted and concentrated, and then subjected to column chromatography and recrystallization to obtain 8.9 g (yield 64.2%) of <Intermediate 143-1>.

### (2) Preparation Example 2: Synthesis of Compound 143

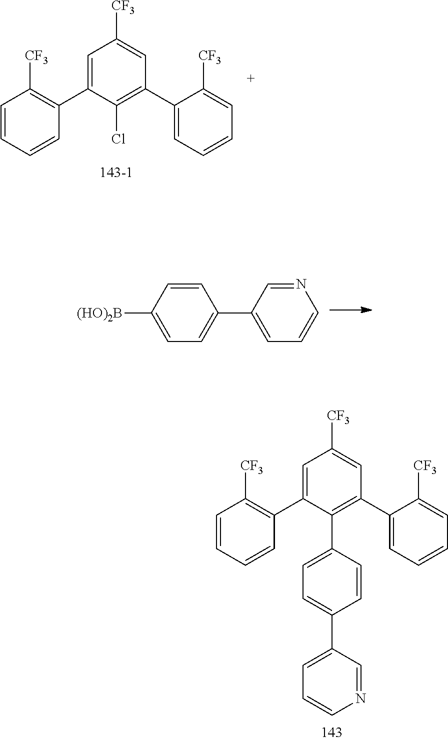 [SMILES: FC(F)(F)c1cc(-c2ccccc2C(F)(F)F)c(Cl)c(-c2ccccc2C(F)(F)F)c1 ; OB(O)c1ccc(-c2cccnc2)cc1 ; FC(F)(F)c1cc(-c2ccccc2C(F)(F)F)c(-c2ccc(-c3cccnc3)cc2)c(-c2ccccc2C(F)(F)F)c1]

Intermediate 143-1 (10.0 g, 0.021 mol), B-[4-(3-pyridinyl)phenyl]boronic acid (5.1 g, 0.026 mol), K₂CO₃ (8.9 g, 0.064 mol), Pd(OAc)₂ (1.2 g, 0.001 mol), X-Phos (2.0 g, 0.004 mol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 9.5 g (yield 75.8%) of <Compound 143>.

LC/MS: m/z=587 [(M)⁺]

### Synthesis Example 12: Synthesis of Compound 152

### (1) Preparation Example 1: Synthesis of Intermediate 152-1

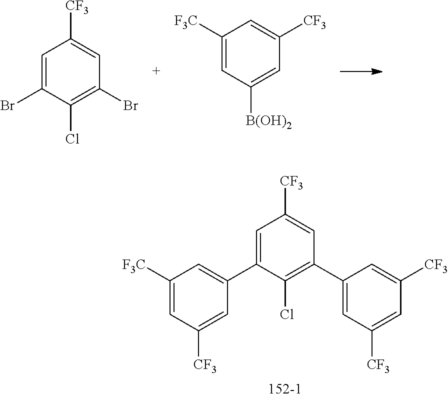 [SMILES: FC(F)(F)c1cc(Br)c(Cl)c(Br)c1 ; OB(O)c1cc(C(F)(F)F)cc(C(F)(F)F)c1 ; FC(F)(F)c1cc(-c2cc(C(F)(F)F)cc(-c3cc(C(F)(F)F)cc(C(F)(F)F)c3)c2Cl)cc(C(F)(F)F)c1]

200 mL of toluene, 50 mL of ethanol, and 50 mL of H₂O were added to 1,3-dibromo-2-chloro-5-(trifluoromethyl)benzene (10.0 g, 0.030 mol), (3,5-bis(trifluoromethyl)phenyl) boronic acid (18.3 g, 0.071 mol), K₂CO₃ (24.5 g, 0.178 mol), and Pd(PPh₃)₄ (0.7 g, 0.6 mmol), and the resulting mixture was stirred at 80° C. for 6 hours and reacted. After completion of the reaction, the resulting product was extracted and concentrated, and then subjected to column chromatography to obtain 10.3 g (yield 57.5%) of <Intermediate 152-1>.

### (2) Preparation Example 2: Synthesis of Intermediate 152-2

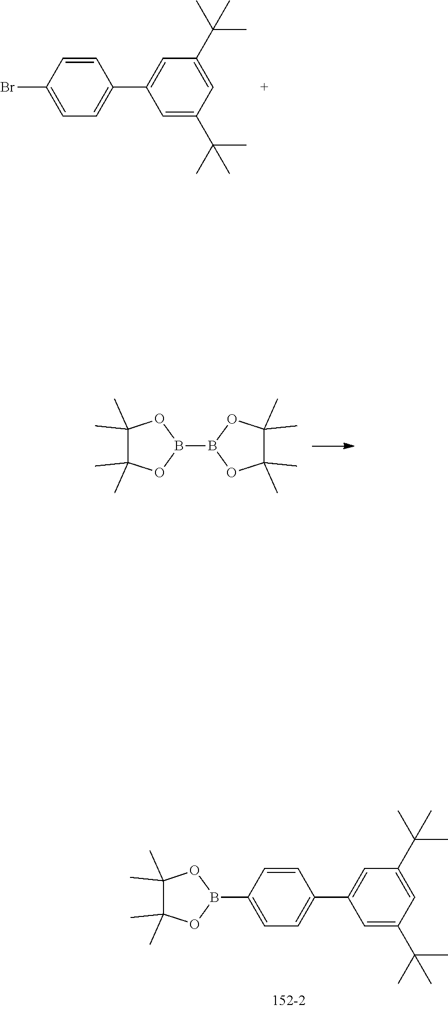

Dioxane was added to 4′-bromo-3,5-di-tert-butylbiphenyl (10.0 g, 0.029 mol), bis(pinacolato)diboron (8.8 g, 0.035 mol), CH₃COOK (8.5 g, 0.087 mol), and Pd(dppf)Cl₂ (1.1 g, 1.4 mmol), and the resulting mixture was stirred at 100° C. for 12 hours and reacted. After completion of the reaction, the resulting product was extracted and concentrated, and then subjected to column chromatography and recrystallization to obtain 8.6 g (yield 75.7%) of <Intermediate 152-2>.

### (3) Preparation Example 3: Synthesis of Compound 152

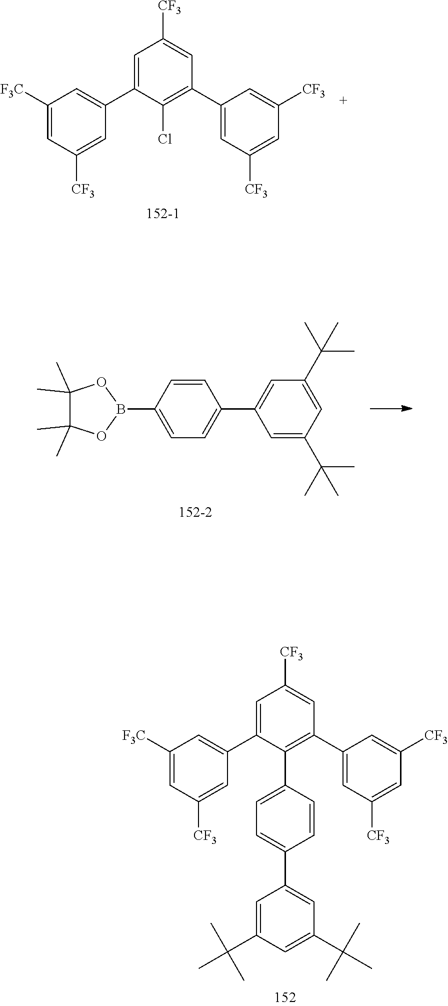 [SMILES: FC(F)(F)c1cc(-c2cc(C(F)(F)F)cc(-c3cc(C(F)(F)F)cc(C(F)(F)F)c3)c2Cl)cc(C(F)(F)F)c1 ; CC(C)(C)c1cc(-c2ccc(B3OC(C)(C)C(C)(C)O3)cc2)cc(C(C)(C)C)c1 ; CC(C)(C)c1cc(-c2ccc(-c3c(-c4cc(C(F)(F)F)cc(C(F)(F)F)c4)cc(C(F)(F)F)cc3-c3cc(C(F)(F)F)cc(C(F)(F)F)c3)cc2)cc(C(C)(C)C)c1]

Intermediate 152-1 (10.0 g, 0.017 mol), Intermediate 152-2 (7.8 g, 0.020 mol), K₂CO₃ (6.9 g, 0.050 mol), Pd(OAc)₂ (1.0 g, 0.8 mmol), X-Phos (0.8 g, 1.7 mmol), 200 mL of THF 200 mL, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 7.6 g (yield 55.1%) of <Compound 152>.

LC/MS: m/z=834 [(M)⁺]

### Synthesis Example 13: Synthesis of Compound 213

### (1) Preparation Example 1: Synthesis of Intermediate 213-1

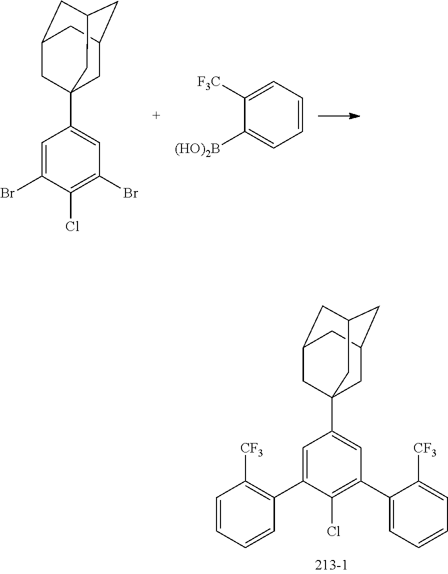 [SMILES: Clc1c(Br)cc(C23CC4CC(CC(C4)C2)C3)cc1Br ; OB(O)c1ccccc1C(F)(F)F ; FC(F)(F)c1ccccc1-c1cc(C23CC4CC(CC(C4)C2)C3)cc(-c2ccccc2C(F)(F)F)c1Cl]

200 mL of toluene, 50 mL of ethanol, and 50 mL of H₂O were added to 1-(3,5-dibromo-4-chlorophenyl) adamantane (10.0 g, 0.025 mol), (2-(trifluoromethyl)phenyl) boronic acid (11.3 g, 0.059 mol), K₂CO₃ (20.5 g, 0.148 mol), and Pd(PPh₃)₄ (0.6 g, 0.5 mmol), and the resulting mixture was stirred at 80° C. for 6 hours and reacted. After completion of the reaction, the resulting product was extracted and concentrated, and then subjected to column chromatography and recrystallization to obtain 6.2 g (yield 46.9%) of <Intermediate 213-1>.

### (2) Preparation Example 2: Synthesis of Compound 213

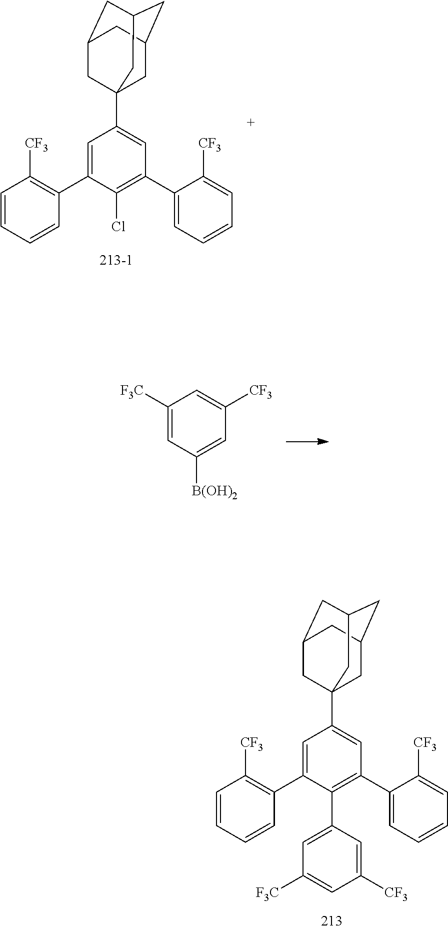 [SMILES: FC(F)(F)c1ccccc1-c1cc(C23CC4CC(CC(C4)C2)C3)cc(-c2ccccc2C(F)(F)F)c1Cl ; OB(O)c1cc(C(F)(F)F)cc(C(F)(F)F)c1 ; FC(F)(F)c1cc(-c2c(-c3ccccc3C(F)(F)F)cc(C34CC5CC(CC(C5)C3)C4)cc2-c2ccccc2C(F)(F)F)cc(C(F)(F)F)c1]

Intermediate 213-1 (10.0 g, 0.019 mol), (3,5-bis(trifluoromethyl)phenyl) boronic acid (5.8 g, 0.022 mol), K₂CO₃ (7.8 g, 0.056 mol), Pd(OAc)₂ (1.1 g, 0.9 mmol), X-Phos (0.9 g, 1.9 mmol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 6.8 g (yield 51.0%) of <Compound 213>.

LC/MS: m/z=712 [(M)⁺]

### Synthesis Example 14: Synthesis of Compound 243

### (1) Preparation Example 1: Synthesis of Intermediate 243-1

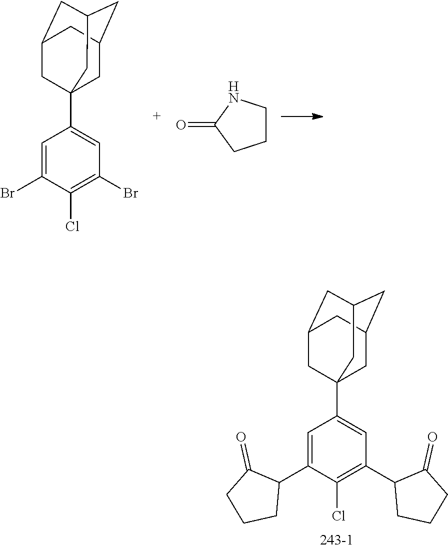 [SMILES: Clc1c(Br)cc(C23CC4CC(CC(C4)C2)C3)cc1Br ; O=C1CCCN1 ; O=C1CCCC1c1cc(C23CC4CC(CC(C4)C2)C3)cc(C2CCCC2=O)c1Cl]

1-(3,5-dibromo-4-chlorophenyl) adamantane (10.0 g, 0.025 mol), 2-pyrrolidinone (5.0 g, 0.059 mol), K₃PO₄ (31.5 g, 0.148 mol), Pd(dba)₂ (1.4 g, 2.5 mmol), Xant-Phos (10.3 g, 0.018 mol), and dioxane were put into a container, and the resulting mixture was stirred under reflux for 16 hours and reacted. After completion of the reaction, the resulting product was extracted and concentrated, and then subjected to column chromatography and recrystallization to obtain 7.8 g (yield 76.5%) of <Intermediate 243-1>.

### (2) Preparation Example 2: Synthesis of Compound 243

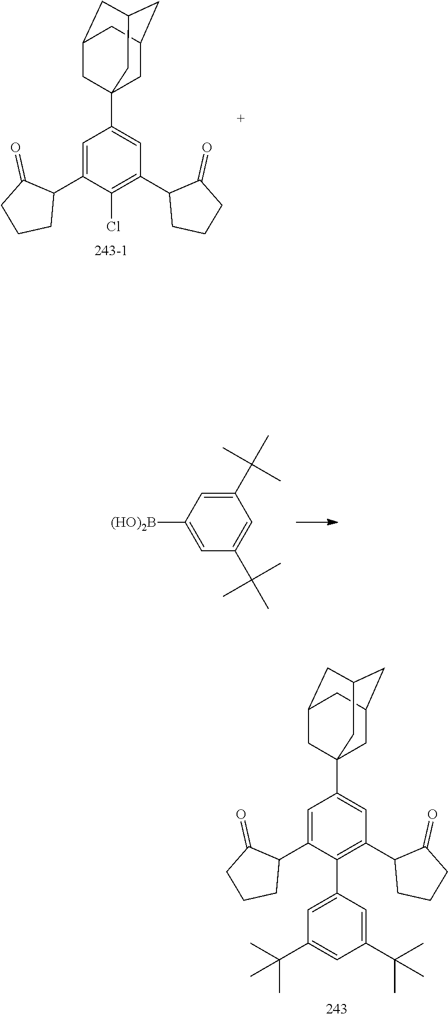 [SMILES: O=C1CCCC1c1cc(C23CC4CC(CC(C4)C2)C3)cc(C2CCCC2=O)c1Cl ; CC(C)(C)c1cc(B(O)O)cc(C(C)(C)C)c1 ; CC(C)(C)c1cc(-c2c(C3CCCC3=O)cc(C34CC5CC(CC(C5)C3)C4)cc2C2CCCC2=O)cc(C(C)(C)C)c1]

Intermediate 243-1 (10.0 g, 0.024 mol), (3,5-di-tert-butylphenyl) boronic acid (6.8 g, 0.029 mol), K₂CO₃ (10.0 g, 0.073 mol), Pd(OAc)₂ (1.4 g, 1.2 mmol), X-Phos (1.2 g, 2.4 mmol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 9.5 g (yield 75.8%) of <Compound 243>.

LC/MS: m/z=566 [(M)⁺]

### Synthesis Example 15: Synthesis of Compound 269

### (1) Preparation Example 1: Synthesis of Intermediate 269-1

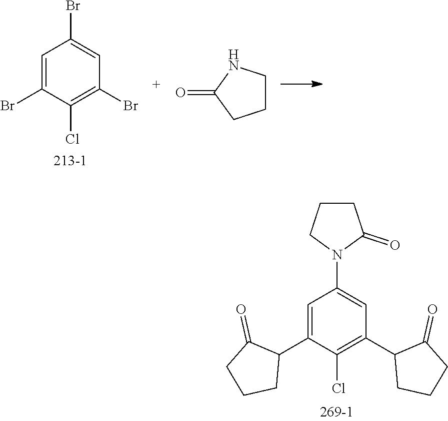 [SMILES: Clc1c(Br)cc(Br)cc1Br ; O=C1CCCN1 ; O=C1CCCC1c1cc(N2CCCC2=O)cc(C2CCCC2=O)c1Cl]

1,3,5-tribromo-2-chlorobenzene (10.0 g, 0.029 mol), 2-pyrrolidinone (8.8 g, 0.103 mol), K₃PO₄ (54.7 g, 0.258 mol), Pd(dba)₂ (2.5 g, 4.3 mmol), Xant-Phos (26.9 g, 0.046 mol), and dioxane were put into a container, and the resulting mixture was stirred under reflux for 16 hours and reacted. After completion of the reaction, the resulting product was extracted and concentrated, and then subjected to column chromatography and recrystallization to obtain 7.5 g (yield 72.3%) of <Intermediate 269-1>.

### (2) Preparation Example 2: Synthesis of Compound 269

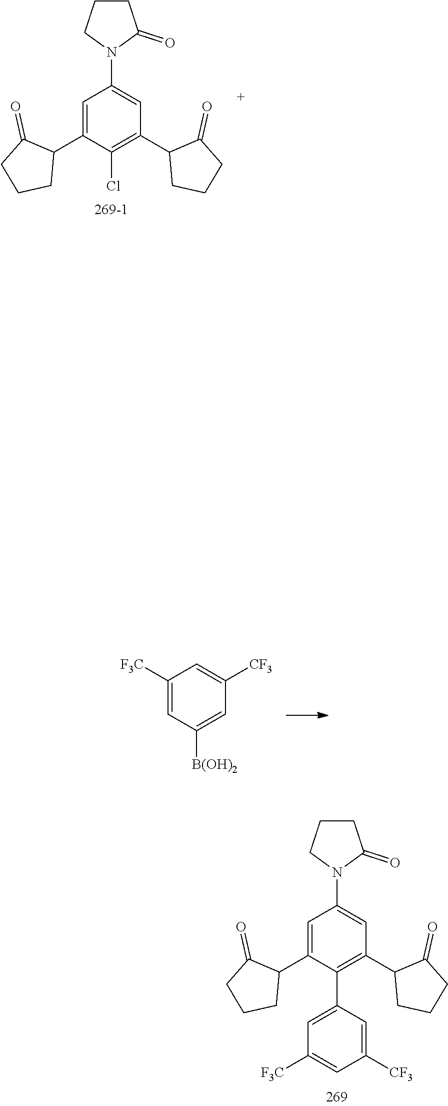 [SMILES: O=C1CCCC1c1cc(N2CCCC2=O)cc(C2CCCC2=O)c1Cl ; OB(O)c1cc(C(F)(F)F)cc(C(F)(F)F)c1 ; O=C1CCCC1c1cc(N2CCCC2=O)cc(C2CCCC2=O)c1-c1cc(C(F)(F)F)cc(C(F)(F)F)c1]

Intermediate 269-1 (10.0 g, 0.028 mol), (3,5-bis(trifluoromethyl)phenyl) boronic acid (8.6 g, 0.033 mol), K₂CO₃ (11.5 g, 0.083 mol), Pd(OAc)₂ (1.6 g, 1.4 mmol), X-Phos (1.3 g, 2.8 mmol), 200 mL of THF, and 50 mL of H₂O were put into a container, and the resulting mixture was stirred at 70° C. for 6 hours and reacted. After completion of the reaction, the resulting product was extracted and concentrated, and then subjected to column chromatography and recrystallization to obtain 6.2 g (yield 41.6%) of <Compound 269>.

LC/MS: m/z=539 [(M)⁺]

### Experimental Example 1: Optical Properties of Compound According to Present Invention

In an experimental example according to the present invention, a quartz glass having a size of 25 mm×25 mm was cleaned. Thereafter, when the base pressure reached 1×10⁻⁶ torr or more by placing the quartz glass in a vacuum chamber, the optical properties were measured by each depositing the compound according to the present invention and the comparative compound on a glass substrate.

### Device Examples 1 to 15

As a compound employed in the light efficiency improving layer provided in the organic light emitting device, each of the compounds according to the present invention shown in the following Table 1 was deposited on a glass substrate to a thickness of 100 nm to measure a refractive index.

Quartz glass/Organic material (100 nm)

### Comparative Example 1

Optical properties were measured by manufacturing the substrate for Comparative Example 1 in the same manner as in Examples 1 to 15, except that the following [CP1-1] was used instead of the compounds of Examples 1 to 15.

### Experimental Example 1: Optical Properties of Experimental Examples 1 to 15

The refractive indices of the substrates manufactured according to the Examples and Comparative Example were measured using ellipsometry (Elli-SE). The refractive index was measured in the blue wavelength region (450 nm), and the results are shown in the following Table 1.

TABLE 1

| Classification | Refractive index (450 nm) |
|---|---|
| Example 1 (Compound 3) | 1.70 |
| Example 2 (Compound 13) | 1.73 |
| Example 3 (Compound 31) | 1.64 |
| Example 4 (Compound 33) | 1.66 |
| Example 5 (Compound 67) | 1.75 |
| Example 6 (Compound 77) | 1.77 |
| Example 7 (Compound 78) | 1.63 |
| Example 8 (Compound 79) | 1.68 |
| Example 9 (Compound 102) | 1.75 |
| Example 10 (Compound 118) | 1.69 |
| Example 11 (Compound 143) | 1.72 |
| Example 12 (Compound 152) | 1.65 |
| Example 13 (Compound 213) | 1.68 |
| Example 14 (Compound 243) | 1.74 |
| Example 15 (Compound 269) | 1.71 |
| Comparative Example 1 (CP1-1) | 1.80 |

As can be seen from [Table 1] above, the refractive index value of the compound according to the present invention in a wavelength band of 450 nm is remarkably lower than that of the compound of Comparative Example 1, and when the compound according to the present invention having such a low refractive index value is employed in a light efficiency improving layer of an organic light-emitting device, it can be expected to optimize the efficiency of the device.

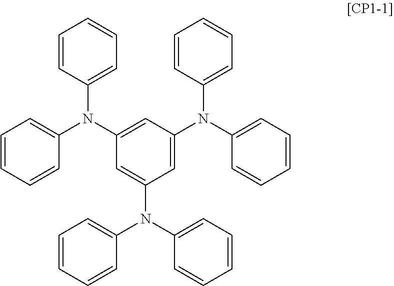 [SMILES: c1ccc(N(c2ccccc2)c2cc(N(c3ccccc3)c3ccccc3)cc(N(c3ccccc3)c3ccccc3)c2)cc1]

### Device Example (Second CPL/First CPL)

In embodiments according to the present invention, an anode was patterned using an ITO glass substrate including Ag of 25 mm×25 mm×0.7 mm such that a light emitting area had a size of 2 mm×2 mm, and then cleaned. After the patterned ITO substrate was mounted in a vacuum chamber, an organic material and a metal were deposited on the substrate at a process pressure of 1×10⁶ torr or more as the following structure.

### Device Examples 16 to 69

In Examples 16 to 69, blue organic light-emitting devices having the following device structure, particularly those composed of a plurality of light efficiency improving layers (second light efficiency improving layer/first light efficiency improving layer), were manufactured, and then the light emitting and driving characteristics thereof were measured.

Ag/ITO/hole injection layer (HAT-CN, 5 nm)/hole transport layer (HT1, 100 nm)/electron blocking layer (EB1, 10 nm)/light-emitting layer (20 nm)/electron transport layer (ET1:Liq, 30 nm)/LiF (1 nm)/Mg:Ag (15 nm)/second light efficiency improving layer (55 nm)/first light efficiency improving layer (10 nm).

After [HAT-CN] was film-formed to a thickness of 5 nm on an ITO transparent electrode including Ag on a glass substrate, [HT1] was film-formed to a thickness of 100 nm to form a hole transport layer, and then [EB1] was film-formed to a thickness of 10 nm to form an electron blocking layer. Thereafter, a light-emitting layer was formed by co-depositing [BH1] and [BD1] to a thickness of 20 nm using [BH1] as a host compound and [BD1] as a dopant compound. Thereafter, an electron transport layer (50% doping of the following [ET1] compound Liq) was deposited to a thickness of 30 nm, and then LiF was film-formed to a thickness of 1 nm to form an electron injection layer. Thereafter, Mg and Ag were film-formed at a ratio of 1:9 to a thickness of 15 nm to form a cathode.

And, the light efficiency improving layer (capping layer) was composed of the second light efficiency improving layer/first light efficiency improving layer as described above, and then a compound (Alq₃) having a high refractive index value was employed for the second light efficiency improving layer, and a compound of [Formula I] according to the present invention shown in the following Table 2 having a relatively low refractive index value was employed for the first light efficiency improving layer, and the compounds were film-formed to a total thickness of 65 nm to manufacture an organic light-emitting device.

### Device Comparative Example 2

An organic light-emitting device for Device Comparative Example 2 was manufactured in the same manner as in the device structures in Examples 16 to 69, except that the light efficiency improving layer was not provided.

### Device Comparative Example 3

An organic light-emitting device for Device Comparative Example 3 were manufactured in the same manner as in the device structures of Examples 16 to 69 above, except that [Alq₃] was employed for the second light efficiency improving layer, and the following [CP1-1] was used for the first light efficiency improving layer instead of the [Formula I] compound according to the present invention.

### Device Comparative Example 4

An organic light-emitting device for Device Comparative Example 4 were manufactured in the same manner as in the device structures of Examples 16 to 69 above, except that [Alq₃] was employed for the second light efficiency improving layer, and the following [CP1-2] was used for the first light efficiency improving layer instead of the [Formula I] compound according to the present invention.

### Experimental Example 2: Light-Emitting Properties of Device Examples 16 to 69

For the organic light-emitting devices manufactured by the Examples and the Comparative Examples, driving voltage, current efficiency and color coordinate were measured using a source meter (Model237, Keithley) and a luminance meter (PR-650, Photo Research), and the result values based on 1,000 nits are shown in the following [Table 2].

TABLE 2

| | Second | | | | | |
|---|---|---|---|---|---|---|
| Example | CPL | First CPL | V | cd/A | CIEx | CIEy |
| 16 | Alq3 | Formula 3 | 3.86 | 8.43 | 0.1336 | 0.0582 |
| 17 | | Formula 5 | 4.01 | 8.46 | 0.1347 | 0.0579 |
| 18 | | Formula 11 | 3.70 | 8.47 | 0.1342 | 0.0566 |
| 19 | | Formula 13 | 3.62 | 8.54 | 0.1350 | 0.0558 |
| 20 | | Formula 22 | 3.67 | 8.98 | 0.1323 | 0.0528 |
| 21 | | Formula 27 | 3.72 | 8.31 | 0.1382 | 0.0484 |
| 22 | | Formula 31 | 3.61 | 8.23 | 0.1390 | 0.0466 |
| 23 | | Formula 33 | 3.63 | 8.90 | 0.1379 | 0.0565 |
| 24 | | Formula 36 | 3.71 | 8.50 | 0.1368 | 0.0555 |
| 25 | | Formula 40 | 3.73 | 8.21 | 0.1350 | 0.0481 |
| 26 | | Formula 49 | 3.78 | 8.64 | 0.1375 | 0.0454 |
| 27 | | Formula 56 | 3.64 | 8.81 | 0.1368 | 0.0595 |
| 28 | | Formula 67 | 3.72 | 8.32 | 0.1389 | 0.0537 |
| 29 | | Formula 71 | 3.75 | 8.75 | 0.1307 | 0.0575 |
| 30 | | Formula 73 | 3.74 | 8.28 | 0.1327 | 0.0510 |
| 31 | | Formula 77 | 3.73 | 8.41 | 0.1324 | 0.0575 |
| 32 | | Formula 78 | 3.81 | 8.35 | 0.1328 | 0.0568 |
| 33 | | Formula 79 | 3.57 | 8.34 | 0.1383 | 0.0485 |
| 34 | | Formula 93 | 3.75 | 8.63 | 0.1317 | 0.0618 |
| 35 | | Formula 102 | 3.81 | 8.47 | 0.1351 | 0.0497 |
| 36 | | Formula 105 | 3.87 | 8.42 | 0.1319 | 0.0492 |
| 37 | | Formula 107 | 3.75 | 8.75 | 0.1388 | 0.0481 |
| 38 | | Formula 108 | 3.46 | 8.48 | 0.1329 | 0.0589 |
| 39 | | Formula 118 | 3.49 | 8.33 | 0.1340 | 0.0539 |
| 40 | | Formula 126 | 3.79 | 8.26 | 0.1368 | 0.0442 |
| 41 | | Formula 133 | 3.88 | 8.25 | 0.1332 | 0.0524 |
| 42 | | Formula 138 | 3.91 | 8.23 | 0.1338 | 0.0571 |
| 43 | | Formula 142 | 3.64 | 8.59 | 0.1350 | 0.0499 |
| 44 | | Formula 143 | 3.42 | 8.37 | 0.1346 | 0.0554 |
| 45 | | Formula 144 | 3.76 | 8.34 | 0.1360 | 0.0493 |
| 46 | | Formula 147 | 3.62 | 8.45 | 0.1389 | 0.0565 |
| 47 | | Formula 150 | 3.87 | 8.29 | 0.1375 | 0.0468 |
| 48 | | Formula 152 | 3.93 | 8.38 | 0.1324 | 0.0582 |
| 49 | | Formula 153 | 3.73 | 8.42 | 0.1379 | 0.0492 |
| 50 | | Formula 163 | 3.84 | 8.24 | 0.1349 | 0.0515 |
| 51 | | Formula 168 | 3.76 | 8.31 | 0.1357 | 0.0482 |
| 52 | | Formula 179 | 3.77 | 8.46 | 0.1343 | 0.0553 |
| 53 | | Formula 183 | 3.88 | 8.41 | 0.1329 | 0.0574 |
| 54 | | Formula 185 | 3.90 | 8.28 | 0.1355 | 0.0531 |
| 55 | | Formula 195 | 3.74 | 8.52 | 0.1372 | 0.0552 |
| 56 | | Formula 201 | 3.71 | 8.40 | 0.1319 | 0.0585 |
| 57 | | Formula 204 | 3.67 | 8.59 | 0.1333 | 0.0521 |
| 58 | | Formula 205 | 3.44 | 8.83 | 0.1338 | 0.0527 |
| 59 | | Formula 209 | 3.85 | 8.66 | 0.1343 | 0.0523 |
| 60 | | Formula 213 | 3.75 | 8.37 | 0.1325 | 0.0584 |
| 61 | | Formula 219 | 3.95 | 8.57 | 0.1371 | 0.0484 |
| 62 | | Formula 226 | 3.75 | 8.36 | 0.1316 | 0.0536 |
| 63 | | Formula 231 | 3.53 | 8.41 | 0.1357 | 0.0504 |
| 64 | | Formula 238 | 3.57 | 8.39 | 0.1358 | 0.0496 |
| 65 | | Formula 243 | 3.91 | 8.31 | 0.1345 | 0.0470 |
| 66 | | Formula 256 | 3.96 | 8.50 | 0.1378 | 0.0489 |
| 67 | | Formula 261 | 3.87 | 8.42 | 0.1367 | 0.0462 |
| 68 | | Formula 269 | 3.99 | 8.28 | 0.1319 | 0.0621 |
| 69 | | Formula 271 | 3.74 | 8.42 | 0.1371 | 0.0550 |
| Comparative | Light efficiency | | 4.68 | 7.03 | 0.1502 | 0.1412 |
| Example 2 | improving layer | | | | | |
| | not provided | | | | | |
| Comparative | Alq₃ | CP 1-1 | 4.37 | 7.76 | 0.1407 | 0.0713 |
| Example 3 | | | | | | |
| Comparative | Alq₃ | CP 1-2 | 4.26 | 8.06 | 0.1350 | 0.0685 |
| Example 4 | | | | | | |

As shown in [Table 2] above, it can be confirmed that while configuring the light efficiency improving layer provided in the organic light-emitting device according to the embodiments of the present invention as a plurality of layers (second light efficiency improving layer/first light efficiency improving layer) using compounds having different refractive index values, and employing a compound (Alq₃) in the related art having a high refractive index value for the second light efficiency improving layer, a device, in which the [Formula I] compound according to the present invention having a relatively low refractive index value is employed for the first light efficiency improving layer, has improved driving voltage, light-emitting efficiency, and color purity properties compared to each of the device not provided with the light efficiency improving layer (Comparative Example 2) and the devices in which a compound in contrast with the structural characteristics of the [Formula I] compound according to the present invention is employed for the second light efficiency improving layer (Comparative Examples 3 and 4).

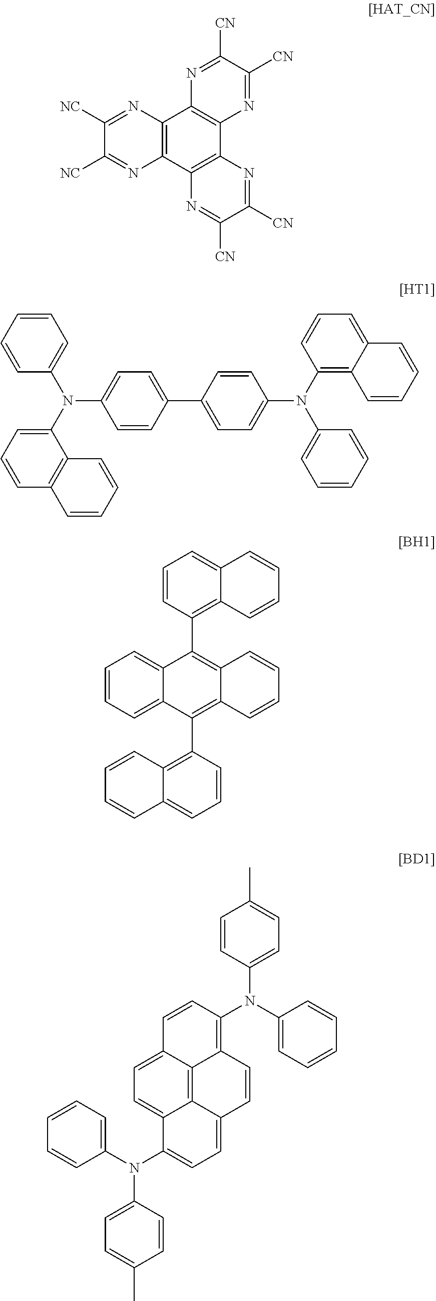

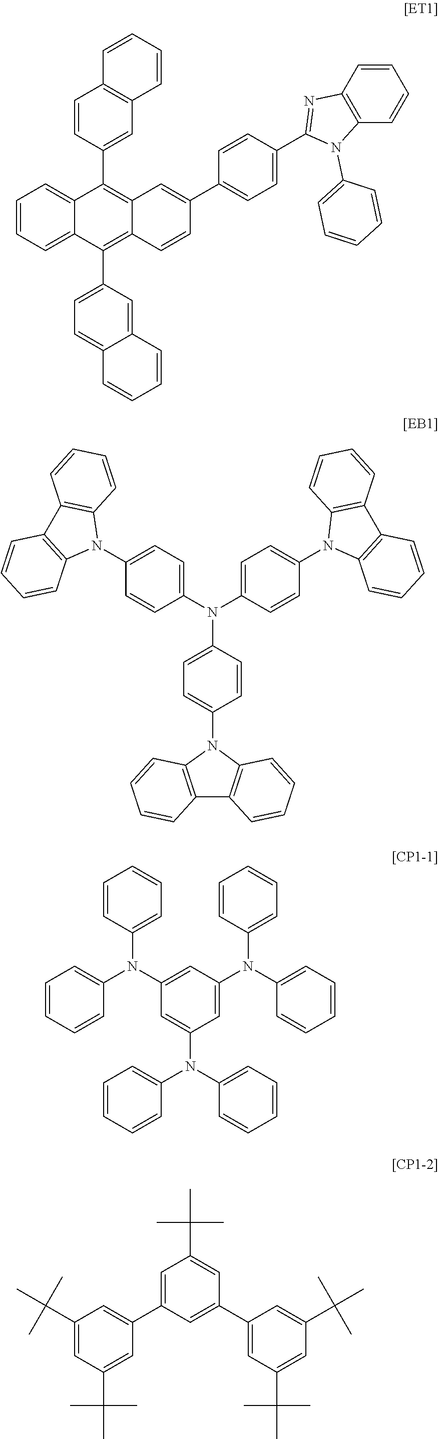

### INDUSTRIAL APPLICABILITY

The organic compound according to the present invention has a low refractive index and can improve the efficiency of light extracted to the outside of an organic light emitting device, and thus can be usefully used as a material for a light efficiency improving layer provided in the organic light emitting device. Accordingly, by employing the compound according to the present invention in the light efficiency improving layer, it is possible to implement a highly efficient and long service life organic light-emitting device having improved light-emitting efficiency, color purity, service life characteristics, as well as low voltage driving characteristics, and the like, so the organic light-emitting device can be industrially and usefully used in various lighting and display devices, and the like.

1. An organic compound represented by Formula I below: 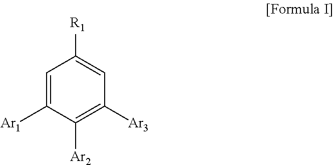
wherein, in [Formula I] above,
R₁ is any one selected from a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atom, and the following [Structural Formula 1],
Ar₁ to Ar₃ are the same as or different from each other, and each independently any one selected from a substituted or unsubstituted aryl group having 6 to 30 carbon atoms, a substituted or unsubstituted heteroaryl group having 2 to 30 carbon atoms, and the following [Structural Formula 1], 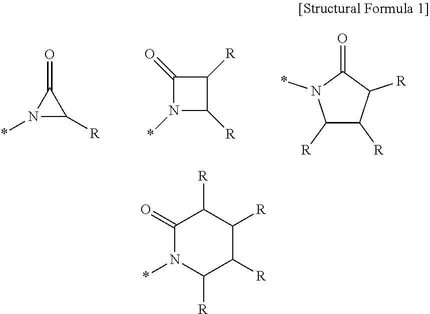
in Structural Formula 1 above,
R is each independently any one selected from hydrogen, deuterium, a cyano group, a halogen group, a substituted or unsubstituted alkyl group having 1 to 20 carbon atoms, a substituted or unsubstituted halogenated alkyl group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms.

2. The organic compound of claim 1, wherein, in the definitions of R, R₁, and Ar₁ to Ar₃, ‘substituted or unsubstituted’ means that each of R, R₁, and Ar₁ to Ar₃ is substituted with one or two or more substituents selected from the group consisting of deuterium, a halogen group, a cyano group, a nitro group, a hydroxyl group, a silyl group, an amine group, a halogenated alkyl group, a deuterated alkyl group, a cycloalkyl group, a heterocycloalkyl group, an aryl group, a heteroaryl group, an alkylsilyl group, an arylsilyl group, and the following [Structural Formula 1], is substituted with a substituent to which two or more substituent among the above substituents are linked, or has no substituent: 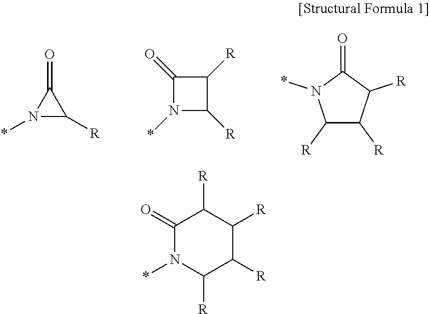
in [Structural Formula 1] above,
R is each independently any one selected from hydrogen, deuterium, a cyano group, a halogen group, an alkyl group having 1 to 20 carbon atoms, an alkyl group having 1 to 20 carbon atoms, a halogenated alkyl group having 1 to 20 carbon atoms, a cycloalkyl group having 3 to 20 carbon atoms, an aryl group having 6 to 30 carbon atoms, and a heteroaryl group having 2 to 30 carbon atoms.

3. The organic compound of claim 1, wherein [Formula I] above is any one selected from the following [Compound 1] to [Compound 271]:

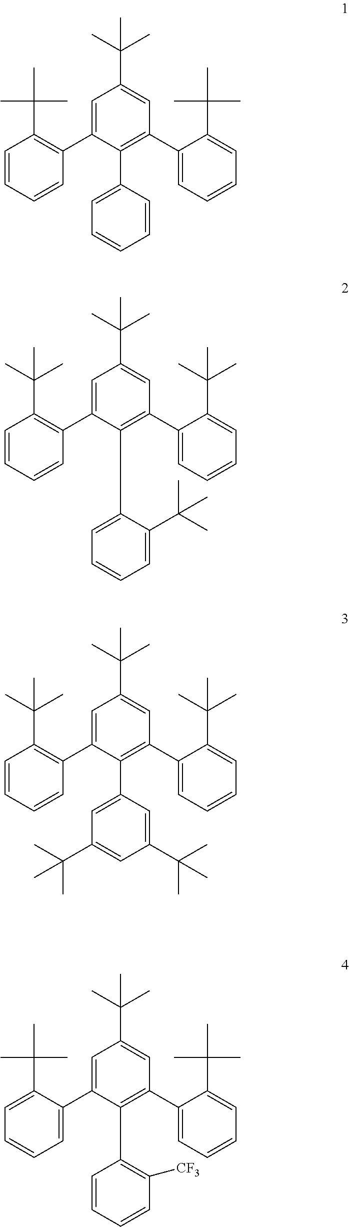

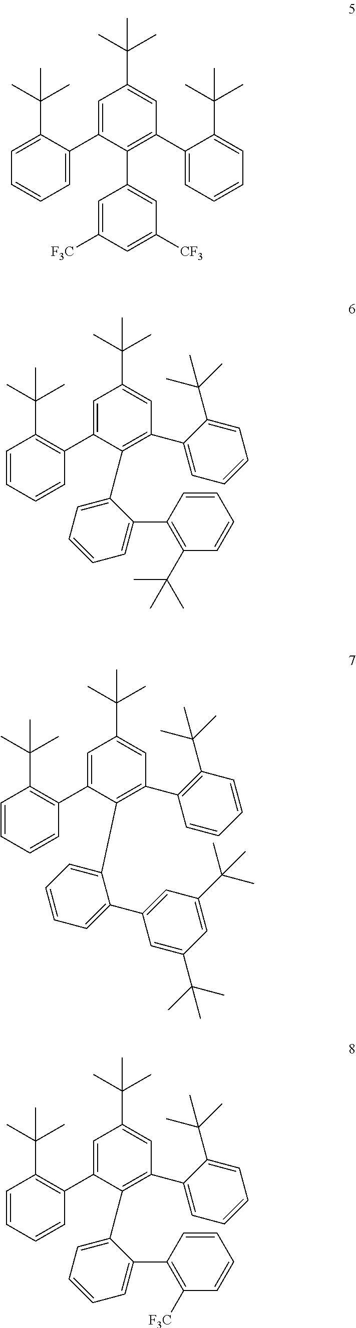

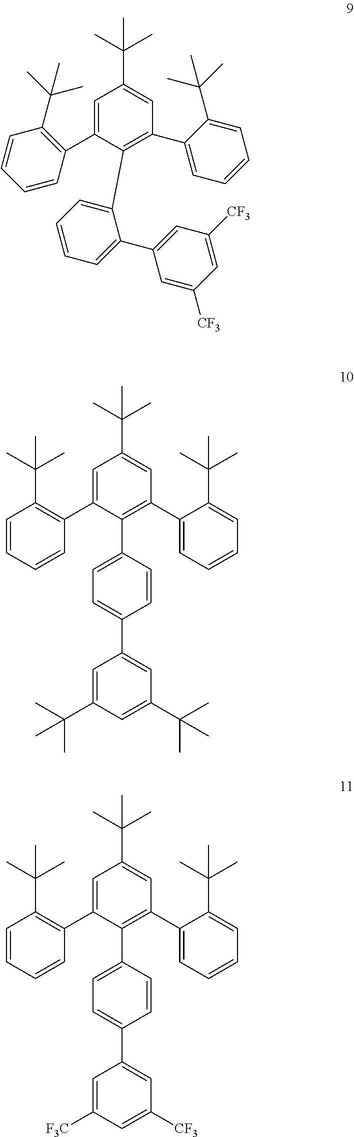

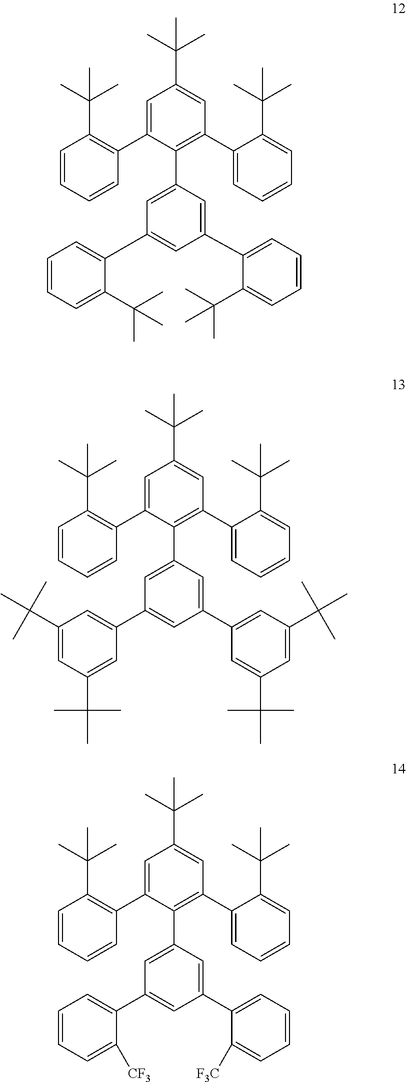

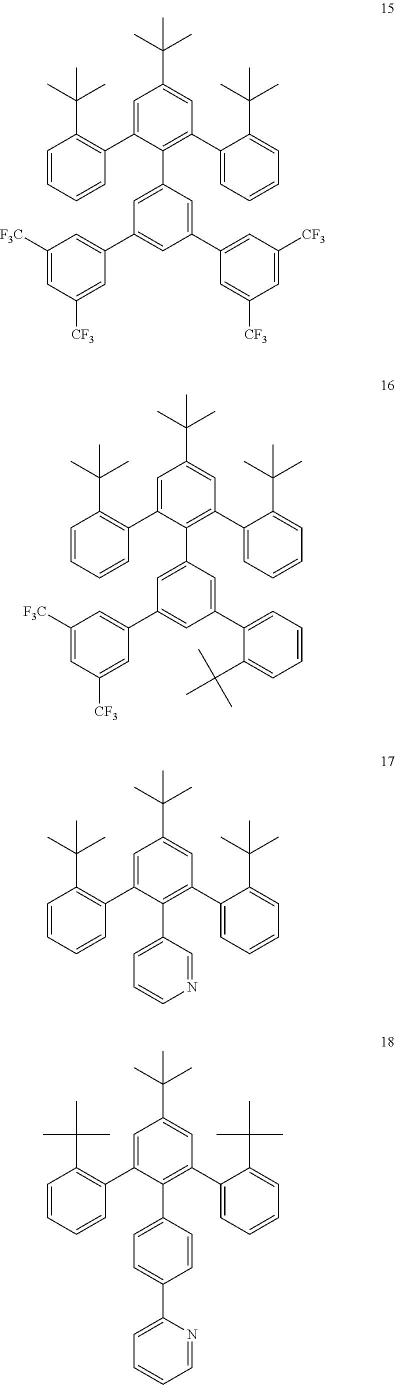

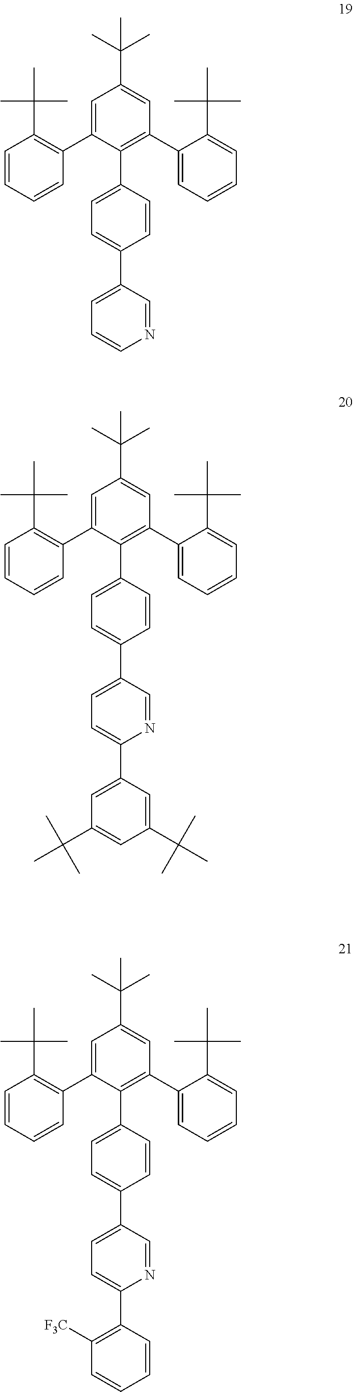

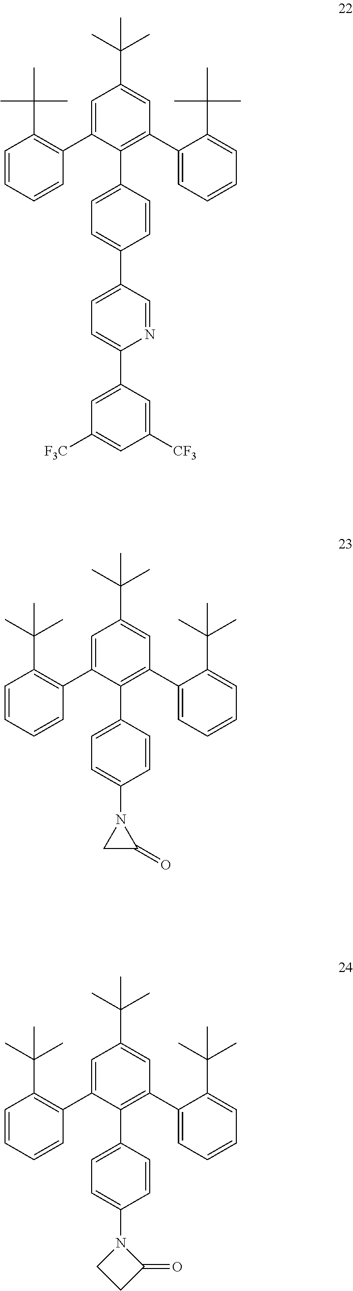 [SMILES: CC(C)(C)c1cc(-c2ccccc2C(C)(C)C)c(-c2ccc(-c3ccc(-c4cc(C(F)(F)F)cc(C(F)(F)F)c4)nc3)cc2)c(-c2ccccc2C(C)(C)C)c1 ; CC(C)(C)c1cc(-c2ccccc2C(C)(C)C)c(-c2ccc(N3CC3=O)cc2)c(-c2ccccc2C(C)(C)C)c1 ; CC(C)(C)c1cc(-c2ccccc2C(C)(C)C)c(-c2ccc(N3CCC3=O)cc2)c(-c2ccccc2C(C)(C)C)c1]

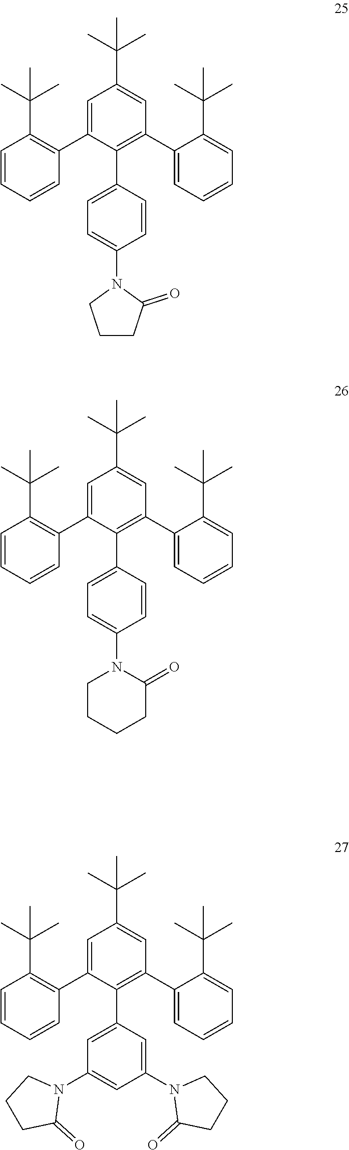 [SMILES: CC(C)(C)c1cc(-c2ccccc2C(C)(C)C)c(-c2ccc(N3CCCC3=O)cc2)c(-c2ccccc2C(C)(C)C)c1 ; CC(C)(C)c1cc(-c2ccccc2C(C)(C)C)c(-c2ccc(N3CCCCC3=O)cc2)c(-c2ccccc2C(C)(C)C)c1 ; CC(C)(C)c1cc(-c2ccccc2C(C)(C)C)c(-c2cc(N3CCCC3=O)cc(N3CCCC3=O)c2)c(-c2ccccc2C(C)(C)C)c1]

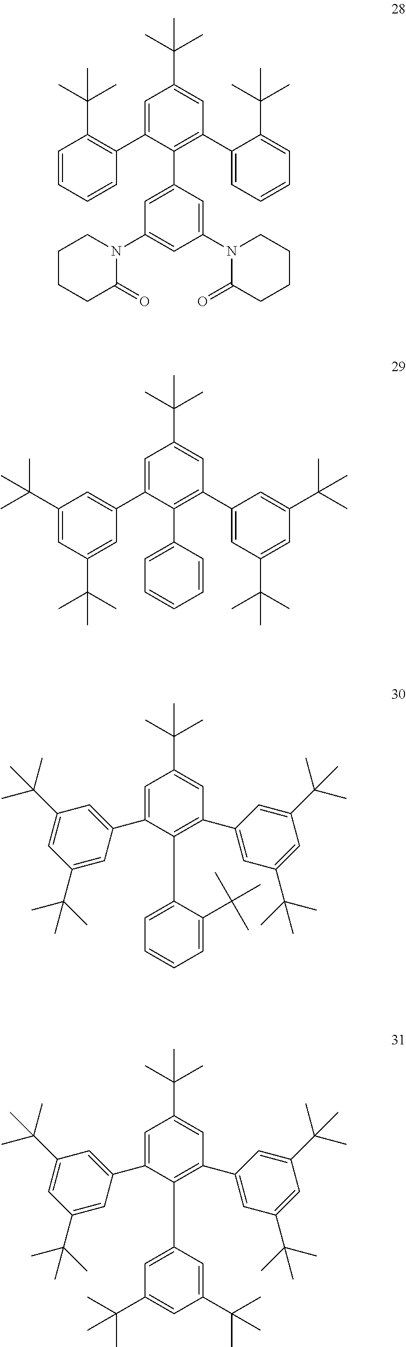

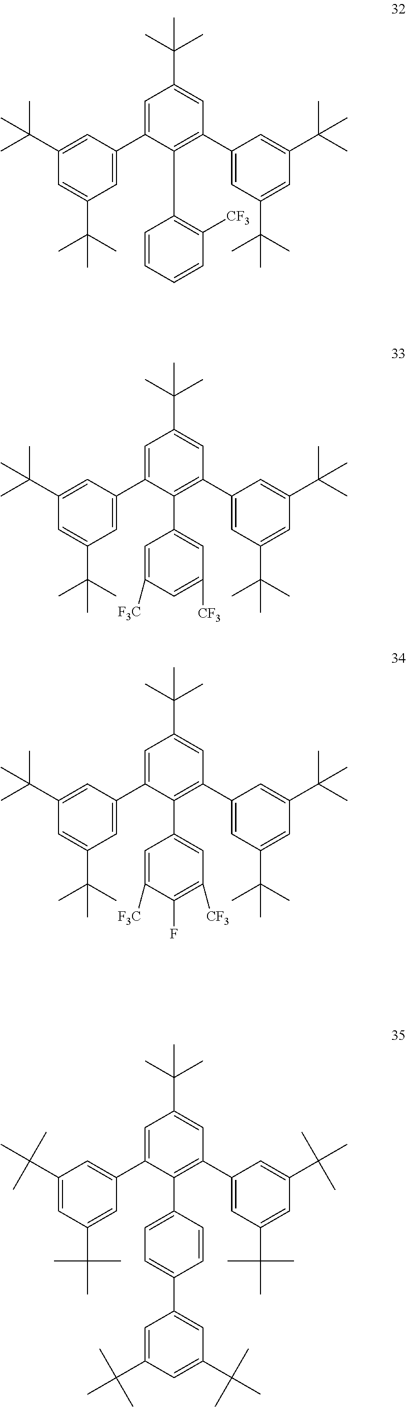

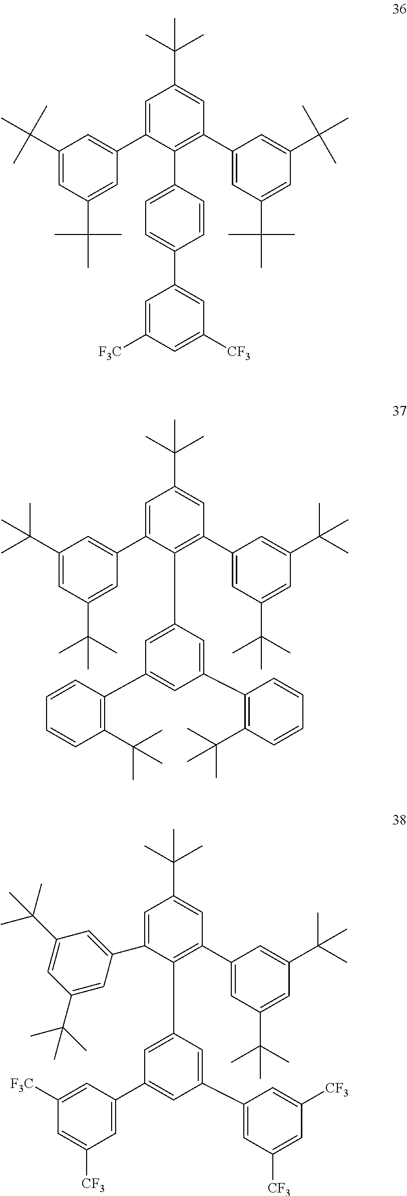

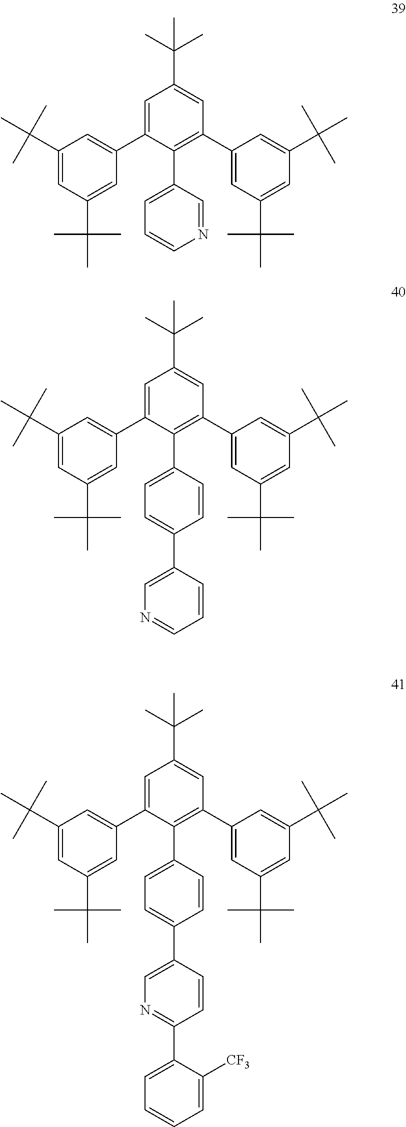

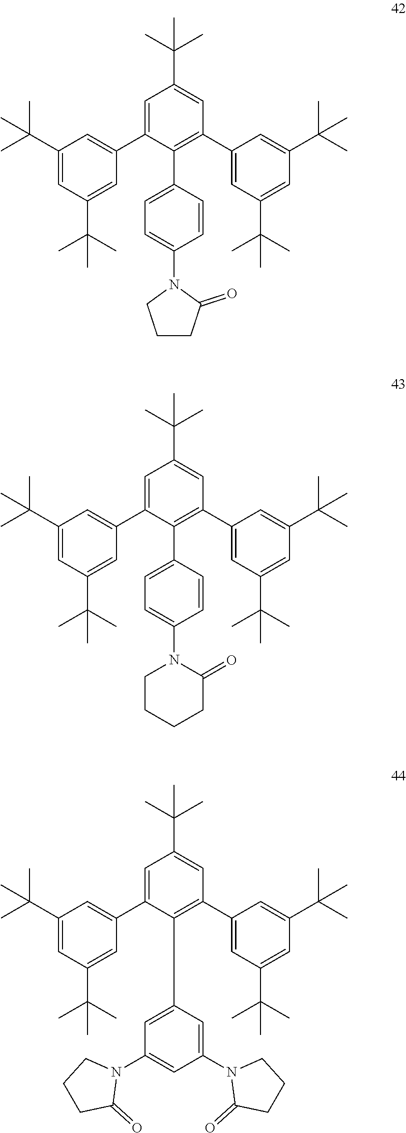

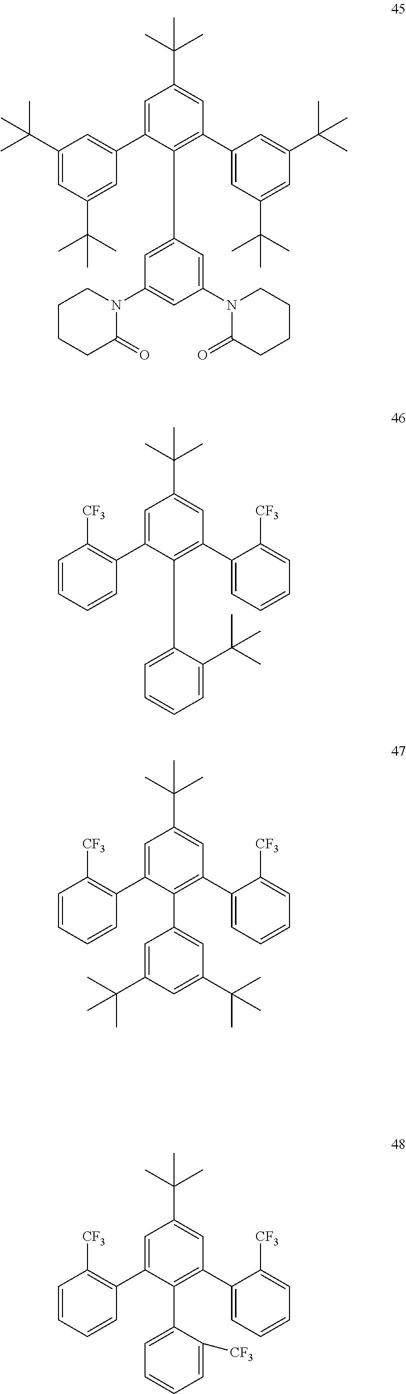

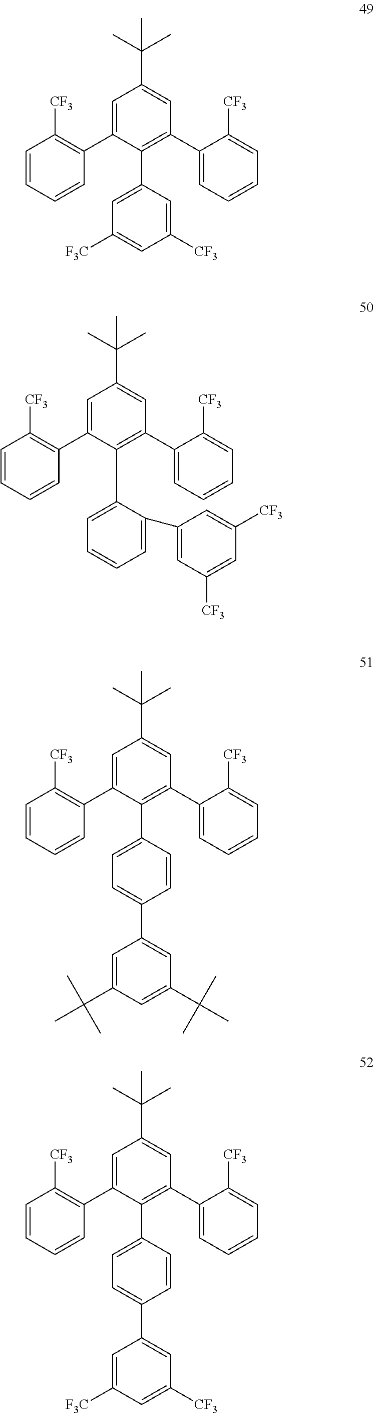 [SMILES: CC(C)(C)c1cc(-c2ccccc2C(F)(F)F)c(-c2cc(C(F)(F)F)cc(C(F)(F)F)c2)c(-c2ccccc2C(F)(F)F)c1 ; CC(C)(C)c1cc(-c2ccccc2C(F)(F)F)c(-c2ccccc2-c2cc(C(F)(F)F)cc(C(F)(F)F)c2)c(-c2ccccc2C(F)(F)F)c1 ; CC(C)(C)c1cc(-c2ccc(-c3c(-c4ccccc4C(F)(F)F)cc(C(C)(C)C)cc3-c3ccccc3C(F)(F)F)cc2)cc(C(C)(C)C)c1 ; CC(C)(C)c1cc(-c2ccccc2C(F)(F)F)c(-c2ccc(-c3cc(C(F)(F)F)cc(C(F)(F)F)c3)cc2)c(-c2ccccc2C(F)(F)F)c1]

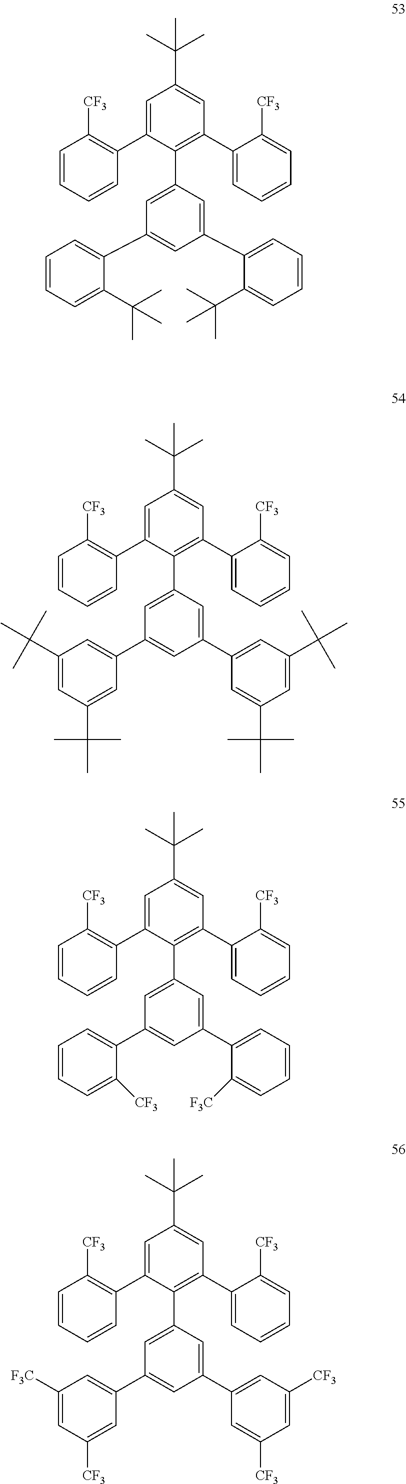 [SMILES: CC(C)(C)c1cc(-c2ccccc2C(F)(F)F)c(-c2cc(-c3ccccc3C(C)(C)C)cc(-c3ccccc3C(C)(C)C)c2)c(-c2ccccc2C(F)(F)F)c1 ; CC(C)(C)c1cc(-c2cc(-c3cc(C(C)(C)C)cc(C(C)(C)C)c3)cc(-c3c(-c4ccccc4C(F)(F)F)cc(C(C)(C)C)cc3-c3ccccc3C(F)(F)F)c2)cc(C(C)(C)C)c1 ; CC(C)(C)c1cc(-c2ccccc2C(F)(F)F)c(-c2cc(-c3ccccc3C(F)(F)F)cc(-c3ccccc3C(F)(F)F)c2)c(-c2ccccc2C(F)(F)F)c1 ; CC(C)(C)c1cc(-c2ccccc2C(F)(F)F)c(-c2cc(-c3cc(C(F)(F)F)cc(C(F)(F)F)c3)cc(-c3cc(C(F)(F)F)cc(C(F)(F)F)c3)c2)c(-c2ccccc2C(F)(F)F)c1]

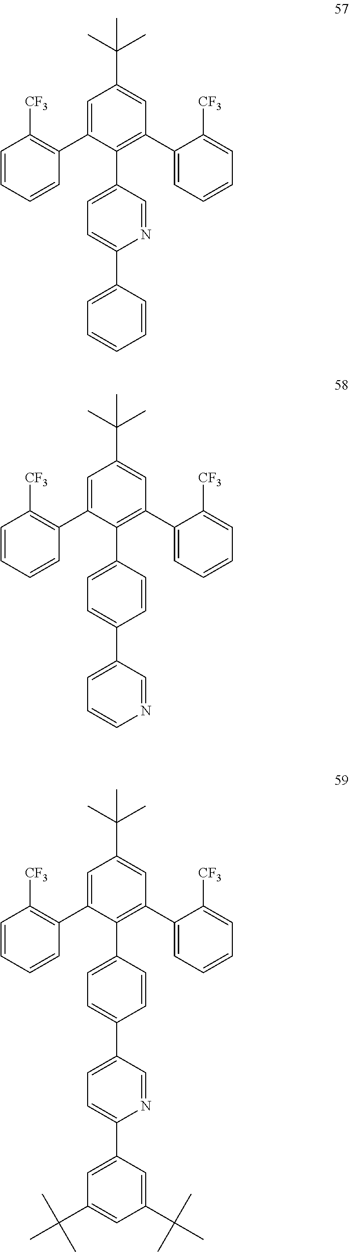

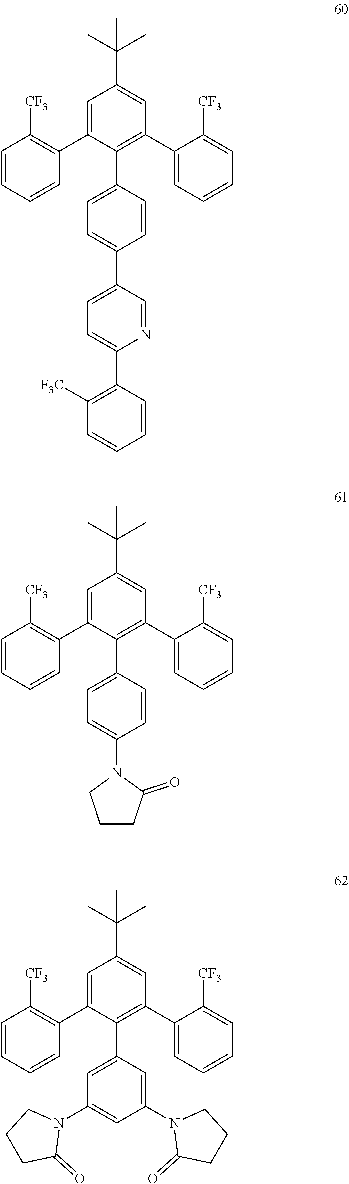 [SMILES: CC(C)(C)c1cc(-c2ccccc2C(F)(F)F)c(-c2ccc(-c3ccc(-c4ccccc4C(F)(F)F)nc3)cc2)c(-c2ccccc2C(F)(F)F)c1 ; CC(C)(C)c1cc(-c2ccccc2C(F)(F)F)c(-c2ccc(N3CCCC3=O)cc2)c(-c2ccccc2C(F)(F)F)c1 ; CC(C)(C)c1cc(-c2ccccc2C(F)(F)F)c(-c2cc(N3CCCC3=O)cc(N3CCCC3=O)c2)c(-c2ccccc2C(F)(F)F)c1]

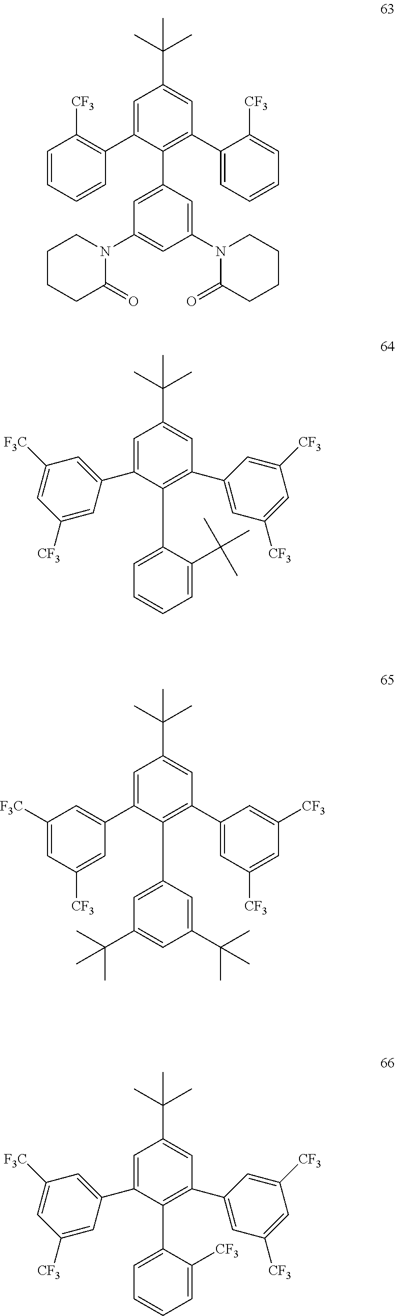 [SMILES: CC(C)(C)c1cc(-c2ccccc2C(F)(F)F)c(-c2cc(N3CCCCC3=O)cc(N3CCCCC3=O)c2)c(-c2ccccc2C(F)(F)F)c1 ; CC(C)(C)c1cc(-c2cc(C(F)(F)F)cc(C(F)(F)F)c2)c(-c2ccccc2C(C)(C)C)c(-c2cc(C(F)(F)F)cc(C(F)(F)F)c2)c1 ; CC(C)(C)c1cc(-c2c(-c3cc(C(F)(F)F)cc(C(F)(F)F)c3)cc(C(C)(C)C)cc2-c2cc(C(F)(F)F)cc(C(F)(F)F)c2)cc(C(C)(C)C)c1 ; CC(C)(C)c1cc(-c2cc(C(F)(F)F)cc(C(F)(F)F)c2)c(-c2ccccc2C(F)(F)F)c(-c2cc(C(F)(F)F)cc(C(F)(F)F)c2)c1]

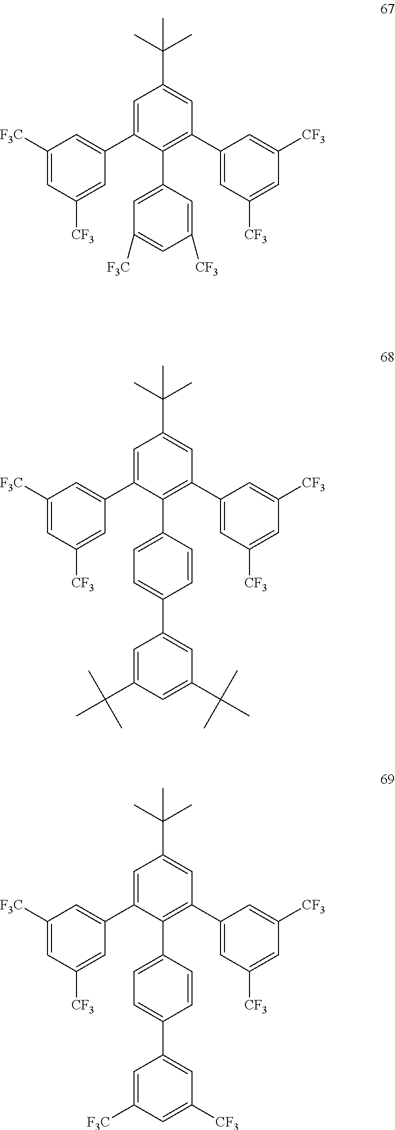 [SMILES: CC(C)(C)c1cc(-c2cc(C(F)(F)F)cc(C(F)(F)F)c2)c(-c2cc(C(F)(F)F)cc(C(F)(F)F)c2)c(-c2cc(C(F)(F)F)cc(C(F)(F)F)c2)c1 ; CC(C)(C)c1cc(-c2ccc(-c3c(-c4cc(C(F)(F)F)cc(C(F)(F)F)c4)cc(C(C)(C)C)cc3-c3cc(C(F)(F)F)cc(C(F)(F)F)c3)cc2)cc(C(C)(C)C)c1 ; CC(C)(C)c1cc(-c2cc(C(F)(F)F)cc(C(F)(F)F)c2)c(-c2ccc(-c3cc(C(F)(F)F)cc(C(F)(F)F)c3)cc2)c(-c2cc(C(F)(F)F)cc(C(F)(F)F)c2)c1]

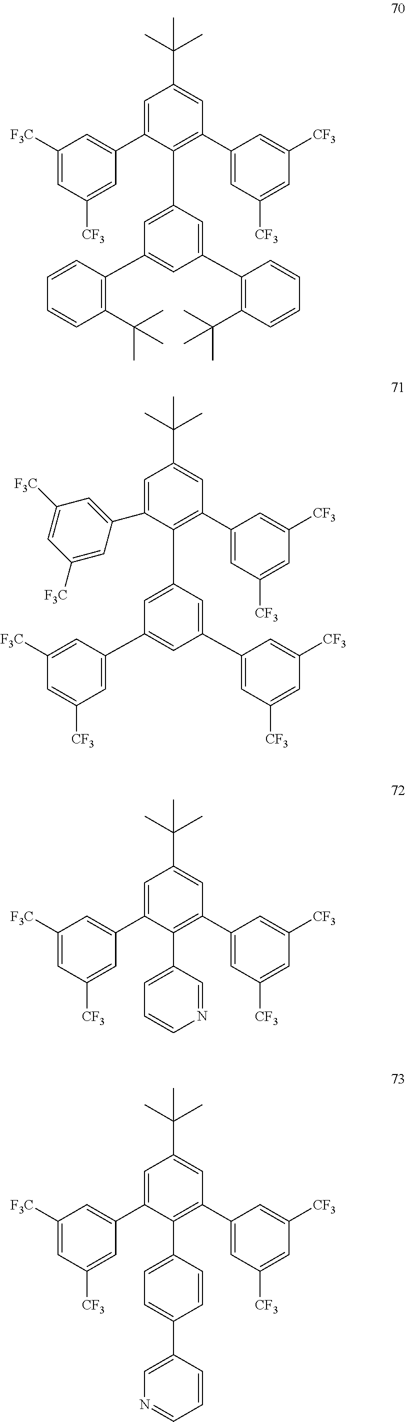 [SMILES: CC(C)(C)c1cc(-c2cc(C(F)(F)F)cc(C(F)(F)F)c2)c(-c2cc(-c3ccccc3C(C)(C)C)cc(-c3ccccc3C(C)(C)C)c2)c(-c2cc(C(F)(F)F)cc(C(F)(F)F)c2)c1 ; CC(C)(C)c1cc(-c2cc(C(F)(F)F)cc(C(F)(F)F)c2)c(-c2cc(-c3cc(C(F)(F)F)cc(C(F)(F)F)c3)cc(-c3cc(C(F)(F)F)cc(C(F)(F)F)c3)c2)c(-c2cc(C(F)(F)F)cc(C(F)(F)F)c2)c1 ; CC(C)(C)c1cc(-c2cc(C(F)(F)F)cc(C(F)(F)F)c2)c(-c2cccnc2)c(-c2cc(C(F)(F)F)cc(C(F)(F)F)c2)c1 ; CC(C)(C)c1cc(-c2cc(C(F)(F)F)cc(C(F)(F)F)c2)c(-c2ccc(-c3cccnc3)cc2)c(-c2cc(C(F)(F)F)cc(C(F)(F)F)c2)c1]

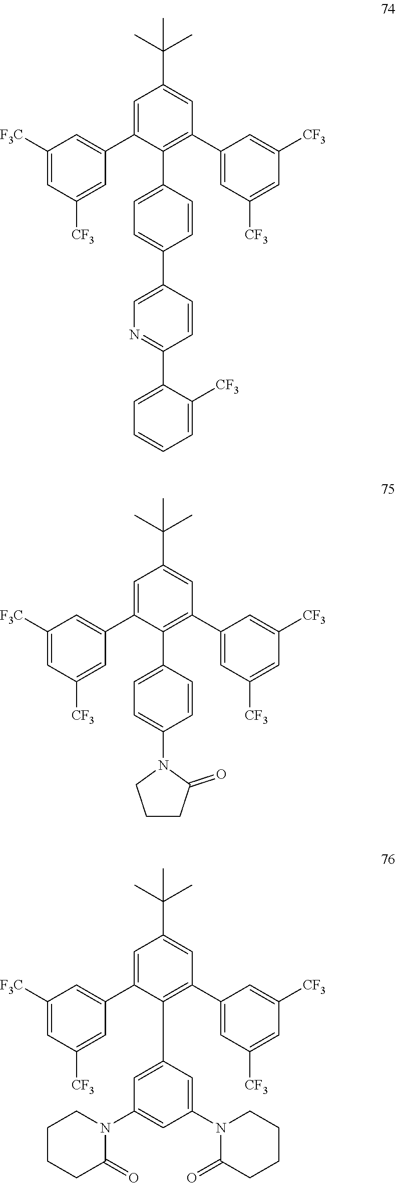 [SMILES: CC(C)(C)c1cc(-c2cc(C(F)(F)F)cc(C(F)(F)F)c2)c(-c2ccc(-c3ccc(-c4ccccc4C(F)(F)F)nc3)cc2)c(-c2cc(C(F)(F)F)cc(C(F)(F)F)c2)c1 ; CC(C)(C)c1cc(-c2cc(C(F)(F)F)cc(C(F)(F)F)c2)c(-c2ccc(N3CCCC3=O)cc2)c(-c2cc(C(F)(F)F)cc(C(F)(F)F)c2)c1 ; CC(C)(C)c1cc(-c2cc(C(F)(F)F)cc(C(F)(F)F)c2)c(-c2cc(N3CCCCC3=O)cc(N3CCCCC3=O)c2)c(-c2cc(C(F)(F)F)cc(C(F)(F)F)c2)c1]

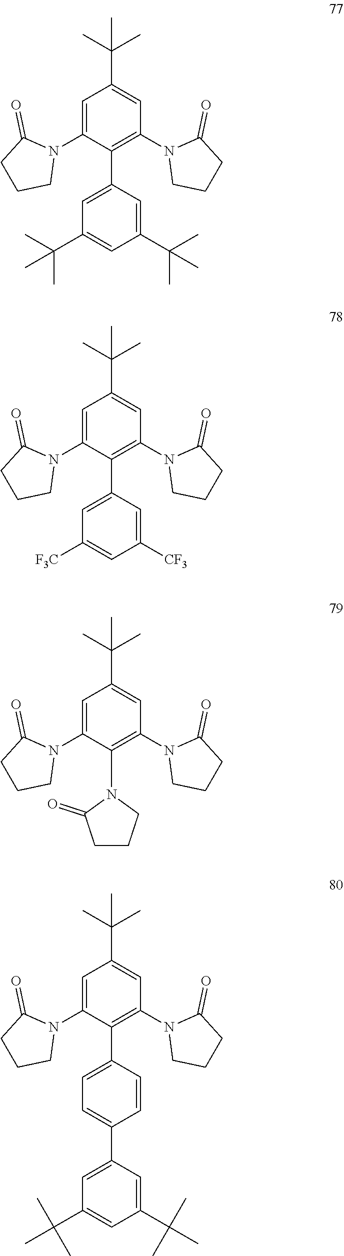 [SMILES: CC(C)(C)c1cc(-c2c(N3CCCC3=O)cc(C(C)(C)C)cc2N2CCCC2=O)cc(C(C)(C)C)c1 ; CC(C)(C)c1cc(N2CCCC2=O)c(-c2cc(C(F)(F)F)cc(C(F)(F)F)c2)c(N2CCCC2=O)c1 ; CC(C)(C)c1cc(N2CCCC2=O)c(N2CCCC2=O)c(N2CCCC2=O)c1 ; CC(C)(C)c1cc(-c2ccc(-c3c(N4CCCC4=O)cc(C(C)(C)C)cc3N3CCCC3=O)cc2)cc(C(C)(C)C)c1]

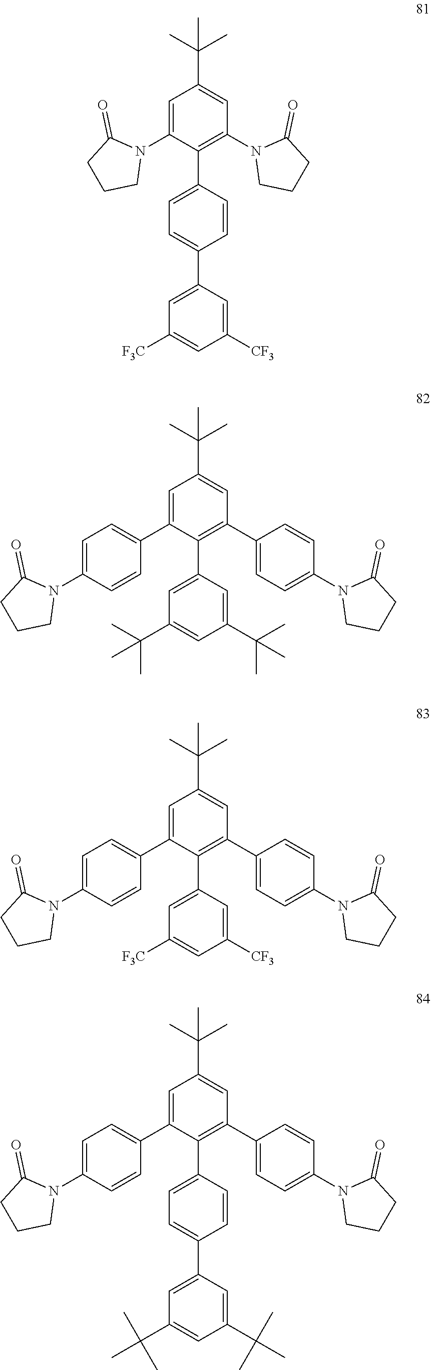 [SMILES: CC(C)(C)c1cc(N2CCCC2=O)c(-c2ccc(-c3cc(C(F)(F)F)cc(C(F)(F)F)c3)cc2)c(N2CCCC2=O)c1 ; CC(C)(C)c1cc(-c2c(-c3ccc(N4CCCC4=O)cc3)cc(C(C)(C)C)cc2-c2ccc(N3CCCC3=O)cc2)cc(C(C)(C)C)c1 ; CC(C)(C)c1cc(-c2ccc(N3CCCC3=O)cc2)c(-c2cc(C(F)(F)F)cc(C(F)(F)F)c2)c(-c2ccc(N3CCCC3=O)cc2)c1 ; CC(C)(C)c1cc(-c2ccc(-c3c(-c4ccc(N5CCCC5=O)cc4)cc(C(C)(C)C)cc3-c3ccc(N4CCCC4=O)cc3)cc2)cc(C(C)(C)C)c1]

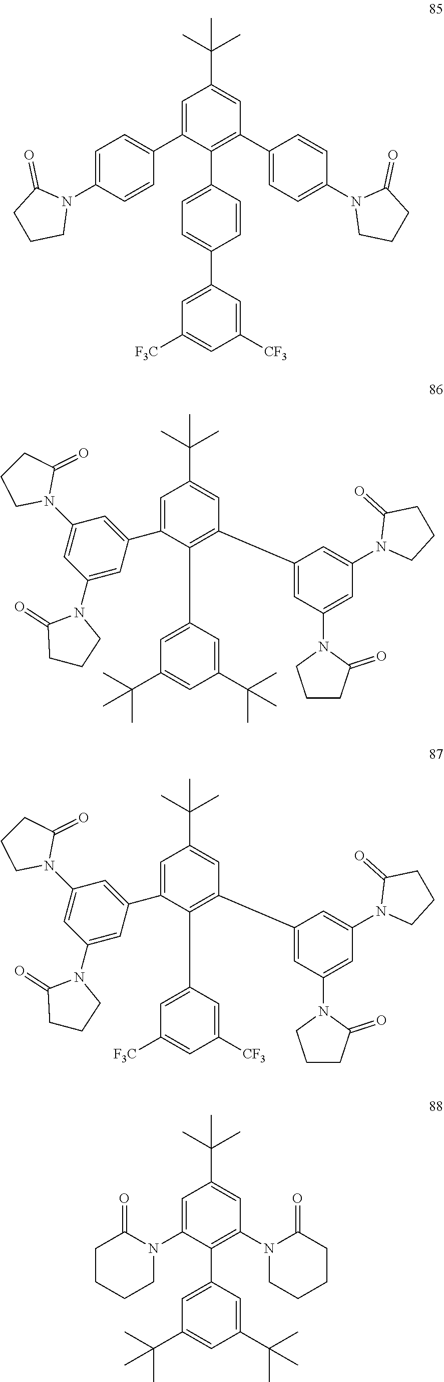 [SMILES: CC(C)(C)c1cc(-c2ccc(N3CCCC3=O)cc2)c(-c2ccc(-c3cc(C(F)(F)F)cc(C(F)(F)F)c3)cc2)c(-c2ccc(N3CCCC3=O)cc2)c1 ; CC(C)(C)c1cc(-c2c(-c3cc(N4CCCC4=O)cc(N4CCCC4=O)c3)cc(C(C)(C)C)cc2-c2cc(N3CCCC3=O)cc(N3CCCC3=O)c2)cc(C(C)(C)C)c1 ; CC(C)(C)c1cc(-c2cc(N3CCCC3=O)cc(N3CCCC3=O)c2)c(-c2cc(C(F)(F)F)cc(C(F)(F)F)c2)c(-c2cc(N3CCCC3=O)cc(N3CCCC3=O)c2)c1 ; CC(C)(C)c1cc(-c2c(N3CCCCC3=O)cc(C(C)(C)C)cc2N2CCCCC2=O)cc(C(C)(C)C)c1]

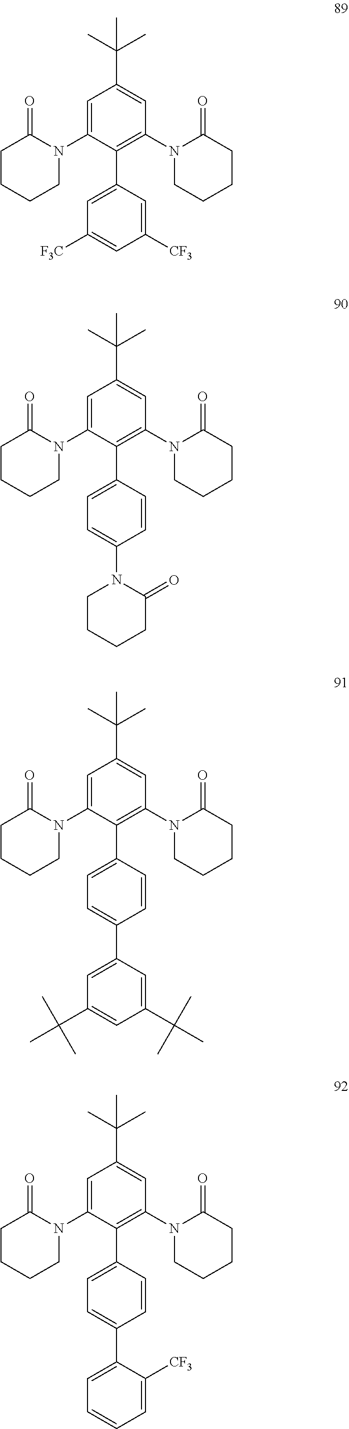

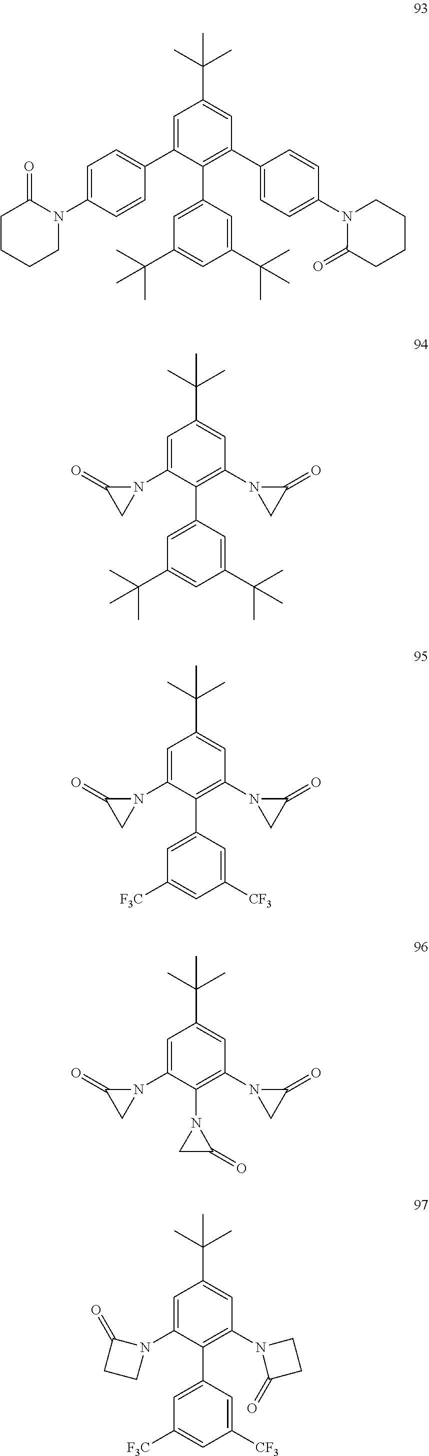 [SMILES: CC(C)(C)c1cc(-c2c(-c3ccc(N4CCCCC4=O)cc3)cc(C(C)(C)C)cc2-c2ccc(N3CCCCC3=O)cc2)cc(C(C)(C)C)c1 ; CC(C)(C)c1cc(-c2c(N3CC3=O)cc(C(C)(C)C)cc2N2CC2=O)cc(C(C)(C)C)c1 ; CC(C)(C)c1cc(N2CC2=O)c(-c2cc(C(F)(F)F)cc(C(F)(F)F)c2)c(N2CC2=O)c1 ; CC(C)(C)c1cc(N2CC2=O)c(N2CC2=O)c(N2CC2=O)c1 ; CC(C)(C)c1cc(N2CCC2=O)c(-c2cc(C(F)(F)F)cc(C(F)(F)F)c2)c(N2CCC2=O)c1]

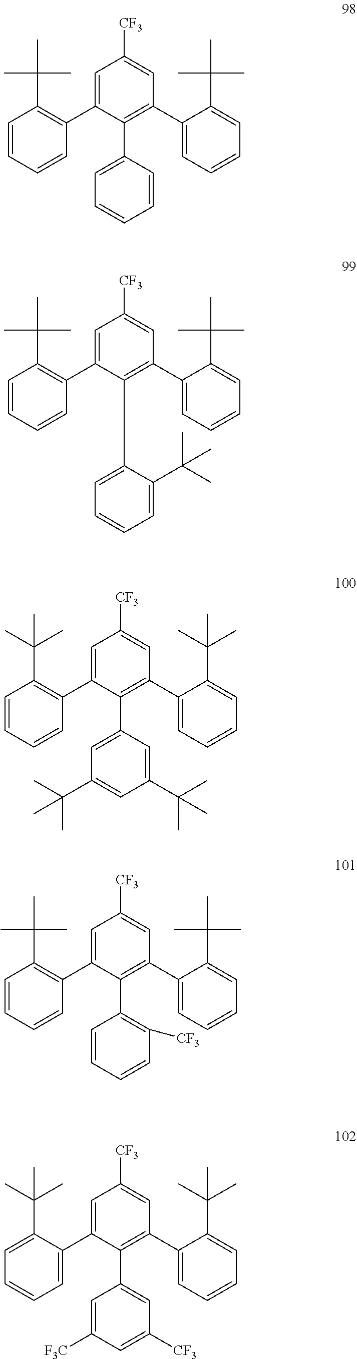

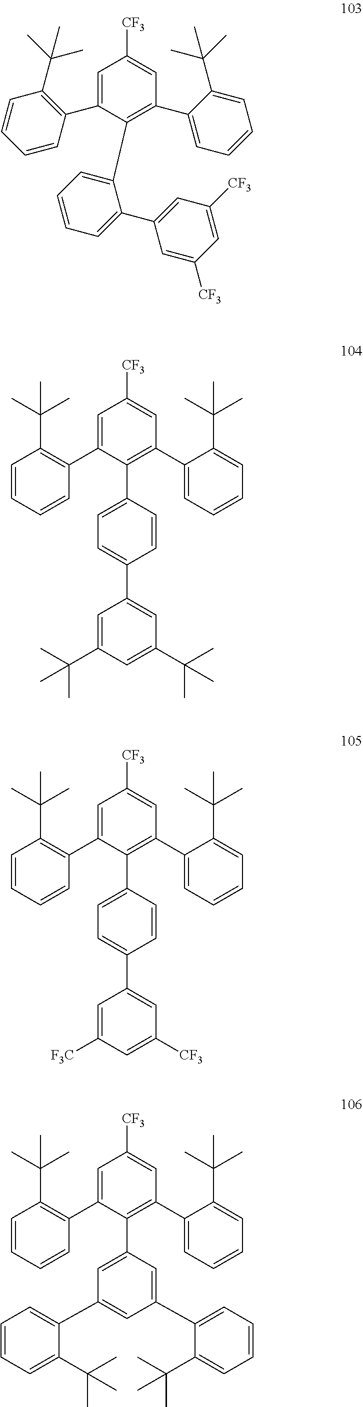 [SMILES: CC(C)(C)c1ccccc1-c1cc(C(F)(F)F)cc(-c2ccccc2C(C)(C)C)c1-c1ccccc1-c1cc(C(F)(F)F)cc(C(F)(F)F)c1 ; CC(C)(C)c1cc(-c2ccc(-c3c(-c4ccccc4C(C)(C)C)cc(C(F)(F)F)cc3-c3ccccc3C(C)(C)C)cc2)cc(C(C)(C)C)c1 ; CC(C)(C)c1ccccc1-c1cc(C(F)(F)F)cc(-c2ccccc2C(C)(C)C)c1-c1ccc(-c2cc(C(F)(F)F)cc(C(F)(F)F)c2)cc1 ; CC(C)(C)c1ccccc1-c1cc(-c2ccccc2C(C)(C)C)cc(-c2c(-c3ccccc3C(C)(C)C)cc(C(F)(F)F)cc2-c2ccccc2C(C)(C)C)c1]

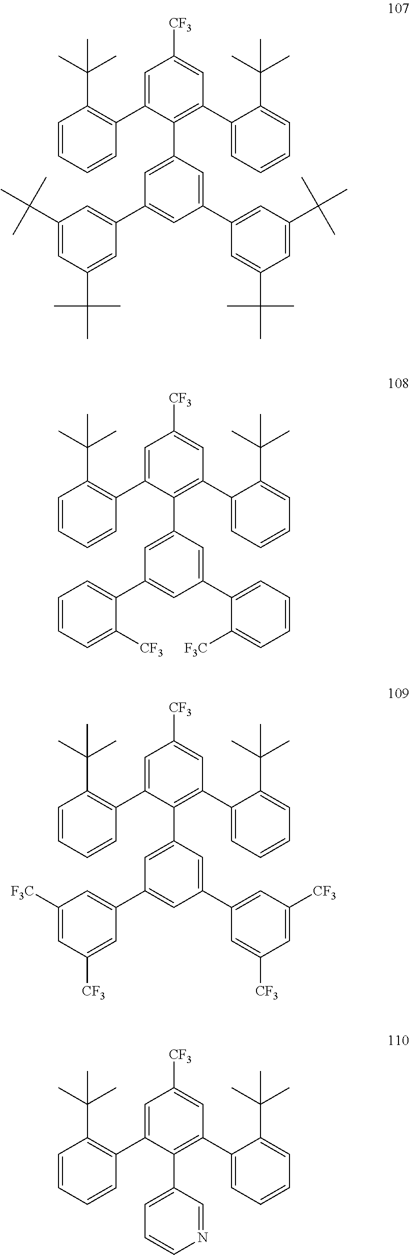 [SMILES: CC(C)(C)c1cc(-c2cc(-c3cc(C(C)(C)C)cc(C(C)(C)C)c3)cc(-c3c(-c4ccccc4C(C)(C)C)cc(C(F)(F)F)cc3-c3ccccc3C(C)(C)C)c2)cc(C(C)(C)C)c1 ; CC(C)(C)c1ccccc1-c1cc(C(F)(F)F)cc(-c2ccccc2C(C)(C)C)c1-c1cc(-c2ccccc2C(F)(F)F)cc(-c2ccccc2C(F)(F)F)c1 ; CC(C)(C)c1ccccc1-c1cc(C(F)(F)F)cc(-c2ccccc2C(C)(C)C)c1-c1cc(-c2cc(C(F)(F)F)cc(C(F)(F)F)c2)cc(-c2cc(C(F)(F)F)cc(C(F)(F)F)c2)c1 ; CC(C)(C)c1ccccc1-c1cc(C(F)(F)F)cc(-c2ccccc2C(C)(C)C)c1-c1cccnc1]

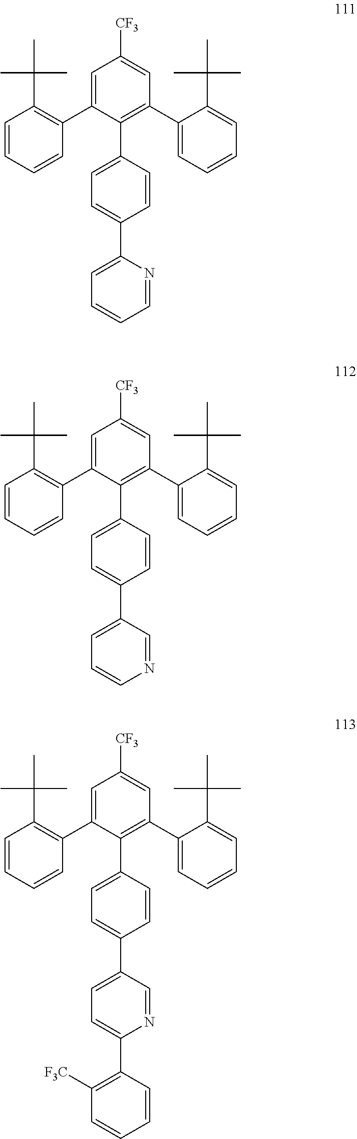

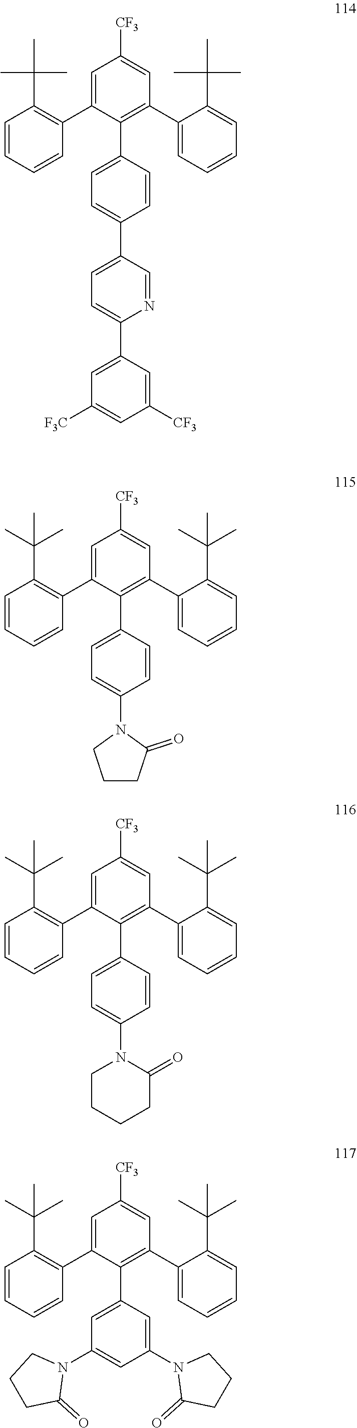

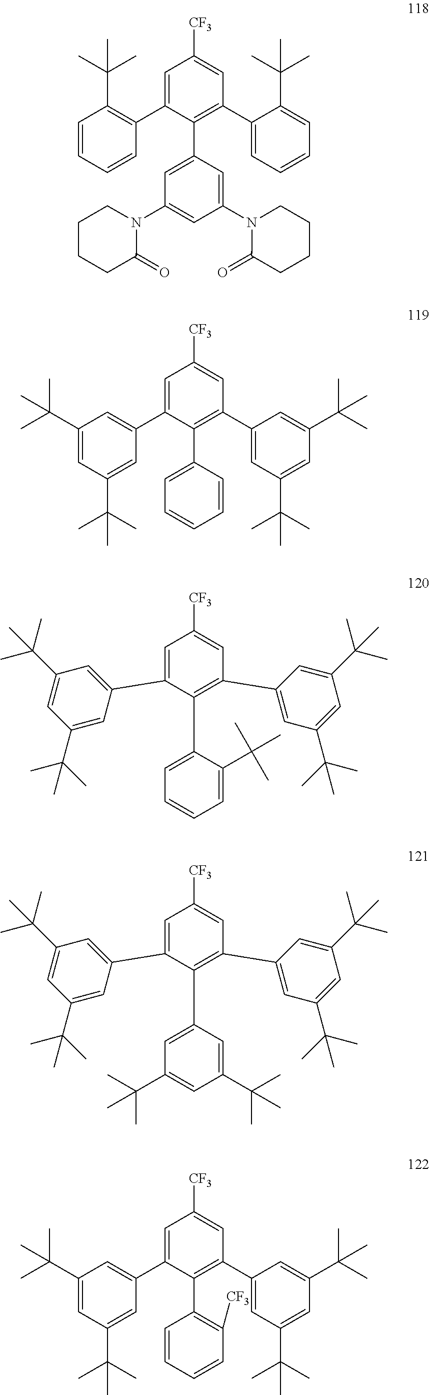

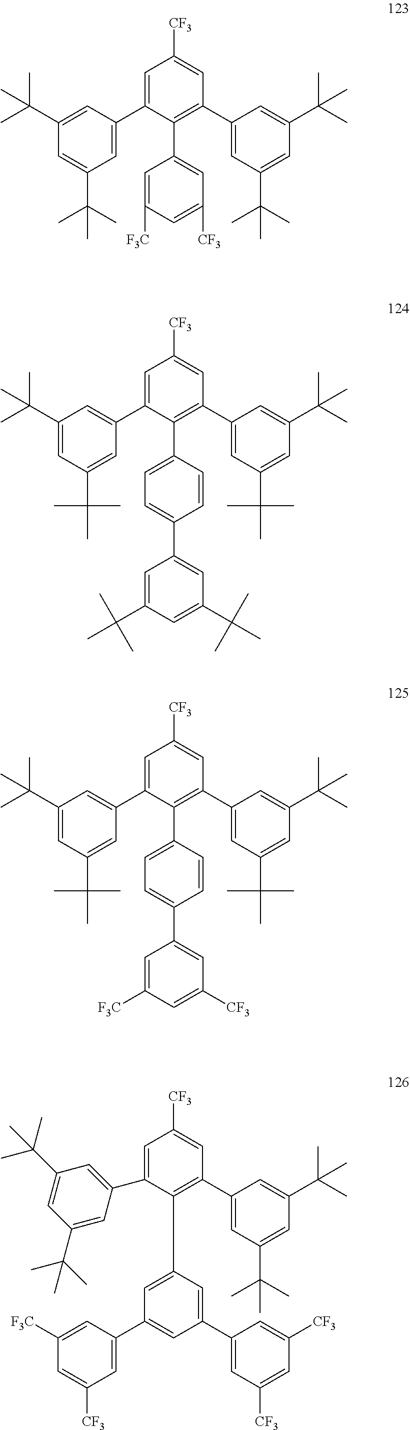

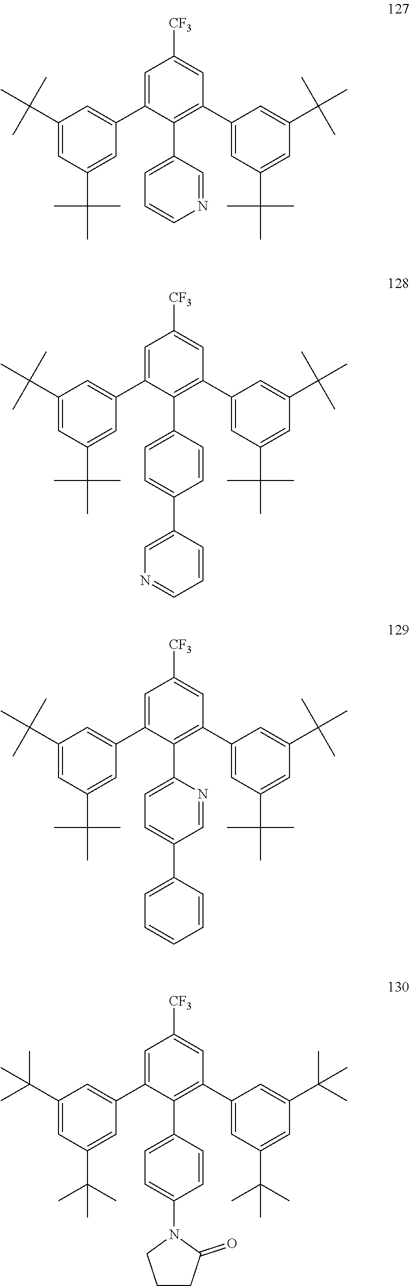

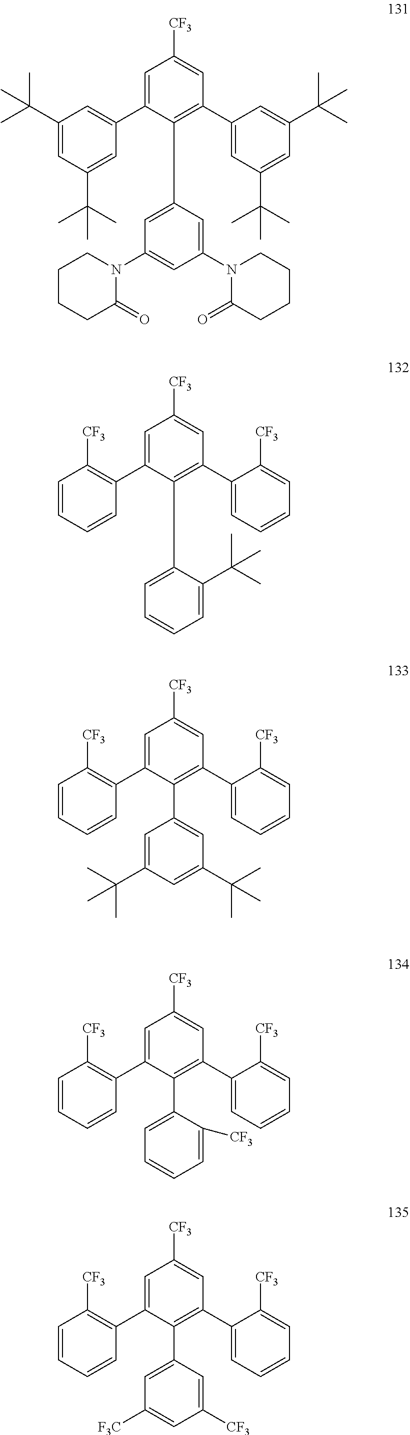 [SMILES: CC(C)(C)c1cc(-c2cc(C(F)(F)F)cc(-c3cc(C(C)(C)C)cc(C(C)(C)C)c3)c2-c2cc(N3CCCCC3=O)cc(N3CCCCC3=O)c2)cc(C(C)(C)C)c1 ; CC(C)(C)c1ccccc1-c1c(-c2ccccc2C(F)(F)F)cc(C(F)(F)F)cc1-c1ccccc1C(F)(F)F ; CC(C)(C)c1cc(-c2c(-c3ccccc3C(F)(F)F)cc(C(F)(F)F)cc2-c2ccccc2C(F)(F)F)cc(C(C)(C)C)c1 ; FC(F)(F)c1cc(-c2ccccc2C(F)(F)F)c(-c2ccccc2C(F)(F)F)c(-c2ccccc2C(F)(F)F)c1 ; FC(F)(F)c1cc(-c2c(-c3ccccc3C(F)(F)F)cc(C(F)(F)F)cc2-c2ccccc2C(F)(F)F)cc(C(F)(F)F)c1]

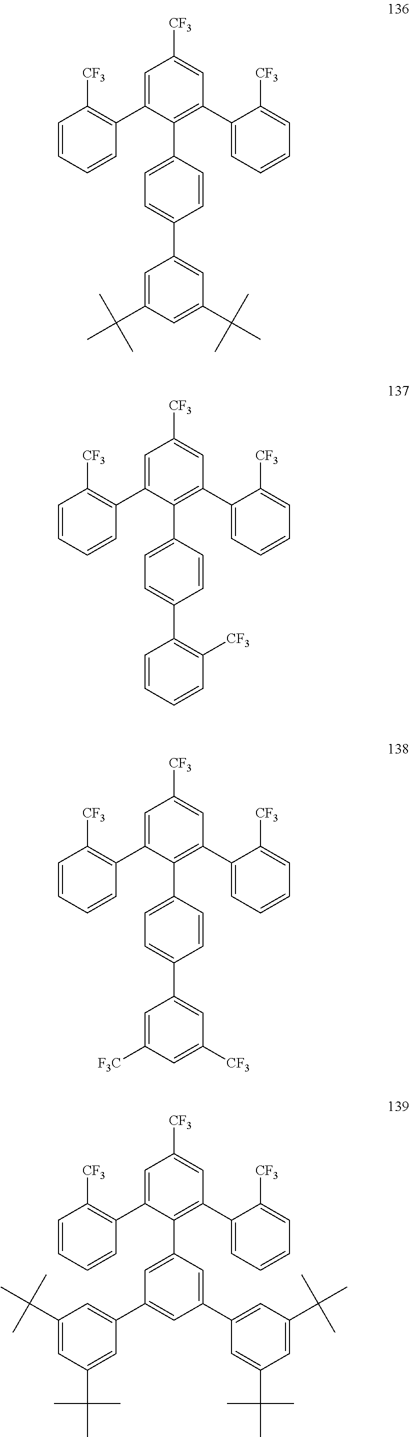 [SMILES: CC(C)(C)c1cc(-c2ccc(-c3c(-c4ccccc4C(F)(F)F)cc(C(F)(F)F)cc3-c3ccccc3C(F)(F)F)cc2)cc(C(C)(C)C)c1 ; FC(F)(F)c1cc(-c2ccccc2C(F)(F)F)c(-c2ccc(-c3ccccc3C(F)(F)F)cc2)c(-c2ccccc2C(F)(F)F)c1 ; FC(F)(F)c1cc(-c2ccc(-c3c(-c4ccccc4C(F)(F)F)cc(C(F)(F)F)cc3-c3ccccc3C(F)(F)F)cc2)cc(C(F)(F)F)c1 ; CC(C)(C)c1cc(-c2cc(-c3cc(C(C)(C)C)cc(C(C)(C)C)c3)cc(-c3c(-c4ccccc4C(F)(F)F)cc(C(F)(F)F)cc3-c3ccccc3C(F)(F)F)c2)cc(C(C)(C)C)c1]

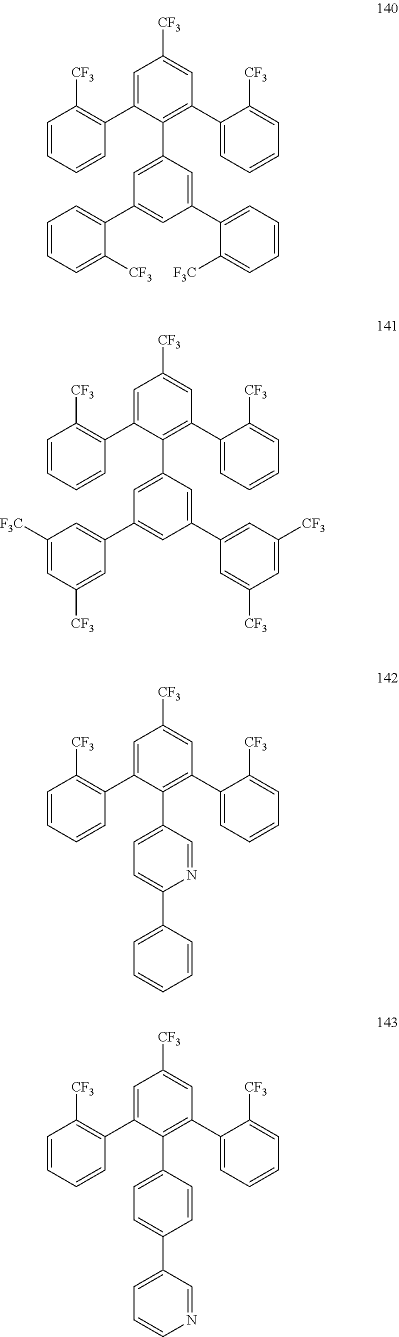

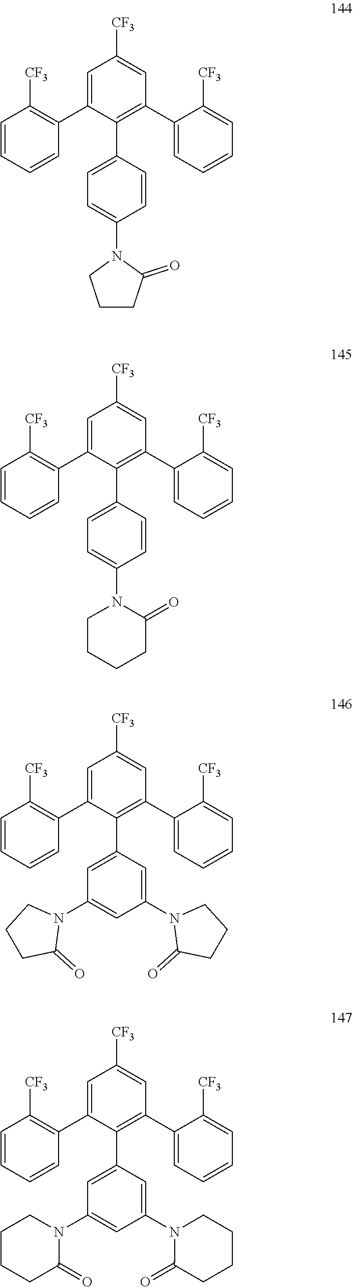 [SMILES: O=C1CCCN1c1ccc(-c2c(-c3ccccc3C(F)(F)F)cc(C(F)(F)F)cc2-c2ccccc2C(F)(F)F)cc1 ; O=C1CCCCN1c1ccc(-c2c(-c3ccccc3C(F)(F)F)cc(C(F)(F)F)cc2-c2ccccc2C(F)(F)F)cc1 ; O=C1CCCN1c1cc(-c2c(-c3ccccc3C(F)(F)F)cc(C(F)(F)F)cc2-c2ccccc2C(F)(F)F)cc(N2CCCC2=O)c1 ; O=C1CCCCN1c1cc(-c2c(-c3ccccc3C(F)(F)F)cc(C(F)(F)F)cc2-c2ccccc2C(F)(F)F)cc(N2CCCCC2=O)c1]

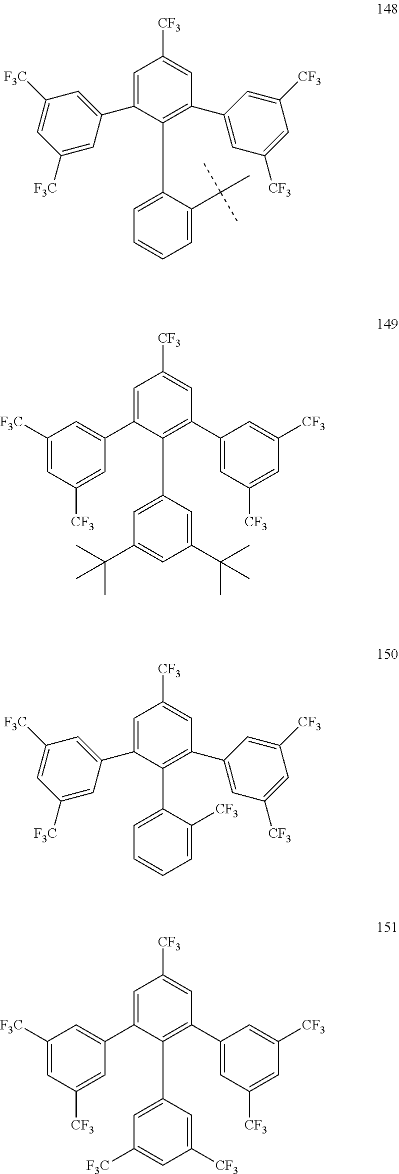

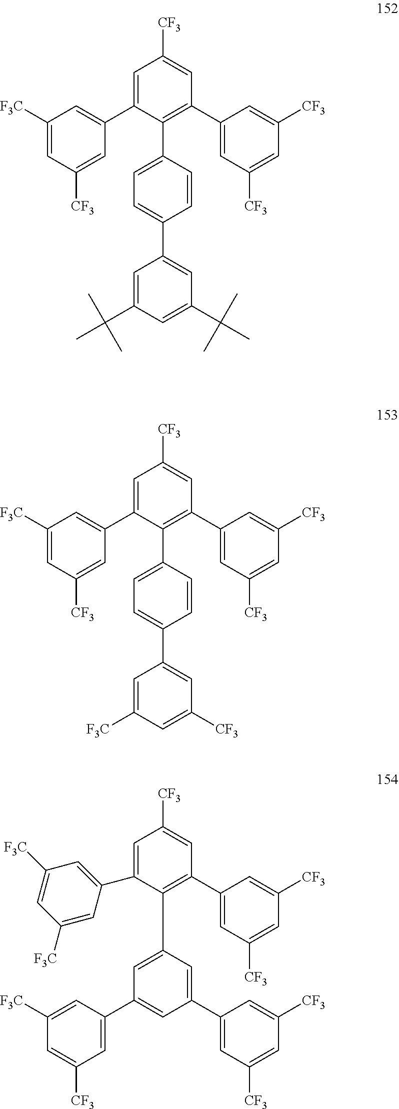 [SMILES: CC(C)(C)c1cc(-c2ccc(-c3c(-c4cc(C(F)(F)F)cc(C(F)(F)F)c4)cc(C(F)(F)F)cc3-c3cc(C(F)(F)F)cc(C(F)(F)F)c3)cc2)cc(C(C)(C)C)c1 ; FC(F)(F)c1cc(-c2ccc(-c3c(-c4cc(C(F)(F)F)cc(C(F)(F)F)c4)cc(C(F)(F)F)cc3-c3cc(C(F)(F)F)cc(C(F)(F)F)c3)cc2)cc(C(F)(F)F)c1 ; FC(F)(F)c1cc(-c2cc(-c3cc(C(F)(F)F)cc(C(F)(F)F)c3)cc(-c3c(-c4cc(C(F)(F)F)cc(C(F)(F)F)c4)cc(C(F)(F)F)cc3-c3cc(C(F)(F)F)cc(C(F)(F)F)c3)c2)cc(C(F)(F)F)c1]

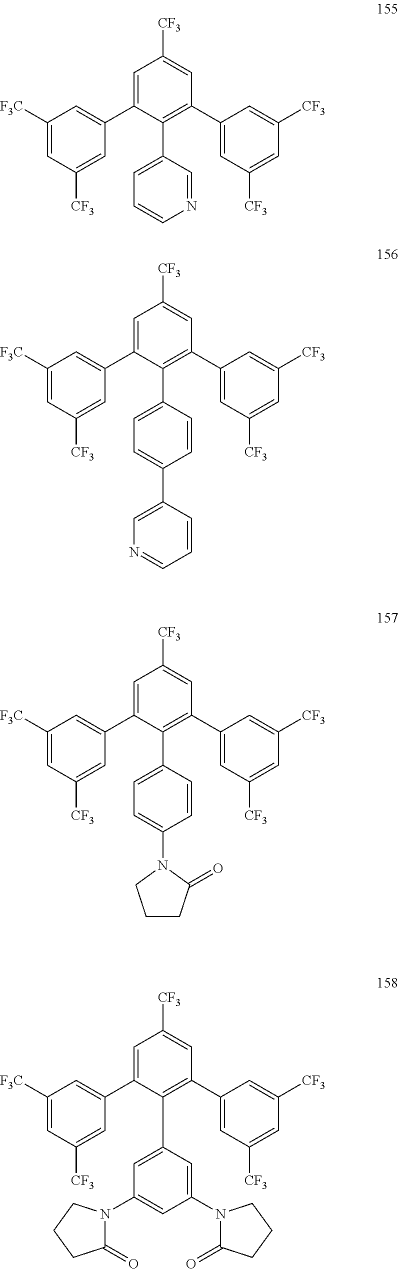

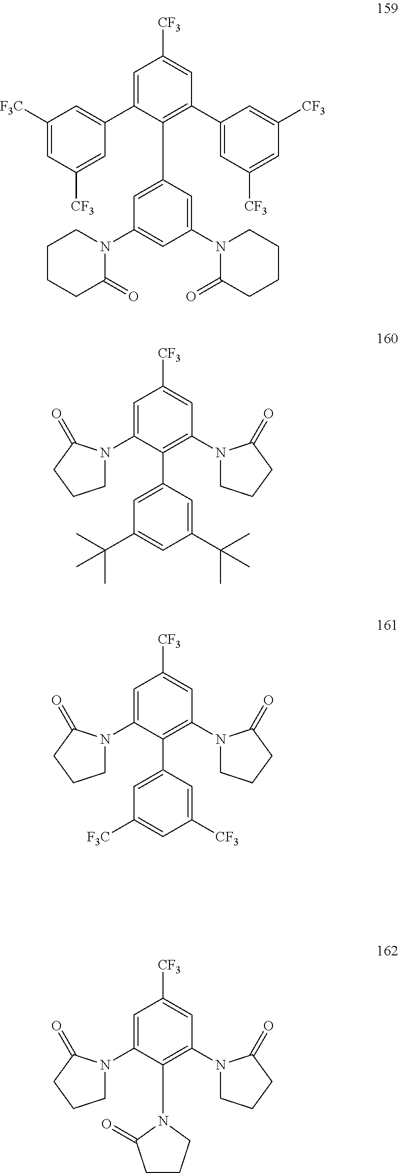 [SMILES: O=C1CCCCN1c1cc(-c2c(-c3cc(C(F)(F)F)cc(C(F)(F)F)c3)cc(C(F)(F)F)cc2-c2cc(C(F)(F)F)cc(C(F)(F)F)c2)cc(N2CCCCC2=O)c1 ; CC(C)(C)c1cc(-c2c(N3CCCC3=O)cc(C(F)(F)F)cc2N2CCCC2=O)cc(C(C)(C)C)c1 ; O=C1CCCN1c1cc(C(F)(F)F)cc(N2CCCC2=O)c1-c1cc(C(F)(F)F)cc(C(F)(F)F)c1 ; O=C1CCCN1c1cc(C(F)(F)F)cc(N2CCCC2=O)c1N1CCCC1=O]

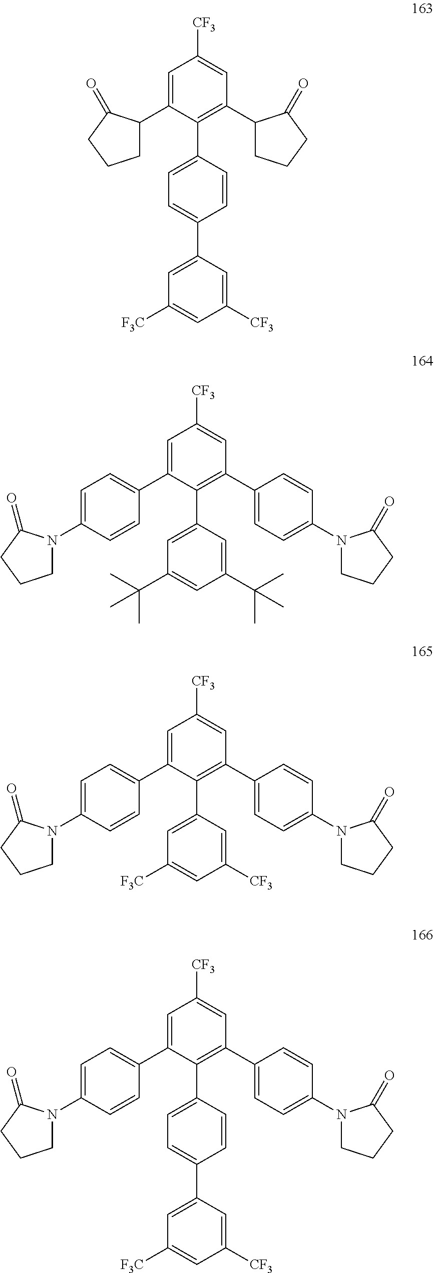 [SMILES: O=C1CCCC1c1cc(C(F)(F)F)cc(C2CCCC2=O)c1-c1ccc(-c2cc(C(F)(F)F)cc(C(F)(F)F)c2)cc1 ; CC(C)(C)c1cc(-c2c(-c3ccc(N4CCCC4=O)cc3)cc(C(F)(F)F)cc2-c2ccc(N3CCCC3=O)cc2)cc(C(C)(C)C)c1 ; O=C1CCCN1c1ccc(-c2cc(C(F)(F)F)cc(-c3ccc(N4CCCC4=O)cc3)c2-c2cc(C(F)(F)F)cc(C(F)(F)F)c2)cc1 ; O=C1CCCN1c1ccc(-c2cc(C(F)(F)F)cc(-c3ccc(N4CCCC4=O)cc3)c2-c2ccc(-c3cc(C(F)(F)F)cc(C(F)(F)F)c3)cc2)cc1]

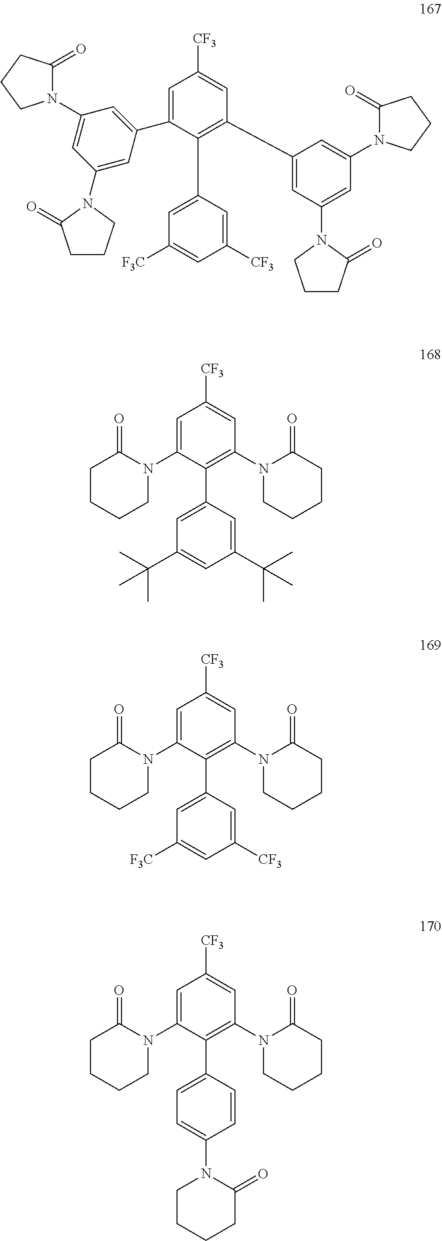 [SMILES: O=C1CCCN1c1cc(-c2cc(C(F)(F)F)cc(-c3cc(N4CCCC4=O)cc(N4CCCC4=O)c3)c2-c2cc(C(F)(F)F)cc(C(F)(F)F)c2)cc(N2CCCC2=O)c1 ; CC(C)(C)c1cc(-c2c(N3CCCCC3=O)cc(C(F)(F)F)cc2N2CCCCC2=O)cc(C(C)(C)C)c1 ; O=C1CCCCN1c1cc(C(F)(F)F)cc(N2CCCCC2=O)c1-c1cc(C(F)(F)F)cc(C(F)(F)F)c1 ; O=C1CCCCN1c1ccc(-c2c(N3CCCCC3=O)cc(C(F)(F)F)cc2N2CCCCC2=O)cc1]

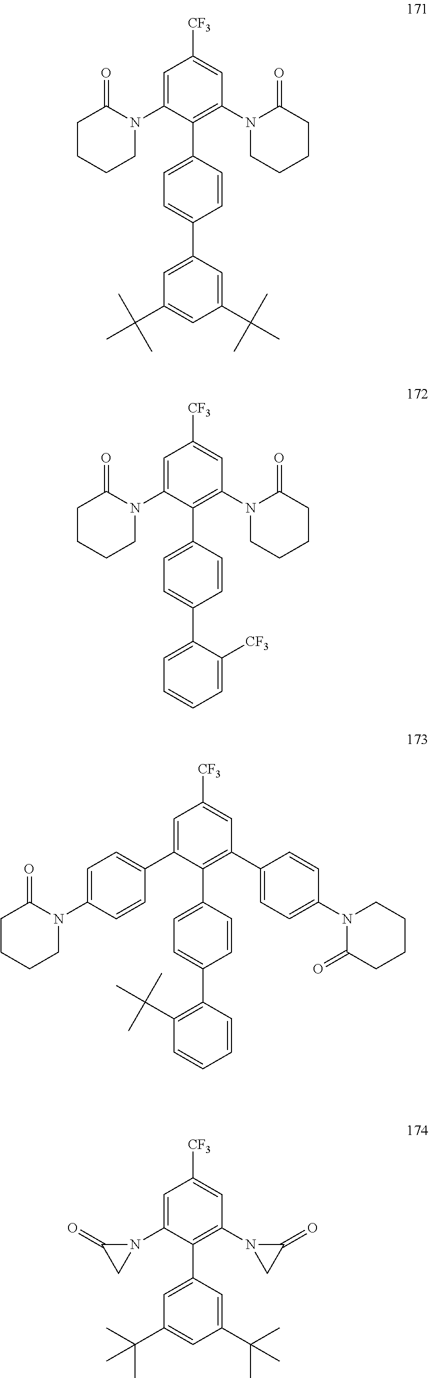 [SMILES: CC(C)(C)c1cc(-c2ccc(-c3c(N4CCCCC4=O)cc(C(F)(F)F)cc3N3CCCCC3=O)cc2)cc(C(C)(C)C)c1 ; O=C1CCCCN1c1cc(C(F)(F)F)cc(N2CCCCC2=O)c1-c1ccc(-c2ccccc2C(F)(F)F)cc1 ; CC(C)(C)c1ccccc1-c1ccc(-c2c(-c3ccc(N4CCCCC4=O)cc3)cc(C(F)(F)F)cc2-c2ccc(N3CCCCC3=O)cc2)cc1 ; CC(C)(C)c1cc(-c2c(N3CC3=O)cc(C(F)(F)F)cc2N2CC2=O)cc(C(C)(C)C)c1]

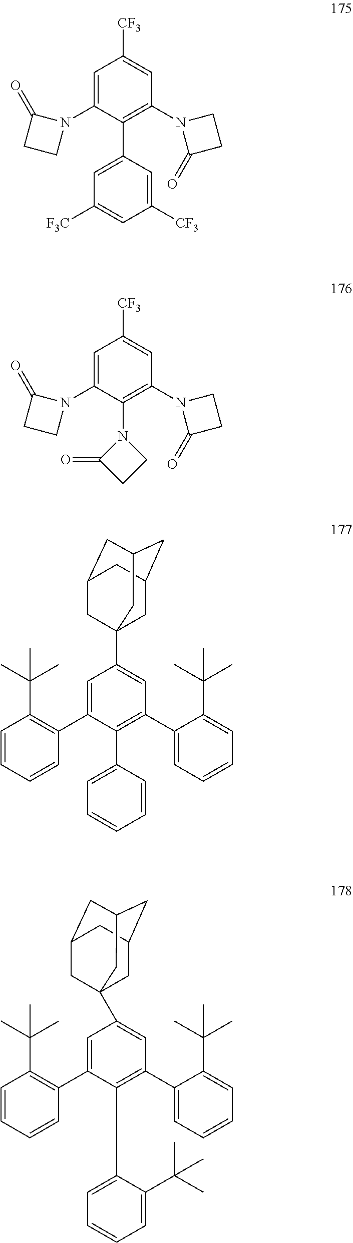

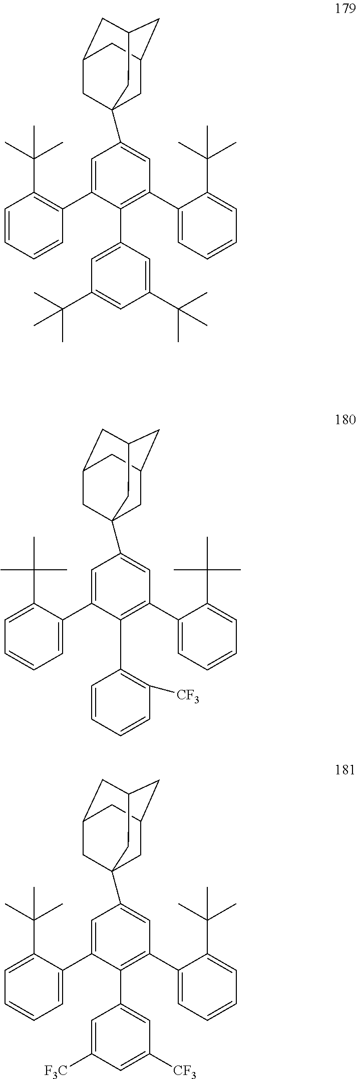

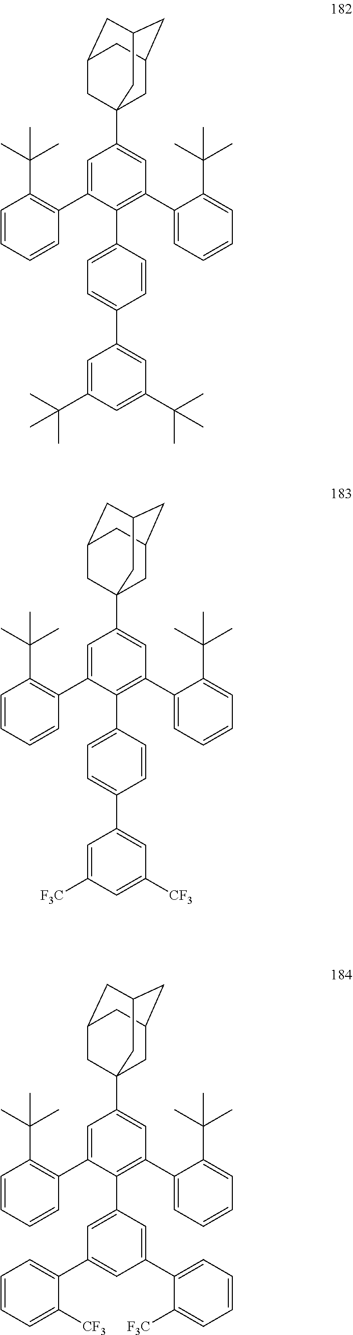

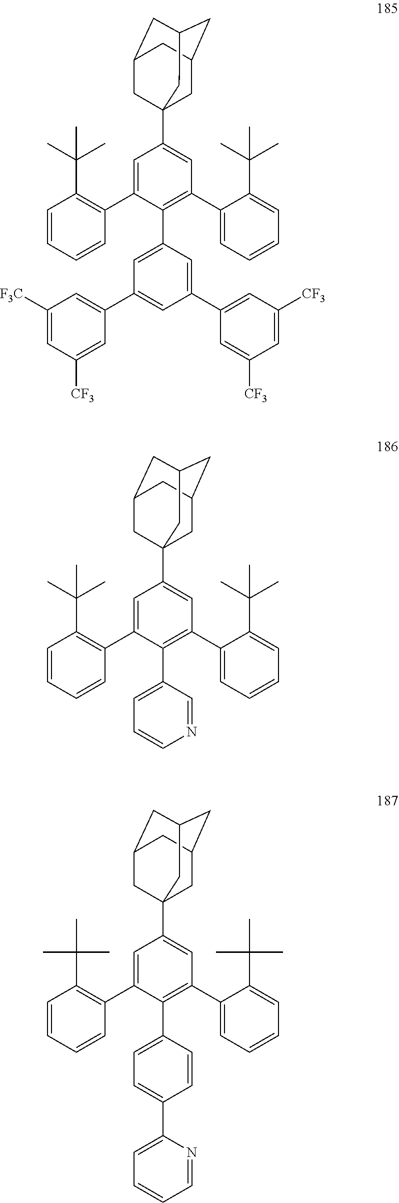

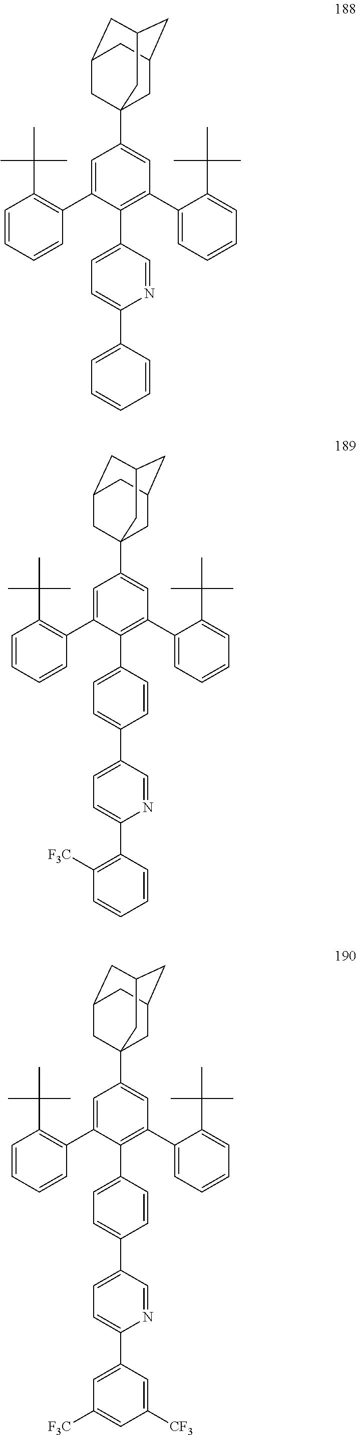

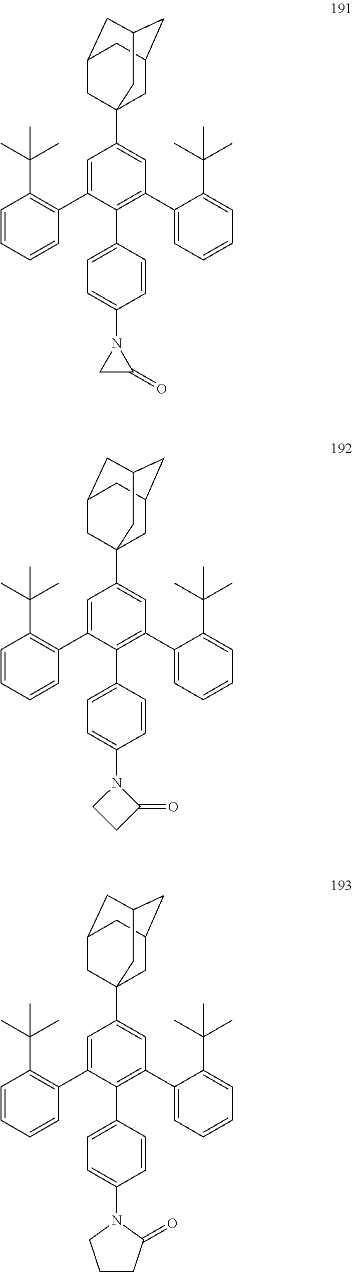 [SMILES: CC(C)(C)c1ccccc1-c1cc(C23CC4CC(CC(C4)C2)C3)cc(-c2ccccc2C(C)(C)C)c1-c1ccc(N2CC2=O)cc1 ; CC(C)(C)c1ccccc1-c1cc(C23CC4CC(CC(C4)C2)C3)cc(-c2ccccc2C(C)(C)C)c1-c1ccc(N2CCC2=O)cc1 ; CC(C)(C)c1ccccc1-c1cc(C23CC4CC(CC(C4)C2)C3)cc(-c2ccccc2C(C)(C)C)c1-c1ccc(N2CCCC2=O)cc1]

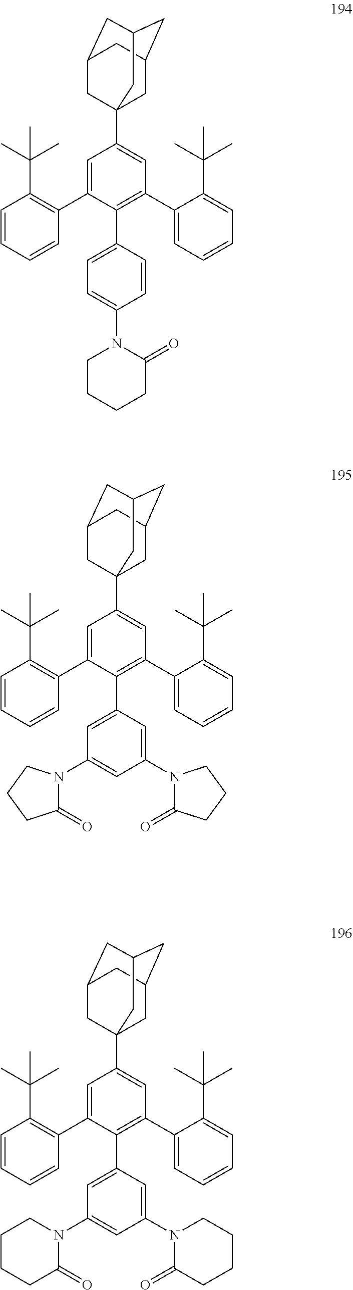 [SMILES: CC(C)(C)c1ccccc1-c1cc(C23CC4CC(CC(C4)C2)C3)cc(-c2ccccc2C(C)(C)C)c1-c1ccc(N2CCCCC2=O)cc1 ; CC(C)(C)c1ccccc1-c1cc(C23CC4CC(CC(C4)C2)C3)cc(-c2ccccc2C(C)(C)C)c1-c1cc(N2CCCC2=O)cc(N2CCCC2=O)c1 ; CC(C)(C)c1ccccc1-c1cc(C23CC4CC(CC(C4)C2)C3)cc(-c2ccccc2C(C)(C)C)c1-c1cc(N2CCCCC2=O)cc(N2CCCCC2=O)c1]

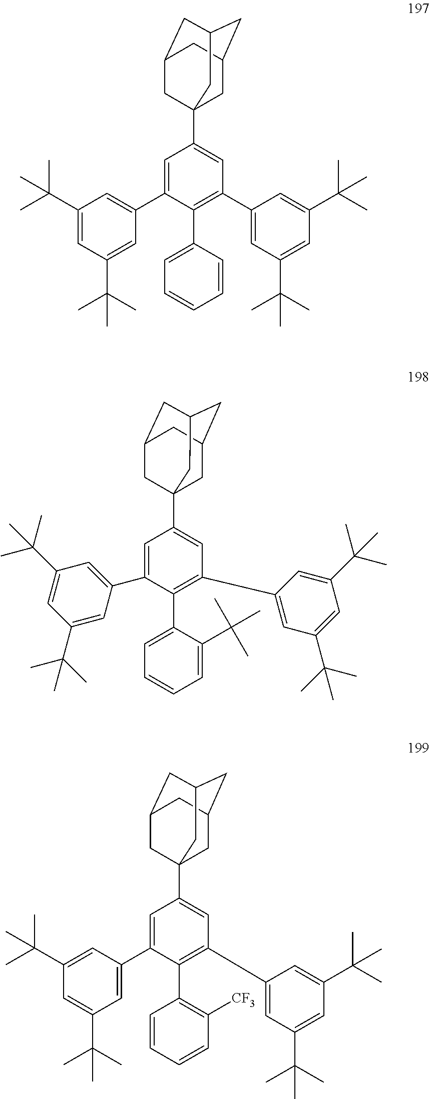

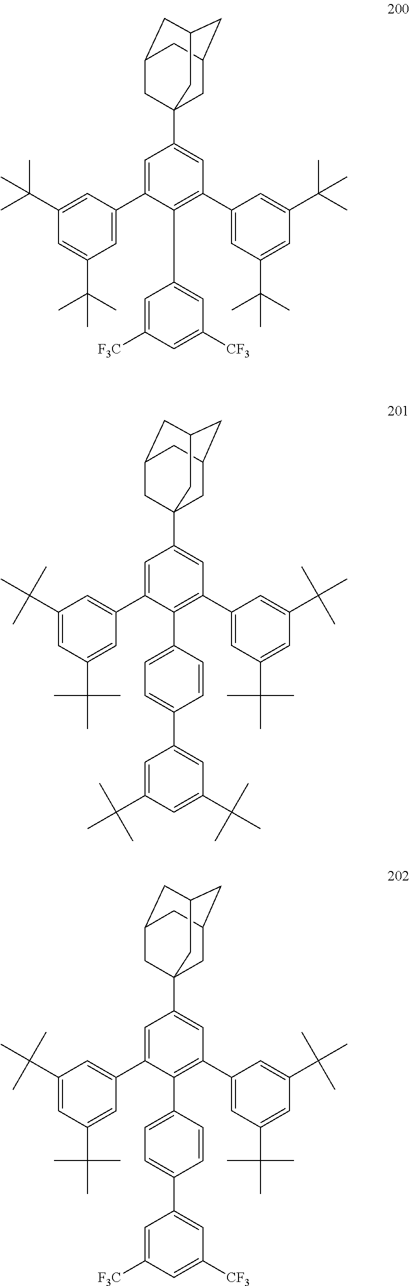

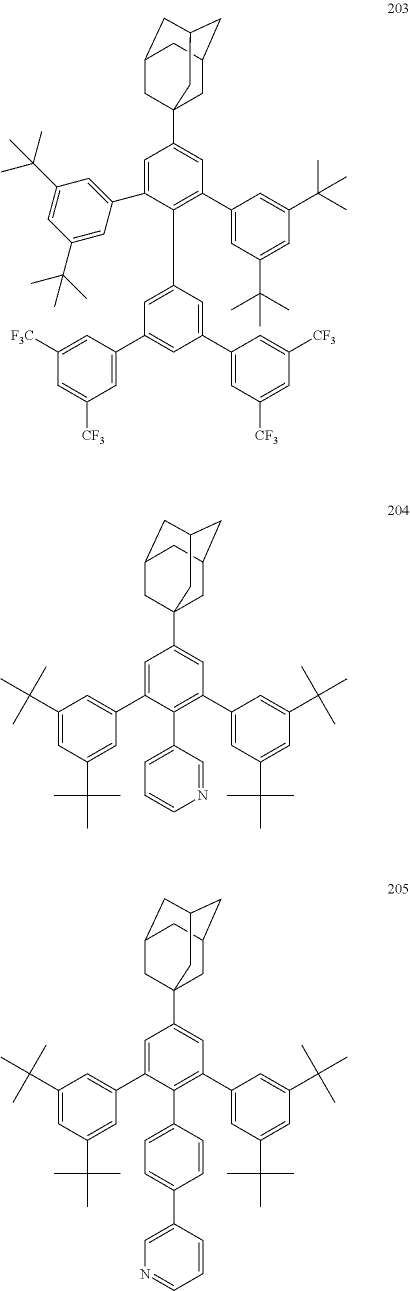

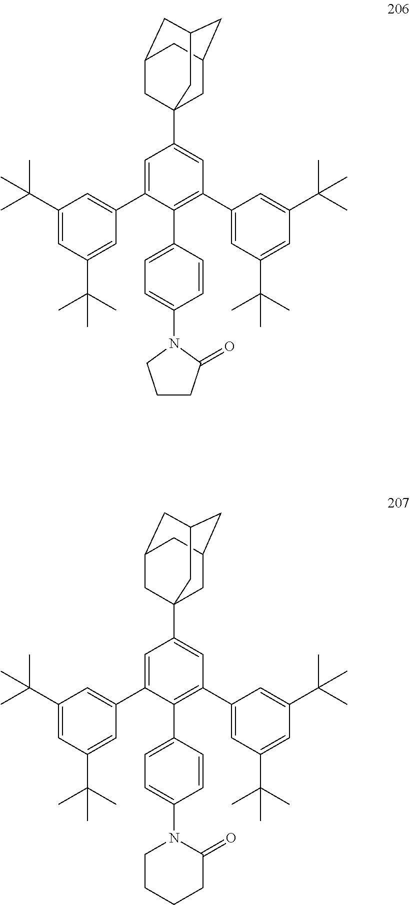

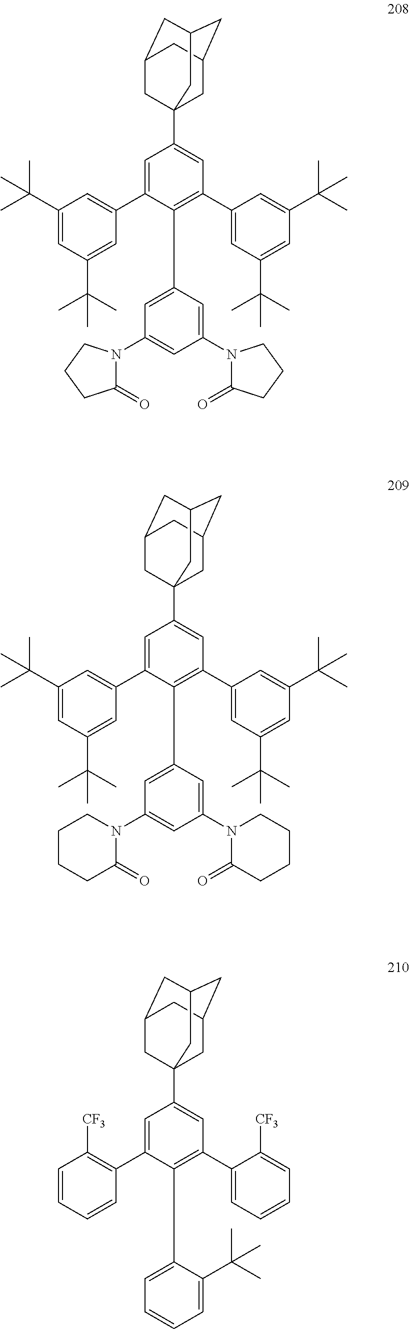

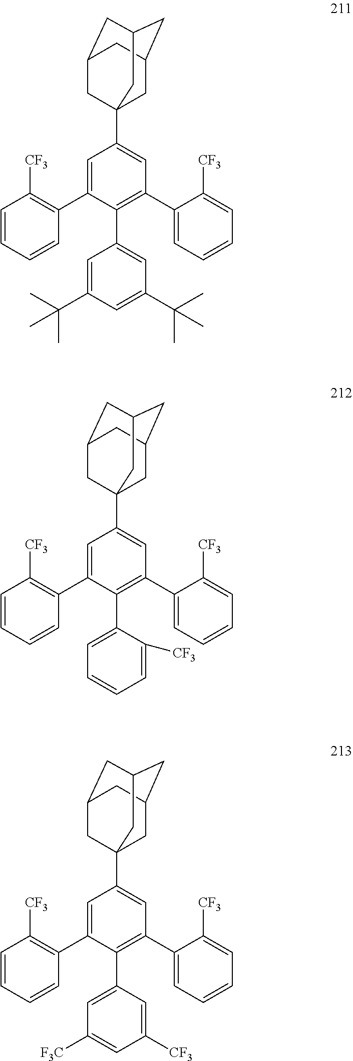 [SMILES: CC(C)(C)c1cc(-c2c(-c3ccccc3C(F)(F)F)cc(C34CC5CC(CC(C5)C3)C4)cc2-c2ccccc2C(F)(F)F)cc(C(C)(C)C)c1 ; FC(F)(F)c1ccccc1-c1cc(C23CC4CC(CC(C4)C2)C3)cc(-c2ccccc2C(F)(F)F)c1-c1ccccc1C(F)(F)F ; FC(F)(F)c1cc(-c2c(-c3ccccc3C(F)(F)F)cc(C34CC5CC(CC(C5)C3)C4)cc2-c2ccccc2C(F)(F)F)cc(C(F)(F)F)c1]

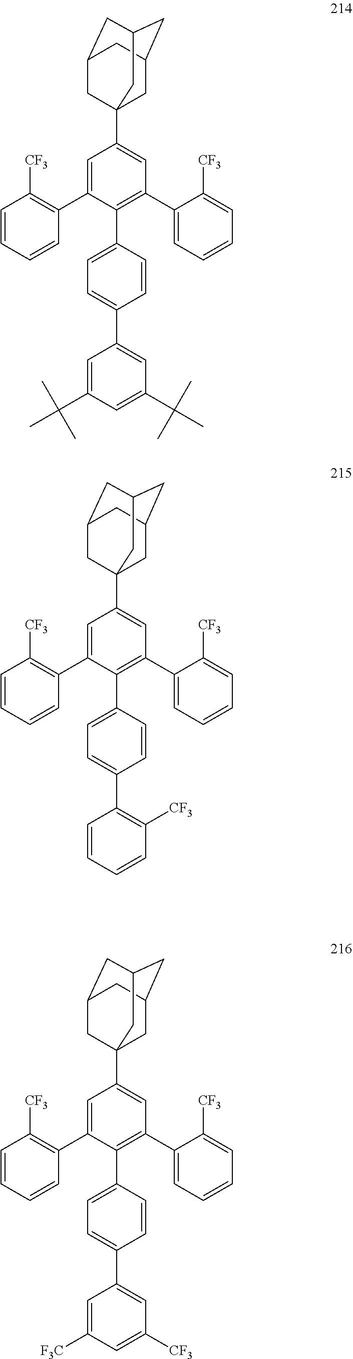

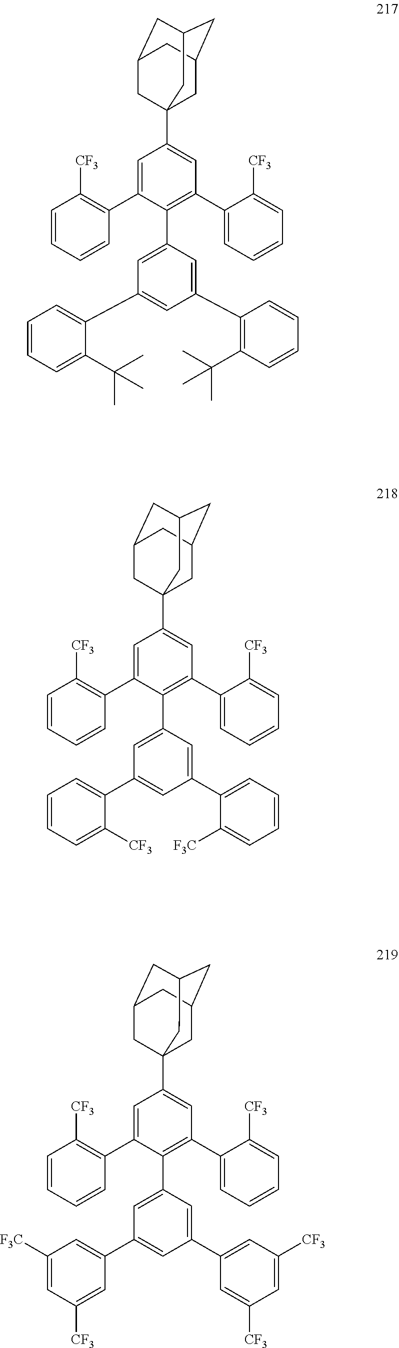 [SMILES: CC(C)(C)c1ccccc1-c1cc(-c2ccccc2C(C)(C)C)cc(-c2c(-c3ccccc3C(F)(F)F)cc(C34CC5CC(CC(C5)C3)C4)cc2-c2ccccc2C(F)(F)F)c1 ; FC(F)(F)c1ccccc1-c1cc(-c2ccccc2C(F)(F)F)cc(-c2c(-c3ccccc3C(F)(F)F)cc(C34CC5CC(CC(C5)C3)C4)cc2-c2ccccc2C(F)(F)F)c1 ; FC(F)(F)c1cc(-c2cc(-c3cc(C(F)(F)F)cc(C(F)(F)F)c3)cc(-c3c(-c4ccccc4C(F)(F)F)cc(C45CC6CC(CC(C6)C4)C5)cc3-c3ccccc3C(F)(F)F)c2)cc(C(F)(F)F)c1]

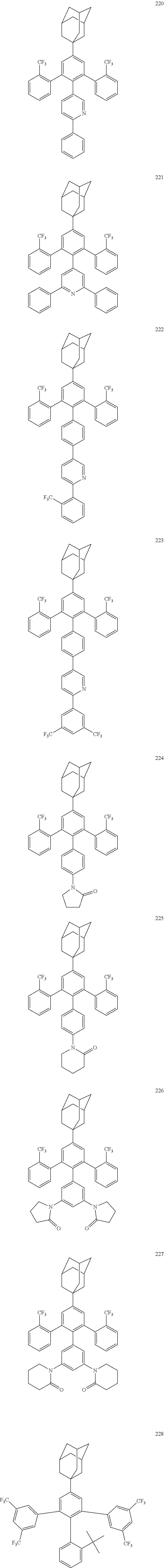

[chemical structure]

[chemical structure]

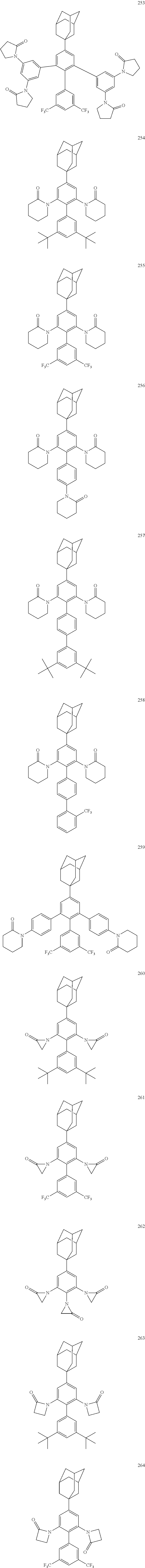

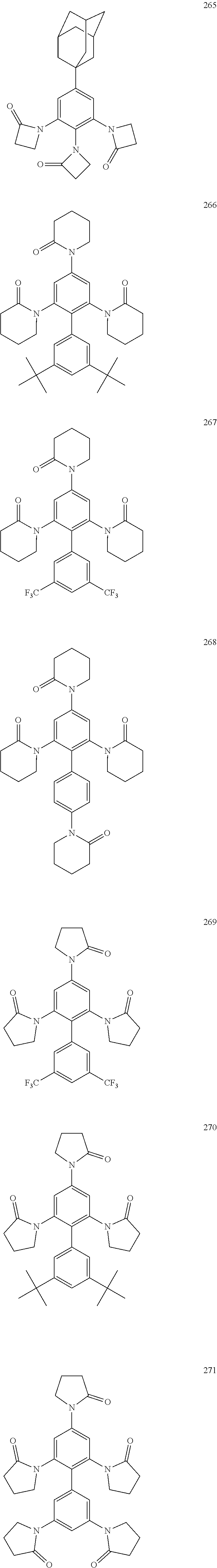

4. An organic light-emitting device comprising a first electrode, a second electrode, and an organic material layer having one or more layers disposed between the first electrode and the second electrode,
further comprising a light efficiency improving layer (capping layer) formed on at least one side opposite to the organic layer at the top or bottom of the first electrode and the second electrode,
wherein the light efficiency improving layer comprises the compound represented by [Formula I] above.

5. The organic light-emitting device of claim 4, wherein the light efficiency improving layer is formed on at least one of the bottom of the first electrode and the top of the second electrode.

6. The organic light-emitting device of claim 4, wherein the light efficiency improving layer is composed of a plurality of layers having different refractive indices, and any one of the plurality of light efficiency improving layers comprises the compound represented by [Formula I] above.
